(19) United States
(12) Patent Application Publication
(10) Pub. No.: US 2021/0054018 A1
(43) Pub. Date: Feb. 25, 2021

(54) TRANSFER RNA LIGAND ADDUCT LIBRARIES

(71) Applicant: GALEN BIOTECHNOLOGIES, LLC, APTOS, CA (US)

(72) Inventors: Christopher J. MURRAY, Aptos, CA (US); Bradley SPATOLA, Cupertino, CA (US)

(21) Appl. No.: 16/070,503

(22) PCT Filed: Jan. 13, 2017
(86) PCT No.: PCT/US2017/013561

§ 371 Date: Jul. 16, 2018

(51) Int. Cl. C07H 21/04; C07K 1/04; C12N 9/00; C40B 50/06

(52) U.S. Cl. CPC C07H 21/04; C40B 50/06; C12N 9/93; C07K 1/047

(57) ABSTRACT

The present invention is drawn to, among other things, compositions of matter and methods for producing an aminoacyl-tRNA analogue comprising an adaptor tRNA and modified amino acid for ribosome-directed translation in vitro.

### CROSS REFERENCE TO RELATED APPLICATIONS

This application is related to and claims the priority of U.S. Provisional Patent Application No. 62/279,273, filed Jan. 15, 2016, which is hereby incorporated herein by reference in its entirety.

### REFERENCE TO SEQUENCE LISTING SUBMITTED VIA EFS-WEB

This application is being filed electronically via EFS-Web and includes an electronically submitted sequence listing in .txt format. The .txt file contains a sequence listing entitled “GAL_002_SeqListing.txt” created on Jan. 15, 2016 and is 1 kilobyte in size. The sequence listing contained in this .txt file is part of the specification and is hereby incorporated by reference herein in its entirety.

### FIELD OF THE INVENTION

The invention relates to methods of optimizing the properties of aminoacyl transfer RNA molecules, optimized aminoacyl transfer RNA molecules, methods for using optimized aminoacyl transfer RNA molecules, and compositions which include aminoacyl transfer RNA molecules.

### BACKGROUND OF THE INVENTION

Biological systems are unparalleled in their ability to synthesize polypeptides of enormous sequence diversity from 20 natural amino acid building blocks. A polypeptide of 100 amino acids has 20¹⁰⁰ possible combinations of mutations. Theory predicts 10⁶⁰ possible small molecules in chemical space. These numbers are too large to explore by conventional drug discovery approaches. It would be advantageous to reengineer ribosome-directed translation to encode vast numbers of diverse chemical structures which would allow the generation, selection, and screening of combinatorial biopolymer libraries displaying side chains with non-canonical function (Brustad, E. M. and Arnold, F. H., Curr Opin Chem Biol (2011) 15:201-210; Frankel, A., et al., Curr Opin Struct Biol (2003) 13:506-512). Ribosome-directed translation machinery is highly complex, making the incorporation of new chemical moieties into polypeptides difficult. For example, translated polypeptide side chain structures must pass through an ˜80 Å long ribosome exit tunnel that limits the size, shape, and charge of chemical side chain moieties that can be incorporated into a polymer backbone (Voss, N. R., et al., J Mol Biol (2006) 360:893-906; Hohsaka, T., et al., FEBS Lett (1993) 335:47-50; Lu, J. and Deutsch, C., J Mol Biol (2008) 384:73-86).

Amino acids and non-canonical or non-natural amino acids (nnAAs; e.g., modified amino acids) can be used for protein synthesis (Noren, C., et al., Science (1989) 244:182-188; Chapeville, F., et al., Proc Natl Acad Sci USA (1962) 48:1086-1092). It would be useful to have nnAA function as a substrate for an aminoacyl-tRNA synthetase enzyme (aaRS). However, aaRSs' are very precise enzymes which acylate only a specific tRNA with their cognate amino acid, and do not recognize non-cognate amino acids or tRNA. Thus misacylation of tRNA with extremely diverse non-natural amino acids is very difficult to achieve. Methods also have difficulty selectively incorporating different non-natural amino acids because the number of non-cognate aaRS/tRNA/nnAA interactions is limited to a finite number of tRNA identity elements as well as aaRS and nnAA interactions and should be expected to produce a concomitant decrease in the fidelity of the genetic code as the number of nnAAs increases (Ardell, D. H., FEBS Lett (2010) 584:325-333; Yarus, M., Nat New Biol (1972) 239:106-108; Schlippe, Y. V., et al., J Am Chem Soc (2012) 134:10469-10477)

Chemically misacylated tRNAs have been prepared by various methods (Suga, H., et al., J Am Chem Soc (1998) 120:1151-1156; Hecht, S. M., Protein Engineering (2009) 22:255-270 U.S. Pat. No. 7,288,372), and used to introduce non-natural amino acids into polypeptides. In some cases side chains of canonical aminoacyl-tRNAs are enzymatically (Ibba, M., et al., Trends in biochemical sciences (2000) 25:311-316) or chemically (Kurzchalia, T. V., et al., EP0234799 (1987); Fahnestock, S. and Rich, A., Science (1971) 173:340-343) modified prior to translation. For example, Alder, N. N., et al., Cell (2008) 134:439-450 describe methods for generating Cys-tRNA^{Cys} functionalized with N,N′-dimethyl-N-(iodoacetyl)-N′-(7-nitrobenz-2-oxa-1,3-diazol-4-yl) ethylenediamine (NBS) via a iodoacetyl linker. The NBS-Cys-tRNA^{Cys} is compatible with translation via the ribosome exit tunnel (e.g., van der Waals volume ˜342 Å³). Treco, D. A. and Ricardo, A. in WO 2013/019794, incorporated here as reference, teach that chemical modification of non-canonical aminoacyl-tRNAs may be used to stop translation by chemically linking the mRNA to the encoded polypeptide during ribosome-directed translation. In spite of this, it is thought that ribosome-directed translation approaches are limited to a few simple derivatives of the common twenty amino acids (Franzini, R. M., et al., Accounts Chem. Res. (2014) 47:1247-1255).

The above referenced methods have a number of deficiencies, many of which are solved by the present invention. The acylated-tRNAs are prepared in low yield, from multiple complex chemical and biochemical steps, and require extensive chromatographic purification at each step. Furthermore, methods which require canonical amino acid side chain chemical reactivity can be incompatible with tRNA or protein stability, or translation, or limit the process to one-site per gene product, and must compete with canonical aminoacyl-tRNA^{AA} during translation, thus limiting the fidelity of the final translated product (Seebeck, F. P. and Szostak, J. W., J Am Chem Soc (2006) 128:7150-7151). Attempts to address fidelity using nonsense codon suppression or reconstituted translation systems lacking specific AA-tRNA^{AA} have been hampered by low yields and low fidelity of polymers produced by ribosome-directed translation (Schlippe, Y. V., et al., J Am Chem Soc (2012) 134:10469-10477; Wang, H. H., et al., ACS Synth Biol (2012) 1:43-52; Shimizu, Y., et al., Nat Biotechnol (2001) 19:751-755; Antonczak, A. K., et al., Proc Natl Acad Sci USA (2011) 108:1320-1325). Wrenn, S. J. and Harbury, P. B., Ann Rev Biochem (2007) 76:331-349 teach that in vitro translation in a cell-free extract using a non-canonical aminoacyl-tRNA_{CUA} have little utility for selection of non-canonical peptide libraries.

It would be advantageous to have large libraries. Large libraries provide an advantage for directed evolution applications, in that chemical space can be explored to a greater depth around any given starting chemical structure and sequence. In this context, the use of aminoacyl-tRNAs as stoichiometric reagents may be considered to limit the amount of polypeptide that can be produced in vitro. Concentrations of misacylated-tRNA (up to 2 mg/mL) used in in vitro protein synthesis reactions would require a considerable amount of tRNA for large libraries >10″ (20-200 mg), limiting library complexity by the amount of misacylated-tRNAs produced; see Merryman, C. F and Green, R. D, United States Patent Application 2006/0252051 and WO 03046195, incorporated by reference herein.

Other problems with previous methods include instability of linkers, post-translational labeling of ribosome-displayed libraries produced in transcription-translation lysates require complex and unique analytical QC of each library scaffold produced (see Li, S. and Roberts, R. W., Chem Biol (2003) 10:233-239), and short transcription/translation times are incompatible with complete labeling of translated polypeptides, again leading to loss of fidelity due to mixtures of fully-reacted and unreacted non-natural amino acids for a single sequence. Furthermore, current systems for producing aminoacyl-tRNAs are limited in their ability to generate both sufficient quantities of misacylated tRNAs and chemically complex libraries of misacylated tRNAs for efficient encoded translation.

It would be useful to assemble non-natural chemical structures for libraries using as few synthetic steps as possible, in as high a yield as possible, and in a chemically scalable manner. There is a significant need for compositions and methods that would allow one to expedite ribosome-directed synthesis and screening of large encoded combinatorial libraries where large numbers of reactions, e.g., 50-10,000 or more, are carried out at each reaction step, e.g., for building chemically diverse libraries that involve a small number of successive synthesis steps. In order to achieve high library diversity each reaction step must encompass a large number of efficient chemical reactions, e.g., 100-1,000 or more different reactions at each reaction step, thus achieving, for example, 1×10⁶ (three reaction steps, 100 different reactions/step) or 1×10⁹ (three reaction steps, 1,000 different reactions/steps) for total library size. Despite considerable effort over many years by many workers skilled in the art, an efficient solution to the molecular recognition problems posed by drug discovery using translated non-canonical amino acid libraries remains elusive.

### SUMMARY OF THE INVENTION

The present invention is drawn to, among other things, compositions of matter and methods for producing an aminoacyl-tRNA analogue comprising an adaptor tRNA and modified amino acid for ribosome-directed translation in vitro.

In a first aspect, the invention is drawn to an aminoacyl-tRNA analogue capable of ribosome-directed translation and methods for producing aminoacyl-tRNA analogues comprising an adaptor tRNA and having a structure:

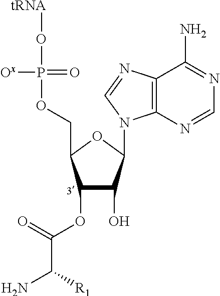

wherein R₁ is a ligand adduct moiety linked to a non-natural amino acid side chain.

In a second aspect, the invention is drawn to an acyl-tRNA analogue capable of ribosome-directed translation having a structure:

tRNA-A-z-L

wherein:
tRNA has a 3′ terminus to which at least one hydroxyacyl or aminoacyl group may be transferred;
A is a aminoacyl or α-hydroxyacyl group selected from the group consisting of canonical amino acids, α-hydroxyl acids and non-canonical amino acids with an orthogonally reactive moiety y;
L is a ligand with a reactive moiety x and
z is a covalent linker formed by a reaction of tRNA-A-y with x-L. In one embodiment, the aminoacyl group comprises at least one non-canonical amino acid with an orthogonally reactive moiety y. In another embodiment, the 3′ terminus is cytosine C75 (or its equivalent) of the acyl-tRNA but is not 2′-deoxycytosine.

In a third aspect, the invention is drawn to a method of reacting a starting aminoacyl-tRNA compound represented by a structural formula:

tRNA-A-y

wherein A is a non-canonical amino acid with an orthogonally reactive moiety y, with a ligand, x-L, containing a reactive moiety x, under conditions suitable for a reaction, the method comprising forming a covalent linker between tRNA-A-y and x-L, the covalent linker forming a product wherein the product is an aminoacyl-tRNA analogue capable of ribosome-directed translation. In a preferred embodiment, the 3′ cytosine C75 (or its equivalent) of the acyl-tRNA is not 2′-deoxycytosine.

In a first embodiment of the third aspect, the conditions suitable for a reaction comprise an acidic pH. In a preferred embodiment, the acidic pH is a pH between approximately 1 and 5. In a preferred embodiment, the pH is approximately 5. In a second embodiment of the third aspect the starting aminoacyl-tRNA is produced substantially pure in vitro by enzymatic aminoacylation with an engineered aaRS, a tRNA or a non-canonical amino acid. In a third embodiment of the third aspect, an aminoacyl-tRNA is produced by transcription of a tRNA-ribozyme encoded DNA template with treatment of polynucleotide kinase. In a fourth embodiment of the third aspect, the starting aminoacyl-tRNA is produced substantially pure by a T4 RNA ligase coupling of tRNA(-CA) with a non-canonical aminoacyl-pdCpA.

In a fifth embodiment of the third aspect, the invention is drawn to an in vitro transcription and translation system that comprises aminoacyl transfer RNA molecules of the invention. In an embodiment, a polypeptide containing a site-specific ligand adduct moiety linked to a non-natural amino acid side chain is synthesized by in vitro translation from an mRNA template containing a selector codon at specific sites. In an embodiment, the mRNA is encoded by a DNA template containing a selector codon at specific sites. In vitro translation systems such as the CYTOMIM™ translation system described in U.S. Pat. No. 7,338,789, herein incorporated by reference, or reconstituted transcription/translation systems may be used. In some embodiments, various excipients may be added, attenuated or removed from the translation system. In some embodiments engineered Ef-Tu variants may be added (Doi, Y., et al., J Am Chem Soc (2007) 129:14458-14462). In some embodiments, release factors may be removed or attenuated (Shimizu, Y., et al., Nat Biotechnol (2001) 19:751-755). In some embodiments various chaperones, including folding chaperones, may be added.

In the various embodiments of the invention, x and y may be independently selected from the group consisting of (a) an azide as either x or y and an alkyne as the other; (b) an alkene as either x or y and a thiol or an amine as the other; (c) a vinyl sulfone as either x or y and a thiol or an amine as the other; (d) an α-halo-carbonyl as either x or y and a thiol or an amine as the other; (e) a disulfide as either x or y and a thiol as the other; (f) a carbonyl as either x or y and an alpha-effect amine as the other; (g) an activated carboxylic acid as either x or y and a alkyl or aryl amine as the other; (h) a 1,4-dicarbonyl as either x or y and a alkyl or aryl amine as the other; (i) an aryl halide as either x or y and an alkyl boronate ester as the other, and (j) an aryl halide as either x or y and an alkyne as the other. In some preferred embodiments x and y may react at low pH. In an embodiment, x and y are masked or protected by reactive functional groups. See Protective Groups in Organic Synthesis (Third Edition) Greene, T. W. and Wuts, P. G. M., (2002).

Other reactive groups known to one skilled in the art are intended to be within the scope of the invention. The assignment of such reactive functionalities between x and y may be determined by one skilled in the art based on considerations such as speed of reaction, absence of side reactions in the reaction mixture, reversibility of reaction, product stability, etc. In general, it is not material which chemically reactive group of a given pair of chemically reactive groups is on the transfer RNA unit and which is on the ligand prior to subsequent reaction to form the aminoacyl-tRNA ligand adduct moieties.

In a fourth aspect, the invention is drawn to a library formed by a reaction comprising reacting

a) an aminoacyl-tRNA having formula tRNAₘ-A-y,

wherein the amino aminoacyl-tRNA has a preselected anti-codon, m, and A comprises a preselected non-canonical amino acid comprising an acceptor moiety with a reactive functionality, y, with

b) a plurality of ligand moieties (x-L₁, x-L₂, . . . x-Lₙ), each ligand comprising a donor reactive functionality, x, and one of a plurality of ligand moieties (L₁, L₂, . . . Lₙ),

the reaction occurring under conditions suitable for the reaction, the conditions sufficient to form a plurality, n, of transfer RNA ligands (tRNA₁-A-z-L₁, tRNA₂-A-z-L₁, . . . tRNAₘ-A-z-Lₙ),

wherein z is a linker formed by reaction of x and y.

In the various embodiments of the fourth aspect, x and y may be independently selected from the group consisting of (a) an azide as either x or y and an alkyne as the other; (b) an alkene as either x or y and a thiol or an amine as the other; (c) a vinyl sulfone as either x or y and a thiol or an amine as the other; (d) an α-halo-carbonyl as either x or y and a thiol or an amine as the other; (e) a disulfide as either x or y and a thiol as the other; (f) a carbonyl as either x or y and an alpha-effect amine as the other; (g) an activated carboxylic acid as either x or y and a alkyl or aryl amine as the other; (h) a 1,4-dicarbonyl as either x or y and a alkyl or aryl amine as the other; (i) an aryl halide as either x or y and an alkyl boronate ester as the other, and (j) an aryl halide as either x or y and an alkyne as the other. In some preferred embodiments x and y may react at low pH. In an embodiment, x and y are masked or protected by reactive functional groups.

In a first embodiment of the fourth aspect, the conditions suitable for a reaction comprise an acidic pH. In an embodiment, the acidic pH is a pH between approximately 1 and 5. In an embodiment, the pH is approximately 5.

In a second embodiment of the fourth aspect, the plurality of ligand moieties are unbiased, functionally-biased, target-biased or focused.

In a third embodiment of the fourth aspect, the library is spatially addressed or pooled.

In a fifth aspect, the invention is drawn to a method of screening for a compound that binds to a target, the method comprising:

a) providing a library comprising a plurality of predefined tRNAs that are aminoacylated with a plurality of predefined non-canonical amino acid ligand adducts as set forth herein, wherein the predefined aminoacylated-tRNA non-canonical amino acid ligand adducts are contained in one of a preselected spatially addressed array of vessels;
b) adding a DNA or mRNA template directing the translation of one or more polypeptides site-specifically modified with the predefined non-canonical amino acid ligand adducts;
c) adding a translation system that synthesizes one or more polypeptides site-specifically modified with the predefined non-canonical amino acid ligand adducts;
d) contacting a target with the polypeptides under conditions that permit binding between the target and the polypeptides; and
e) assessing the presence and/or absence of binding between the target and the polypeptides site-specifically with the pre-defined non-canonical amino acid ligand adducts.

In a first embodiment of the fifth aspect, the method comprises an additional step, wherein the additional step is carried out between step (c) and step (d) and the additional step comprises linking the polypeptides to their encoding mRNA sequences. In an embodiment, the translated polypeptides linked to their encoding mRNA are pooled. In the additional step, linking can be used directly or indirectly to assess or quantify the presence and/or absence of binding between a target and the polypeptides with non-canonical amino acid ligand adducts incorporated site-specifically. Linking can be a chemical link (cf. U.S. Pat. No. 6,214,553, WO 2013/019794, and references cited therein), a physical link and/or a physical link that is only temporary cf. Mattheakis, L. C., et al., Proc Natl Acad Sci USA (1994) 91:9022-9026 As an example, a predetermined barcode to the DNA or mRNA template, wherein the predetermined barcode is uniquely associated with a non-canonical amino acid, may be used to identify polypeptides products of the translation system (cf. Tjhung, K. F. et al. Journal of the American Chemical Society (2016) 138, 32-35).

In a second embodiment of the fifth aspect, binding is catalytic.

In a third embodiment of the fifth aspect, the target is another polypeptide. Polypeptides, including proteins, that find use herein as targets for binding ligands, such as, for example small organic molecule ligands, include virtually any polypeptide (including short polypeptides also referred to as peptides) or proteins that comprise two or more binding sites of interest. Polypeptides of interest may be obtained commercially, recombinantly, by chemical synthesis, by purification from natural source or other approaches known to those of skill in the art. In another embodiment, one or more polypeptides may be substantially purified.

In a fourth embodiment of the fifth aspect, hits obtained from screening are screened against another biological molecule of interest to ascertain differences in an affinity parameter of the hits for the target as against the another biological molecule. In an embodiment, the hits are closely related to the biological molecule. Such screens may be referred to as counterscreens, and the other biological molecule may be referred to as an anti-target.

In a fifth embodiment of the fifth aspect, a spatially addressed array of vessels is provided as at least one multi-well plate. In one embodiment, a 96-well plate is used. In another embodiment, a 394-well plate is used. In another embodiment, a 1536-well plate is used. In another embodiment, a microfluidic device is used. One of skill in the art can easily identify the appropriate size plate or device to use for the size of the library to be employed.

In an embodiment of any of the aspects, synthesized polypeptides may be substantially purified.

Also provided herein are libraries containing a plurality of mRNA-tRNA polypeptide complexes, the plurality containing mRNA-tRNA-polypeptide complexes that differ from one another, e.g., wherein the mRNA of each mRNA-tRNA-polypeptide complex encodes a different polypeptide. The libraries of the invention may be prepared according to any of the aspects or embodiments as described herein.

The methods of the present invention may be used to synthesize a wide variety of chemical compounds. In certain embodiments, the methods are used to synthesize and evolve unnatural polymers (i.e., excluding polypeptides), which cannot be amplified and evolved using standard techniques currently available. In certain other embodiments, the inventive methods and compositions are utilized for the synthesis of small molecules that are not typically polymeric. In still other embodiments, the method is utilized for the generation of non-natural polymers.

### BRIEF DESCRIPTION OF THE DRAWINGS

FIG. 1 shows a schematic diagram of the structure and topology of the ribosome, including the small subunit (left), large subunit (right) and assembled ribosome (center). The figure shows the location of some important functions of the ribosome, such as the Decoding center (left), the Peptidyl Transferase center (right) as well as the placement of messenger RNA in the mRNA tunnel, the peptidyl-tRNA, and the synthesized peptide leaving the Exit Tunnel (center). FIG. 1 also indicates the relative placement of the A, P and E sites, with the A site being the point of entry for aminoacyl-tRNA, the P-site being the place where peptidyl-tRNA is formed in the ribosome, and the E-site being the exit site of the deacylated tRNA.

FIG. 2 illustrates relevant structures of aminoacyl-tRNAs showing an attached acyl group on the 3′ terminal adenosine of tRNA. FIG. 2A shows the chemical structure of an aminoacyl-tRNA with a side chain R. FIG. 2B shows the secondary structure and sequence (SEQ ID NO: 1) of an engineered tRNA_{CUA}^{Phe} from yeast, with a 3′ CCA aminoacyl acceptor stem and a CUA anticodon that reads a selector codon UAG, for genetic encoding of the amino acid side chain.

FIG. 3 shows structural representations of aminoacyl-tRNA ligand adduct moieties. FIG. 3A shows a ligand adduct moiety with a linker, z, that is formed from the Huisgen [3+2] cycloaddition reaction of p-azidophenylalanine, A, acyl-tRNA with an alkyne ligand, L. FIG. 3B shows a general representation of an aminoacyl-tRNA non-canonical amino acid ligand adduct having the structure tRNA-A-z-L, as described according to the invention.

FIG. 4 is an illustration of the plasmid map of pGB014 DNA vector used for T7 RNA polymerase catalyzed transcription of an Methanococcus jannaschii (Mj) amber suppressor tRNA sequence (Albayrak, C. and Swartz, J. R., Nucleic Acids Research (2013) 41:5949-5963) showing relevant sites on the plasmid vector, including (i) a T7 promoter sequence operably linked to the sequence coding for the tRNA with (ii) a CUA anti-codon, and (iii) 3′ terminal CCA aminoacyl acceptor stem, (iv) an HDV ribozyme sequence, (v) an RNA spacer sequence, and (vi) 3′ stem-loop transcription terminators.

FIG. 5 illustrates the steps in an exemplary method for generating an aminoacyl-tRNA with an orthogonally reactive moiety y (in this case, p-azido-L-phenylalanine), including the steps of (A) in vitro transcription of a tRNA-HDV ribozyme fusion DNA template, (B) enzymatic hydrolysis of tRNA 2,3′-cyclic phosphate using T4 polynucleotide kinase (PNK), (C) purification of tRNA by strong anion-exchange (IEX) chromatography, and (D) aminoacylation of adapter tRNA with a non-canonical amino acid with an orthogonally reactive moiety (in this case, an azido group), catalyzed by an engineered aaRS enzyme. See United States patent application Voloshin, A. M., et al., US20100184135 (2009), incorporated herein as reference.

FIG. 6 shows (A) FPLC anion exchange (IEX) chromatogram showing A260 vs time for purification of a transcription reaction of pGB014 to form Mj tRNA_{CUA}^{Tyr} 3′ cyclic phosphate as described in Example 1 set forth below. (B) TBE-UREA 10% PAGE gel analysis of individual fractions. The indicated fractions of Mj tRNA_{CUA}^{Tyr} were pooled, concentrated, and subsequently analyzed by (C) hydrophobic interaction chromatography (HIC) using a 25 cm C5 HPLC column monitored at 260 nm, eluted with a gradient from 1.5 M ammonium sulfate (A) to 5% isoproponal in (B) 50 mM potassium phosphate, pH 5.7, at a flow rate of 0.3 ml/min over 50 min.

FIG. 7 is an illustration of the plasmid map of pGB028A vector, used for T7 RNA polymerase catalyzed transcription of an Escherichi coli (Ec) amber suppressor tRNA sequence showing relevant sites on the plasmid vector, including (i) a T7 promoter sequence operably linked to the sequence coding for the tRNA with (ii) a CUA anti-codon, and (iii) an HDV ribozyme sequence, (iv) an RNA spacer sequence, \and (v) a 3′ stem-loop transcription terminator.

FIG. 8 shows (A) FPLC chromatogram A260 vs time for purification of Ec Met-tRNA_{CUA}^{Met} by anion exchange chromatography and (B) TBE-UREA 10% PAGE gel of individual fractions. The indicated fractions of purified Ec tRNA_{CUA}^{Met} were pooled and concentrated as described in Example 2 set forth below.

FIG. 9 is a photograph showing Ni²⁺-NTA purified six-histidine (6×His)-tagged tRNA synthetase (RS) enzyme variants as assayed by SDS-PAGE (polyacrylamide gel electrophoresis) as described in Example 3 set forth below. Lane 1: Molecular Weight marker. Lane 2: Molecular Weight marker. A six-histidine-tag was added to the COOH terminus of M. jannaschii Tyrosyl tRNA synthetase variants shown in lane 3 (WT), lane 4 (E3 variant), lane 5 (E11 variant), lane 6 pCNPhe TyrRS variant. A 6×His tag was added to the NH₂ terminus of Desulfitobacterium hafniense Pyrolysl tRNA synthetase (Dh Pyl RS) in Lane 8, and an E. coli methionine RS variant in Lane 9. The gel was stained with coomassie blue. The expected molecular weights of the enzyme variants are shown on the right, in kD.

FIG. 10 illustrates the design and screening of E. coli methionine tRNA synthetase (RS) variants for efficient aminoacylation of tRNA with non-canonical amino acids containing orthogonally reactive moieties. FIG. 10A. shows the X-ray structure (PDB accession no. 1F4L) showing location of active site residues to be mutated, along with UAG suppressor mutations able to recognize E. coli tRNA_{CUA}^{Met} (●. FIG. 10B. shows the X-ray structure of tRNA^{Met}, with the RNA backbone and base-pairs within the X-ray structure. FIG. 10C depicts representative amino acids with side chains containing orthogonally reactive alkyne, alkene, and azide moieties A-y for linking with reactive ligand moieties L-x.

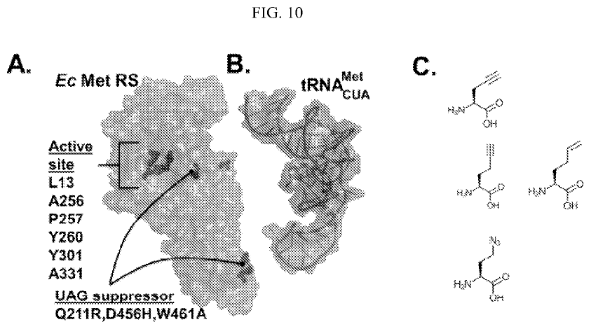

FIG. 11 illustrates hydrophobic interaction chromatography (HIC) analysis of purified Mj tRNA_{CUA}^{Tyr} using a C5 HPLC column monitored at 260 nm, eluted with a gradient from 1.5 M ammonium sulfate (A) to 5% isopropanol (B) in 50 mM potassium phosphate buffer, pH 5.7 at a flow rate of 0.3 ml/min over 50 min.

FIG. 12 illustrates non-canonical 3-fluoro tyrosine and 2,3-difluoro tyrosine (Fₙ-Tyr) aminoacyl-tRNA_{CUA}^{Tyr} produced in vitro by enzymatic charging with engineered tRNA synthetase (RS) enzymes as set forth in Examples 3 & 4 below, analyzed by hydrophobic interaction chromatography (HIC)-HPLC.

FIG. 13 shows a representative electrophoretogram by capillary zone electrophoresis of Methanococcus jannaschii (Mj) amber suppressor tRNA (Albayrak, C. and Swartz, J. R., Nucleic Acids Research (2013) 41:5949-5963) produce by the methods described in Example 1 below, using a G1600 Agilent instrument and a 50 μm×72 cm untreated fused-silica capillary from Agilent. Voltage was set at 30 kV, the buffer was 50 mM borate, the pH was 9.3, and the samples were monitored at 260 nm.

FIG. 14 shows a time course for aminoacylation of 25 μM Methanococcus jannaschii (Mj) amber suppressor tRNA by 25 μM Mj TyrRS as set forth in Example 6 below. Aminoacyl-tRNA samples were quenched at various time points with 1/10^{th} volume 3M Acetic Acid, pH 5.9, phenol-chloroform extracted, and purified by size-exclusion using a Bio-Spin column treatment. Time point samples were mixed with 50 μl of buffer A (50 mM potassium phosphate, 1.5 M ammonium sulfate, pH 5.7) and the extent of aminoacylation monitored at 260 nm using HIC-HPLC. The relative amounts of tRNA and aminoacyl-tRNA were determined by peak height. Inset: First order kinetic analysis of the extent of aminoacylation (ca. 86%).

FIG. 15 shows overlaid HIC-HPLC chromatograms, normalized to the peak height of Mj tRNA_{CUA}^{Tyr} (●●●●), and several aminoacylated Mj tRNA_{CUA}^{Tyr} variants prepared according to the methods described in Examples 4, 5, and 6. The extent of aminoacylation (in paraentheses) was determined by comparing the peak height of the aminoacyl product to the peak height of tRNA: tyrosine (88%), p-methoxyphenylanine (70%), p-azido-phenylalanine (64%), p-t-butylphenylalanine (83%), and biphenylalanine aminoacyl-tRNAs (73%).

FIG. 16 shows a plot of the retention times of aminoacyl-tRNA variants, relative to the retention time of tRNA_{CUA}^{Tyr} on HIC-HPLC versus the polarity index (log P) of the corresponding amino acid, as calculated (Livingstone, D. J., Curr Top Med Chem (2003) 3:1171-1192) by their octanol/water partitioning coefficients.

FIG. 17 shows overlaid HIC-HPLC chromatograms of Mj tRNA_{CUA}^{Tyr} (●●●●) and pAzPhe-tRNA_{CUA}^{Tyr} reacted with propargyl alcohol under standard click-chemistry conditions as described in Example 9.

FIG. 18 shows overlaid HIC-HPLC chromatograms of Mj tRNA_{CUA}^{Tyr} and pAzPhe-tRNA_{CUA}^{Tyr} before and after reaction of the azide functional group with propargyl benzoate under standard click-chemistry conditions. The yield of propargyl benzoate reacted aminoacyl-tRNA analogue is 64%, relative to unreacted pAzPhe-tRNA_{CUA}^{Tyr}.

FIG. 19 shows the HIC-HPLC chromatogram of pAcPhe-tRNA_{CUA}^{Tyr} before and after reaction of the para-acetyl functional group with alpha-effect nucleophiles hydroxylamine, methoxyamine, and O-(2-(Vinyloxy)ethyl)hydroxylamine. The tRNA ligand adducts formed by oxime ligation as described in Example 10 below show almost quantitative conversion, although a fraction of the starting aminoacyl-tRNA is also cleaved by the added alpha-effect nucleophiles. The tRNA ligand adduct yields are 95% for hydroxylamine and methoxyamine adducts and 85% for the O-(2-(Vinyloxy)ethyl)hydroxylamine tRNA adduct, relative to unreacted pAcPhe-tRNA_{CUA}^{Tyr}.

FIG. 20 shows amino acid side chain polarity on the x-axis and size on the y-axis for aminoacyl-tRNA structures capable of ribosome-directed translation. The characteristics of amino acid side chains are plotted as a function of polarity (on the x-axis, log P partitioning coefficient between octanol/water) and side chain volume (on the y-axis, van der Waals volume, A³). Shown are (i) the 20 canonical amino acids (α), indicated by the 1-letter amino acid code, (ii) representative literature examples of non-canonical amino acids incorporated into proteins (6), and (iii) representative examples of triazole ligand adducts of the present invention (▪) incorporated into proteins by in vitro ribosome-directed translation. The inset shows a representative phenylalanine amino acid-triazole ligand adduct structure, cf. FIG. 3A.

FIG. 21 shows how the EF-Tu protein is engineered for efficient ribosome-directed phospho-threonine (p-Thr) incorporation into polypeptides. FIG. 21A shows residues lining the amino acid binding pocket of E. coli EF-Tu complexed with Phe-tRNA^{Phe} (from PDB file 10B2). The chart in FIG. 21B illustrates mutations in these residues in E. coli EF-Tu and variants EF-Sep and EF-Sep21 that recognize phospho-Ser-tRNA (Lee, S., et al., Angew Chem Int Ed Engl (2013) 52:5771-5775). These serve as a starting point to design an alanine scan to identify recognition hot-spots for interaction with phospho-Thr-tRNA.

FIG. 22 shows, as described in Example 13 below, (A) growth curves for several E. coli strains engineered for use as extracts in cell-free protein synthesis and (B) their corresponding doubling times, in minutes when grown in shake flasks in 2×YT media at 280 rpm at 37° C.

FIG. 23 shows a cell-free transcription-translation reaction of superfolder GFP as described in Example 15 below. A well suppressed Q157TAG super-folder GFP variant was used to measure protein expression by fluorescence, using UAG suppression with various concentrations of purified Mj tRNA_{CUA}^{Tyr} 3′ cyclic phosphate (cf. FIG. 11) or the corresponding tRNA-HDV ribozyme fusion plasmid, cf. FIG. 4, in the presence of 25 Mj TyrRS enzyme and T4 PNK. There is sufficient phosphatase activity in the lysate to activate the added tRNA for aminoacylation and subsequent translational incorporation of Tyr at the UAG codon.

FIG. 24 shows a cell-free transcription-translation reaction of superfolder GFP as described in Example 16 below. A well suppressed superfolder GFP variant Q157TAG (▪) was used to measure protein expression by fluorescence, using UAG suppression in the presence of various concentrations of aminoacyl pAz-Phe-tRNA_{CUA}^{Tyr} (▪) (cf. FIG. 15) or the corresponding unaminoacyleted tRNA_{CUA}^{Tyr} (▪).

FIG. 25 shows a functional transcription-translation assay evaluating Sc pAzPhe-tRNA_{CUA}^{Phe}. A Q157TAG super-folder GFP (sfGFP) variant was used to measure protein expression by fluorescence, using UAG suppression with the addition of various concentrations of Sc tRNA_{CUA}^{Phe}. Addition of Sc tRNA_{CUA}^{Phe} alone does not suppress above background.

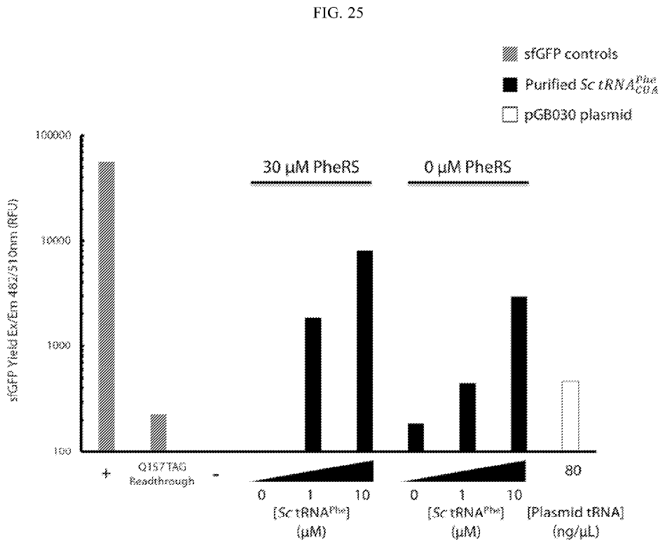

FIG. 26 shows representative examples of reactive moieties x and y, and the chemical structures of the products formed. The following reactive moieties are set forth: a) an azide and an alkyne; (b) an alkene and a thiol or an amine (not shown); (c) a vinyl sulfone and a thiol or an amine (not shown); (d) an α-halo-carbonyl and a thiol or an amine (not shown); (e) a disulfide and a thiol; (f) a carbonyl and an alpha-effect amine; (g) an activated carboxylic acid and a alkyl or aryl (not shown) amine; (h) a 1,4-dicarbonyl and an alkyl or aryl (not shown) amine; (i) an aryl halide and an alkyl boronate ester, and (j) an aryl halide and an alkyne.

FIG. 27 illustrates the synthesis of alpha-hydroxy acid-tRNA, wherein L is a ligand as described herein, z is a covalent linker, as described herein, and A is an aminoacyl group attached to tRNA (left) reacted to form a hydroxyacyl group (right) in the presence of NaNO₂.

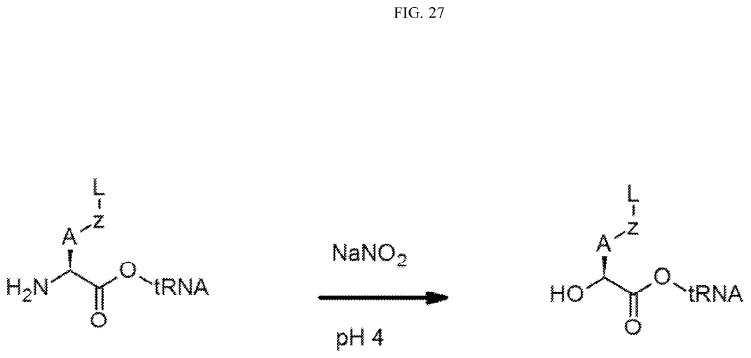

FIG. 28 shows a representative scheme for producing a large spatially addressed library of aminoacyl-tRNA non-canonical amino acid ligand adducts according to the present invention. FIG. 28A describes representative engineered aaRS enzyme variants, FIG. 28B shows non-canonical amino acids with reactive moieties (A-y) set onto the 3′ aminoacyl acceptor stem of FIG. 28C amber & opal suppressor tRNAs (tRNA_{CUA} and/or tRNA_{UCA} (tRNAₘ)) to form in FIG. 28D acyl-tRNAs (tRNAₘ-A-y). The subsequent reaction of these preselected acyl-tRNAs with preselected ligand library members containing reactive moieties (x-Lₙ), FIG. 28E, purified in arrayed format, yields FIG. 28F, a spatially addressed library of aminoacyl-tRNA non-canonical amino acid ligand adducts (tRNAₘ-A-z-Lₙ) with amber and/or opal suppressor tRNAs.

FIG. 29 shows representative ligand structures with reactive moieties, x-Lₙ, of the present invention. Chiral centers are indicated by *. FIG. 29A shows unbiased ligands with reactive moieties and FIG. 29B shows Bcl-2 protein target focused ligands with reactive moieties are shown. FIG. 29C shows an example synthetic strategy for synthesis of a specific library member.

FIG. 30 shows how the spatial relationship between ligand adduct moieties may be decoded from the polypeptide sequence, based on known rules of protein folding and secondary structure of Bacillus lichenformis beta-lactamase (FIG. 30A). Bacillus lichenformis beta-lactamase consists of an active site for hydrolysis of beta-lactam substrates such as fluorocillin, and structurally conserved alphahelices at the N- and C-terminus. An N-terminal extension consisting of a single alpha-helical turn (residues ²⁷AEF³⁰A, illustrated in FIG. 30B as an alphahelica wheel) was fused to a streptavidin binding peptide sequence (SBP-Tag2). After translation of an A34TAG variant in the presence and absence of Mj pCNF RS/tRNA_{CUA}/ncAAs, enzyme variants were pulled down on strepativdin plates and assayed for beta-lactam hydrolysis of fluorocillin, FIG. 30C. Enzyme-catalyzed fluorescence activity indicates folded and functional beta-lactamase was formed.

FIG. 31 shows selection and evolution of encoded ligand adduct libraries as described in Example 19 below. As shown, a library of ligand adduct-tRNAs (a) is translated by the ribosome using encoded mRNAs, (b) to produce spatially addressed ribosome displayed polypeptide complexes, (c). The mRNAs may contain a predetermined barcode which is uniquely associated with a non-canonical amino acid. The unique association may be used to identify polypeptide products of the translation system. The predetermined barcode in FIG. 31 is shown as exemplary Seq IDs 19, 13 and 1. These sequences are exemplary only. The ribosome displayed ligand adduct libraries are pooled (c′), selected for specific binding to a target, (d), and the encoded mRNAs recovered and amplified by RT-PCR. The pooled PCR product may be sequenced, (e), to decode the chemical structures of selective binders. This information may be subsequently used to design new ligand adduct-tRNA libraries for additional rounds of selection and screening.

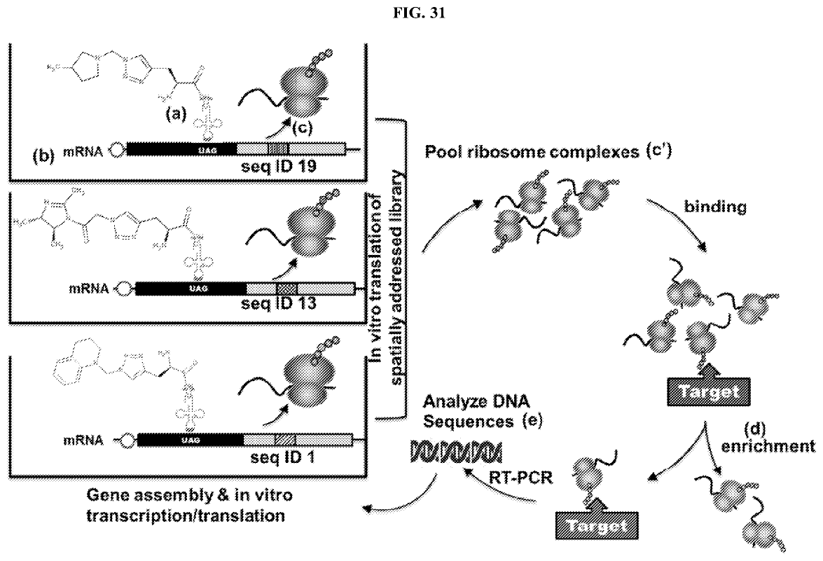

FIG. 32 illustrates the generation of a DNA encoded library of alpha-helical polypeptides with non-canonical amino acid ligand adducts, for inhibition of alpha-helix mediated protein protein interactions. Amino acid residues at positions i, i+4, and i+7 form a single face of an alpha-helical polypeptide. A DNA encoded library of diverse ligand adducts displayed across the face of an alpha-helix is designed using the diversity code rules shown. Spatially addressed encoded libraries can be then be transcribed and translated using suppressor tRNAs (tRNA_{CUA} and tRNA_{UCA}) aminoacylated with ligand adducts, tRNAₘ-A-y-Lₙ, for example.

FIG. 33 shows the design of ribosome display selections for ligand adduct trapping as described in Example 19 below. (A) Gene sequence cassette design indicating T7 promoter, V_{H}-linker-V_{L} scFv antibody sequence fused to a ribosome display spacer sequence. The genetically encoded FₙY residue at position 37 in V_{L} is coded by TAG and library diversity in the V_{H} and V_{L} CDRs are generated by designed synthetic oligos (Integrated DNA Technologies, Inc.). (B) Assembly of naïve scFv V_{H} and V_{L} CDR library into a transcription/translation ready PCR cassette (1227 bp) for ribosome display using overlap PCR c.f. Stafford et al (2014). Gel is 1% agarose visualized under UV light with 1× GelRed nucleic acid dye (Biotium). (C) Schematic illustrating the steps of ribosome display. Displayed scFv clones covalently capture the biotinylated hapten and are separated from non-bound scFv by magnetic bead pull-down. Captured scFv/hapten complexes are released by hydroxylamine cleavage of the tyrosyl sulfonate. (D) Proof-of-concept demonstration of wildtype A.17 scFv pulldown on streptavidin beads in the presence or absence of the biotinylated hapten. The more intense band at 1227 bp in the presence of hapten indicates positive selection.

FIG. 34 shows the output of ribosome display selections for ligand adduct trapping as described in Example 19 below. (A) Library DNA was recovered by RT-PCR following each round of selection pressure and visualized by agarose gel electrophoresis. The more intense band in the presence of hapten following selection round 5 indicated library enrichment. Recovered DNA from the fifth round of selection was subcloned into an expression vector, transformed into E. coli, and DNA sequences of individual colonies were obtained by Sanger sequencing. There was no sequence consensus for the scFV CDR-H3 (B) or CDR-L3 (C). Positions within the A.17 scFv CDR that were randomized are indicated by a star.

FIG. 35 shows (A) X-ray structure of the RPA N-terminal domain bound to p53 transactivation domain (residues 47-57). A 120° rotation illustrates the hydrophobic cluster of p53 residues that define the binding epitopes for this target Protein-Protein Interaction. (B). X-ray structure of a 13-amino acid FITC-labeled peptide-33 (Kd=22 nM) with a 3,4-dichlorophenyl side chain that binds the same hydrophobic pocket of RPA as the F54 residue in the p53 peptide. (C) Relative yields of beta-lactamase (BLA) N-terminal alpha-helix variants with nnAAs incorporated at UAG & UGA codons, as monitored by enzyme activity. This shows that alpha-helical proteins containing ligand adduct moieties can be expressed by cell-free protein synthesis. (D) Alpha-helical wheel representation of an N-terminal BLA-fusion library based on SAR (Frank, A. O. et al. Journal of Medicinal Chemistry (2013) 56, 9242-9250). The BLA scaffold on one face of the α-helix is shown. This library of alpha-helically displayed ligand adducts is selected using the methods described herein.

### DETAILED DESCRIPTION OF THE INVENTION

### Definitions

As used herein, the term “peptide”, “peptides”, “protein”, or “proteins” means polypeptide molecules formed from linking various amino acids in a defined order. The link between one amino acid residue and the next forms a bond, including, but not limited to, an amide or peptide bond, or any other bond that can be used to join amino acids. The peptides/proteins may include any polypeptides of two or more amino acid residues. The peptides/proteins may include any polypeptides including, but not limited to, ribosomal peptides and non-ribosomal peptides. The peptides/proteins may include natural and unnatural amino acid residues. The number of amino acid residues optionally includes, but is not limited to, at least 5, 10, 25, 50, 100, 200, 500, 1,000, 2,000 or 5,000 amino acid residues. The number of amino acid residues optionally includes, but is not limited to, 2 to 2,000, 2 to 1,000, 2 to 500, 2 to 250, 2 to 100, 2 to 50, 2 to 25, 2 to 10, 5 to 2,000, 5 to 1,000, 5 to 500, 5 to 250, 5 to 100, 5 to 50, 10 to 2,000, 10 to 1,000, 10 to 500, 10 to 250, 10 to 100, or 10 to 50.

As used herein, the term “amino acid” or “amino acids” means any molecule that contains both amino and carboxylic acid functional groups, including, but not limited to, alpha amino acids in which the amino and carboxylate functionalities are attached to the same carbon, the so-called α-carbon. Amino acids may include natural amino acids, unnatural amino acids, and arbitrary amino acids. Amino acids may include N-alkyl amino acids, α,α-disubstituted amino acids, β-amino acids, and D-amino acids.

As used herein, the term “natural amino acid” or “canonical amino acid” or “canonical aminoacyl” includes, but is not limited to, one or more of the amino acids encoded by the genetic code. The genetic codes of all known organisms encode the same 20 amino acid building blocks with the rare exception of selenocysteine and pyrrolysine (Xie, J. and Schultz, P. G., Methods (2005) 36:227-238). In some embodiments, natural amino acids may also include, but not be limited to, any one or more of the amino acids found in nature. In some embodiments, these natural amino acids may include, but not be limited to, amino acids from one or more of plants, microorganisms, prokaryotes, eukaryotes, protozoa or bacteria. In some embodiments, natural amino acids may include, but are not limited to, amino acids from one or more of mammals, yeast, Escherichia coli, or humans.

As used herein, the term “non-canonical amino acid” or “non-natural amino acid” may include any amino acid other than the natural amino acids encoded by the genetic code known to those of skill in the art. In some embodiments, non-canonical amino acids may include, but not be limited to, modified or derivatized canonical amino acid encoded by the genetic code or any one or more of the amino acids found in nature. In some embodiments, non-natural amino acids may include, but not be limited to, modified or derivatized amino acids from one or more of plants, microorganisms, prokaryotes, eukaryotes, protozoa, bacteria, mammals, yeast, Escherichia coli, or humans. In some embodiments, non-canonical mino acids may include N-alkyl amino acids, α,α-disubstituted amino acids, β-amino acids, and D-amino acids.

As used herein, the term “amino acid residue” or “amino acid residues” means the remainder of an amino acid incorporated into a peptide/protein.

As used herein, the term “side chains” of amino acids refers to a moiety attached to the α-carbon (or another backbone atom) in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for p-azido-phenylalanine is 4-azidophenylmethyl, etc. Other non-naturally occurring amino acid side chains are also included, for example an α-α di-substituted amino acid, a beta-amino acid, or an N-alkyl amino acid.

As used herein, the term “RNA” is meant a sequence of two or more covalently bonded, naturally occurring or modified or derivatized ribonucleotides.

As used herein, the term “tRNA”, “tRNAs”, “transfer RNA”, or “transfer RNAs” means an RNA chain that transfers an amino acid to a growing polypeptide chain on a ribosome. The tRNA has a 3′ aminoacyl acceptor stem for amino acid attachment and an anti-codon loop for selector codon recognition. In some embodiments, tRNA includes natural, unnatural, and synthetic tRNA. The aminoacyl acceptor stem includes a 3′ terminal cytosine cytosine adenosine ribonucleotide sequence, which by convention is designated C74, C75 and A76, and which covalently binds to the amino acid it carries via an acyl linkage on the 3′ terminal adenosine.

As used herein, the term “natural tRNA” means one or more tRNA known in nature that transfer an amino acid to a growing polypeptide chain. In some embodiments, natural tRNA includes, but is not limited to, tRNA that transfer one or more of the natural amino acids that are encoded by the genetic code. In some embodiments, natural tRNA include, but are not limited to, natural tRNA from one or more of plants, microorganisms, prokaryotes, eukaryotes, protozoa, bacteria, mammals, yeast, E. coli, humans, or archaea. In some embodiments, natural tRNA is post-transcriptionally modified.

As used herein, the term “unnatural tRNA” means any tRNA, other than tRNA known in nature, which transfers an amino acid to a growing polypeptide chain. In some embodiments, unnatural tRNA may include, but not be limited to, modified or derivatized natural tRNA. In some embodiments, unnatural tRNA may include, but not be limited to, modified or derivatized natural tRNA from one or more of plants, microorganisms, prokaryotes, eukaryotes, protozoa, bacteria, mammals, yeast, Escherichia coli, humans, or archae. In some embodiments, unnatural tRNA may include, but not be limited to, tRNA with altered sites for amino acid attachment, and/or tRNA with altered acceptor stems, and/or tRNA with altered sites for codon recognition (the anti-codon). In some embodiments, unnatural tRNA is recombinant tRNA. In some embodiments, unnatural tRNA displays reduced ability to act as a donor substrates or acceptor substrate for ribosome-directed translation. In some embodiments, unnatural tRNA may be modified to include modified nucleosides, for example, pseudouridine (ψ), 5-methylcytidine (m5C), N6-methyladenosine (m6A), 5-methyluridine (m5U), 2-thiouridine (s2U), phosphothioate linkages, or 2′ deoxycytosine (dC) cf. Hou, Y.-M., Recombinant and In Vitro RNA Synthesis (2012) 941:195-212.

As used herein, the term “arbitrary tRNA” means a tRNA that has been modified or derivatized such that the amino acid attachment site may bind one or more amino acids other than the amino acid specified by the anti-codon based on the genetic code. The amino acid may be natural or non-natural. Arbitrary tRNA may also include tRNAs that have been modified or derivatized such that the amino acid attachment site may bind one or more different amino acids (natural or non-natural), while the anti-codon may recognize one or more of one or more stop codons, or one or more selector codons. DNA stop codons may include ochre (TAA), amber (TAG), and opal (TGA). The corresponding arbitrary tRNA anti-codons in suppressor tRNAs are: ochre suppressor tRNA_{UUA}, amber suppressor tRNA_{CUA}, or opal suppressor tRNA_{UCA}.

As used herein, the term “anti-codon” represents a sequence of at least three adjacent nucleotides in transfer RNA that bind to a corresponding selector codon in messenger RNA during ribosome-directed translation, and designates a specific amino acid or α-hydroxyl acid during ribosome-directed translation.

As used herein, the term “selector codon” represents a sequence of at least three adjacent nucleotides in messenger RNA that binds to a corresponding anti-codon in tRNA and designates a specific amino acid or α-hydroxyl acid during ribosome-directed translation. Selector codons may include 4-base, 5-base, even 6-base codons.

As used herein, the term “acylated tRNA” or “acyl-tRNA” or “aminoacyl-tRNA” refers to tRNA that has an ester bond at a 3′ hydroxyl of the ribose. During peptide synthesis, the aminooacyl group is transferred to the nascent peptide, releasing the tRNA. In some embodiments, the aminoacyl-tRNA may be natural, unnatural and/or arbitrary. In some embodiments acylated tRNA may be modified at the amino group of acyl-tRNAs for example by N-methylation, N-alkylation, and/or with reversible protecting groups.

As used herein, the term “released tRNA” or “unacylated tRNA” means the tRNA remaining after the aminoacyl-tRNA has donated the attached amino acid to the nascent polypeptide.

As used herein, the term “mRNA”, “mRNAs”, or “messenger RNA” means an RNA sequence that directs the synthesis of, or is operably linked to, a second molecule by action of the ribosome. The mRNA sequence may encode a biopolymer sequence via interaction of a selector codon with an anti-codon on an arbitrary acylated-tRNA or by linking the translated product to the encoded message that is translated. The messenger RNA may consist of modified RNA with modified nucleosides, for example, pseudouridine (ψ), 5-methylcytidine (m5C), N6-methyladenosine (m6A), 5-methyluridine (m5U), 2-thiouridine (s2U), or 2-deoxycytosine. The mRNA may include RNA sequences such as 5′ and 3′ untranslated regions (UTR) and/or ribosomal binding sequences (RBS), for example, a Shine-Dalgarno sequence, operably linked to a promoter sequence. The RBS allows ribosomes to bind to and initiate translation of the mRNA into a polypeptide. The 5′ and 3′ UTRs can also include stabilizing stem loops to protect the mRNA from exonuclease degradation. The mRNA sequence may be natural or may be optimized for translation by methods well known in the art cf. Hellinga, H., et al, United States Patent Application 2011/0171737, incorporated herein in its entirety and Li, G. W., et al., Nature (2012) 484:538-541. The mRNA sequence may contain a barcode sequence that is coding or non-coding. In some embodiments, the mRNA may contain a ribosome trapping sequence, generally in the range of from 60-900, 90-300, or 120-240 nucleotides in length. The ribosome trapping sequence can comprise from about 10-1000, 20-500, or 30-100 codons that are in the same reading frame as the codons in the ORF. The ribosome trapping sequence, sometimes referred to as a spacer or tether sequence functions to tether the ribosome to the mRNA template, thereby allowing the translated polypeptide to emerge from the ribosome tunnel and either fold into a tertiary structure, or extend some distance outside of the ribosome attached to an unstructured polymer sequence such as recombinant PEG sequences. In some embodiments, the ribosome trapping sequence does not contain a stop codon. The mRNA may be covalently linked at the 3′ end to a non-RNA pause sequence.

As used herein, the term “transfer-messenger RNA” or “tmRNA” refers to an RNA molecule with dual tRNA-like and messenger RNA-like properties. The tmRNA forms a ribonucleoprotein complex (tmRNP) together with Small Protein B (SmpB) and Elongation Factor Tu (EF-Tu). In trans-translation, tmRNA and its associated proteins bind to bacterial ribosomes which have stalled during protein biosynthesis, for example when reaching the end of a messenger RNA which has lost its stop codon. The tRNA-like domain of tmRNA contains a D-loop, a T-arm and an aminoacyl acceptor stem CCA competent to form an aminoacyl-tmRNA. In some embodiments, the aminoacyl-tmRNA may be natural, unnatural and/or arbitrary.

As used herein, the term “arbitrary tmRNA” means a tmRNA that has been modified or derivatized such that the amino acid attachment site may bind one or more amino acids other than the amino acid specified by the tmRNA determinants. The amino acid may be natural or non-natural. Arbitrary tmRNA may also include tmRNAs that have been modified or derivatized such that the amino acid attachment site may bind one or more different amino acids (natural or non-natural), while the tmRNA may recognize one or more aminoacyl tRNA synthetases.

As used herein, the term “acylation” or “charging” is a process of adding an acyl group to a compound. Methods for charging natural, unnatural and/or arbitrary tRNA and natural, unnatural and/or arbitrary tmRNAs with natural, non-natural and/or arbitrary amino acids are known in the art, and include, but are not limited to, chemical aminoacylation, biological misacylation, acylation by natural and engineered aminoacyl-tRNA synthetases, ribozyme-based, and protein nucleic acid-mediated methods Hecht, S. M., Protein Engineering (2009) 22:255-270; Xie, J. and Schultz, P. G., Methods (2005) 36:227-238; Kourouklis, D., et al., Methods (2005) 36:239-244; Tan, Z., et al., Methods (2005) 36:279-290.

As used herein, the term “aminoacyl-tRNA synthetase” or “aaRS” means an enzyme or ribozyme that catalyzes the binding of one or more amino acids or α-hydroxyl acids to a tRNA to form an aminoacyl-tRNA or α-hydroxylacyl-tRNA. In some embodiments, the synthetase binds the appropriate amino acid to one or more tRNA molecules. In some embodiments, the synthetase mediates a proofreading reaction to ensure high fidelity of tRNA charging. In some embodiments, the synthetase does not mediate a proofreading reaction to ensure high fidelity of tRNA charging.

As used herein, the term “natural aminoacyl-tRNA synthetases” means aminoacyl-tRNA synthetases known in nature that add an aminoacyl group to a tRNA. In some embodiments, natural aminoacyl-tRNA synthetases include, but are not limited to, aminoacyl-tRNA synthetases that add one or more of the natural aminoacyl groups that are encoded by the genetic code. In some embodiments, natural aminoacyl-tRNA synthetases include, but are not limited to, natural aminoacyl-tRNA synthetases from one or more of plants, microorganisms, prokaryotes, eukaryotes, protozoa, bacteria, mammals, yeast, Escherichia coli, or humans.

The term “engineered aminoacyl-tRNA synthetase” means any aminoacyl-tRNA synthetase, other than aminoacyl-tRNA synthetases known in nature that add an aminoacyl or α-hydroxylacyl group to a tRNA. In some embodiments, engineered aminoacyl-tRNA synthetases may include, but are not limited to, modified or derivatized natural aminoacyl-tRNA synthetases. In some embodiments, engineered aminoacyl-tRNA synthetases may include, but are not limited to, modified or derivatized natural aminoacyl-tRNA synthetases from one or more of plants, animals, microorganisms, prokaryotes, eukaryotes, protozoa, bacteria, mammals, yeast, E. coli, or humans. In some embodiments, engineered aminoacyl-tRNA synthetases may include, but are not limited to, aminoacyl-tRNA synthetases with altered aminoacyl specificity and/or altered tRNA specificity, and/or altered editing ability.

As used herein, the term “altered specificity” means that the specificity typically observed in nature has been changed. In some embodiments, altered specificity includes, but is not limited to, broadening the specificity to include, for example, recognition of additional amino acids, α-hydroxyl acids, and/or additional tRNA. In some embodiments, altered specificity includes, but is not limited to, changing the identity of the aminoacyl group and/or tRNA from the aminoacyl group and/or tRNA recognized in nature. In some embodiments, altered specificity may be measured by k_{cat}/Kₘ for aminoacylation of tRNA and may be equal to or higher than k_{cat}/Kₘ for that observed in nature.

As used herein, the term “cell-free lysate” refers to preparation of in vitro reaction mixtures able to translate mRNA into polypeptides. The mixtures include ribosomes, ATP, amino acids, and tRNAs and other factors. They may be derived directly from lysed bacteria, from purified components, or combinations of both. See for example, patent application Voloshin, A. M., et al., US20100184135 (2009) (incorporated herein by reference in its entirety).

As used herein, “ribosome” means biological ribonucleoproteins that serve as the primary site for translation and include, but are not limited to, one or more ribosomes. The ribosomes may be one or more of eukaryotic ribosomes and/or prokaryotic ribosomes. In some embodiments, the ribosomes are partially or completely isolated, purified, or separated from cells, other cellular material, and/or tissues. In some cases the ribosomes are produced in vitro. In some embodiments, the ribosomes are from mitochondria and/or chloroplasts. The ribosomes may be from one or more of plants, animals, microorganisms, prokaryotes, eukaryotes, protozoa, bacteria, mammals, yeast, E. coli, and/or humans. In some embodiments the ribosomal RNA or protein sequence is engineered. The ribosome small subunit is responsible for the decoding process whereby aminoacyl tRNA is selected according to the selector codon. Its major functional sites are the mRNA path used to conduct mRNA during translation, the decoding center responsible for decoding, and the tRNA binding sites (A, P and E). The ribosome large subunit catalyzes peptide bond formation. Its major functional sites are the tRNA binding sites (A, P and E), the peptidyl transferase center (PTC), and the peptide exit tunnel that extends through the body of the large subunit. The PTC is responsible for peptide bond formation and is located at the entrance to the peptide exit tunnel. As a result of peptide bond formation in the PTC, the nascent polymer chain is transferred from the peptidyl-tRNA in the P site to the aminoacyl-tRNA in the A site, thus extending the nascent chain by one amino acid. During translation, tRNAs translocate from the A to the P site and from the P to the E site.

As used herein, “translation” and “ribosome-directed translation” refers to the process whereby an RNA template (messenger RNA, or mRNA, or tmRNA) is converted by the action of ribosomes, with the help of tRNA and protein translation factors (TFs), into a polypeptide containing canonical and/or non-canonical amino acids, and is well known in the art. Translation involves an initiation step, whereby a ribosome attaches to the mRNA template, generally at the fMet codon (e.g., AUG) and initiator tRNA (fMet-tRNA^{fMet}) binds to the AUG codon displayed at the ribosomal P-site generally aided by initiation translation factors, followed by the elongation step, whereby the anti-codon of a charged tRNA molecule is paired with a selector codon in the RNA template, this step being facilitated by elongation TFs and repeated as the ribosome moves down the RNA template. As each tRNA anti-codon is paired with its corresponding selector codon, the amino group of the aminoacyl-tRNA molecule is covalently linked to the carboxyl group of the preceding amino acid via peptide bonds. In the case of α-hydroxyl-tRNA molecules, the carboxyl group of the preceding amino acid is linked via an ester bond. A tRNA moves sequentially from the A site, to the P site, and is finally translocated to the E site during each complete translational event that completes the formation of a peptide bond. Generally, translation also involves a termination step, whereby the ribosome encounters a translation stop codon, thus ending chain elongation and achieving the release of the polypeptide from the ribosome by action of protein release factors. However, in the methods described herein, the RNA template can comprise an ORF having a translational stop codon, which is recognized by the anti-codon of an acyl-tRNA analogue or in the absence of a release factor.

As used herein, “trans-translation” is used to describe a process which is performed on the ribosome by tmRNA in which aminoacyl-tmRNA, in complex with EF-Tu and SmpB, enters the A-site of ribosomes, stalled on an mRNA. The amino acid of the aminoacyl-tmRNA is transferred to the synthesized polypeptide; translation resumes on the tmRNA's ORF and terminates at its stop codon. The polypeptide elongated with the mRNA-like ORF coding sequence (MLR) is released.

As used herein, the term “genetically encoded” is used in a process whereby the information in at least one molecule is used in the production of a second molecule that has a different chemical nature from the first molecule. In reference to ribosome-directed translation an amino acid structure of a polypeptide, peptide, or protein is defined by an acyl-tRNA anti-codon interaction with a selector codon at a specific site on mRNA sequence. In reference to transcription, a DNA molecule can encode an RNA molecule (e.g., by a RNA polymerase enzyme), where transcription and/or translation may occur in a cell or in a cell-free in vitro transcription/translation system. Information in at least one molecule that is used to detect, but not direct, the production of a second molecule may be encoded, as e.g. barcoded DNA or RNA, if the encoded barcode remains physically or spatially linked to the encoded message that is translated.

As used herein, “substantially pure” means an object species is the predominant species present (i.e. on a molar basis it is more abundant than any other individual macromolecular species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. Solvent species, small molecules <1000 Daltons, and elemental ion species are not considered macromolecular species.

As used herein a “reactive moiety” or “reactive functional group” means a chemical group capable of undergoing a reaction with a second reactive moiety to yield a linker, and may be denoted as “x” or “y”. In general, the reactive moieties x and y are selected to form upon reaction a stable linker or spacer. However, in certain embodiments, it may be useful to choose the reactive functionalities x and y and reaction conditions so as to permit reversible reactions. In certain embodiments, x and y are the same. In other embodiments, x and y are different. In some embodiments, x and y are independently selected from the group consisting of thiols, protected thiols, disulfides, vinyl sulfones, epoxides, thiiranes, aziridines, esters, activated carboxylic acid derivatives, sulfonic acid esters, thioesters, carbonyls, 1,4-dicarbonyls, amines, azides, alkynes, alkenes, alcohols, phenols, aryl halides, boronate esters and imines, and the like. As will be appreciated by those of skill in the art, the reactive moieties may be chosen based on considerations of the speed of the reaction, solubility of reactants, reactant and catalyst concentrations, the absence of potential side products, reversibility of the reaction, etc. In some embodiments, the reaction of x and y occurs under conditions that would disrupt, denature, or degrade protein secondary and tertiary structures. For example, reactions may be carried out at low pH or in the presence of reducing agents.

As used herein, the term “orthogonally reactive” refers to the chemoselective or bio-orthogonal reactions of the mutually and uniquely reactive moieties x and y, which, while they occur in the presence of an RNA of interest, do not substantially chemically modify or alter the biological function of the RNA (including but not limited to tRNA) of interest, and/or the product of the orthogonal reaction is formed in high yield. The product may be substantially pure as well. For example, orthogonally reactive chemoselective reactions between x and y would take place but would modify less than 20% of the RNA of interest, preferably less than 10% of the RNA of interest, preferably less than 5% of the RNA of interest, preferably less than 1% of the RNA of interest, or even preferably less than 0.1% of the RNA of interest. In some embodiments chemoselective reactions between ligand reactive moieties L-x and tRNA-A-y may yield greater than 50% of the tRNA-A-z-L capable of ribosome-directed translation, greater than 60% of the tRNA-A-z-L capable of ribosome-directed translation, greater than 70% of the tRNA-A-z-L capable of ribosome-directed translation, preferably more than 80% of the tRNA-A-z-L capable of ribosome-directed translation, more preferably greater than 90% of tRNA-A-z-L capable of ribosome-directed translation, even more preferably greater than 95% of tRNA-A-z-L capable of ribosome-directed translation, or more than 99% of tRNA-A-z-L capable of ribosome-directed translation, or even more than 99.9% of tRNA-A-z-L capable of ribosome-directed translation. In some embodiments, the RNA of interest may be natural tRNA, unnatural tRNA and/or arbitrary tRNA. In some embodiments the RNA of interest may be acylated-tRNA. In some embodiments only the 3′ terminal ribonucleotides of tRNA are of interest.

As used herein the term “yield” or “percent yield” refers to a quantity formed of product of interest. Yield is calculated as percent conversion of the starting limiting reagent, where “conversion” is a measure of the quantity of starting limiting reagent material consumed to form a desired product. The term “high yield” as used herein refers to a useful molar percent yield in the range from about 60 percent to about 100 percent, and more preferably above about 80 percent. The “percent yield” may be determined by various analytical methods well known in the art, e.g. Ewing's Analytical instrumentation Handbook, 3rd ed. J, Cazes, CRC Press, 2005.

As used herein the term “ligand” or “ligand adduct” refers to a moiety that binds to, or has affinity for, a second molecule or receptor. As one of skill in the art will recognize, a molecule can be both a receptor and a ligand. Ligands are typically organic small molecules that have an intrinsic binding affinity for the target. Ligands may be catalytically active, participating in the making or breaking of chemical bonds, as in active-site residues of enzymes. Ligands may bind covalently to a second molecule or receptor. Ligand-receptor interactions are of interest for many reasons, from elucidating basic biological site recognition mechanisms to drug screening and rational drug design.

As used herein the term “bind” is used as a qualitative term to describe the strength of a ligand-target receptor interaction. A quantitative measure for the target binding affinity is expressed through the Association Constant (K_{A} in units of molarity). The Association Constant and the Dissociation Constant are related to each other by the equation K_{D}=1/K_{A}. Evidently, a high binding affinity corresponds to a lower Dissociation Constant. Binding may be defined in terms of the residence time of a receptor-ligand complex as described in Tummino, P. J. and Copeland, R. A., Biochemistry (2008) 47:5481-5492.

As used herein, “library” refers to a population of members that each occupy a unique three-dimensional space or are the same. A library can contain a few or a large number of different molecules, varying from about two to about 10¹⁵ molecules or more. The chemical structure of the molecules of a library can be related to each other or be diverse. The population members may be combined (pooled) or separated into different spatially addressable locations.

As used herein, the term “small molecules” or “small molecules” refers to molecules which are usually about 1,000 Da molecular weight or less, and include but are not limited to, synthetic organic or inorganic compounds, peptides, (poly)nucleotides, (oligo)saccharides and the like. Small molecules specifically include, inter alia, small non-polymeric (e.g., not peptide or polypeptide) organic and inorganic molecules. In one embodiment, small molecules have molecular weights of up to about 1,000 Da. In another embodiment small molecules have molecular weights of less than about 500 Da. In one embodiment, small molecules have molecular weights of up to about 250 Da. Included within this definition are small organic (including non-polymeric) molecules containing metals such as Zn, Hg, Fe, Cd, and As which may form a bond with nucleophiles.

In some embodiments of the methods provided herein, each member of the library of ligands with reactive moieties has a structure of the formula:

L-(CH₂)ₘ—X

where, m is an integer from 0 to 10, more preferably 0 to 2, and
wherein X is a moiety having one of the structures, but are not necessarily limited to: —O—NH₂ an amino-oxy or hydroxyamine group, —C(R)═O an aldehyde when R is hydrogen, and when —C(R)═O is a ketone, R is preferably C1-C10, more preferably C1 to C4, linear or branched alkyl group, —C≡C—H a terminal alkyne group, —N₃, an azide group, —C(R₁)═C(R₂)(R₃) a terminal alkene when R₂, R₃ are hydrogen, and when —C(R₁)═C(R₂)(R₃) is a substituted alkene, R₂, R₃ are independently hydrogen or preferably C1-C10, more preferably C1 to C4, linear or branched alkyl groups, —SH, a thiol, —NH₂, a primary amine, —SO₂—C(R₁)═C((R₂)(R₃), a vinyl sulfone group, —C(CH₂X′)═O, an alpha-halo methyl carbonyl group where X′ is a halogen, S—S—R₁, a disulfide group when R₁ is selected from H, alkyl, a carboxylic group and a heterocyclic group as described herein, and when —S—R₁ is a methanethiosulfonate group, R₁ is SO₂CH₃, and when —S—R₁ is a phenylthiosulfonate group, R₁ is —SO₂Ph, and when —S—R₁ is a phenylselenenyl, —S—R₁ is seleno aryl, —S—R₁ is a thiopyridyl group, —C═O(X″) an activated carboxyl ester, with X″ an activated leaving group, —Ar—X′, an aryl halide group where X′ is a halogen, —O—B(O—R₁)₂ a boronic acid diol ester wherein R₁ is an alkyl or cycloalkyl group, —C(R₁)═O(CH₂—CH₂)C(R₂)═O a beta-1,4-dicarbonyl group wherein each occurrence of R₁ and R₂ is independently hydrogen, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, or halo group, or an optionally substituted moiety —NO₂; —CN; —CF₃; —CH₂CF₃; —CHCl₂; —CH₂OH; —CH₂CH₂OH; —CH₂NH₂; —CH₂SO₂CH₃;— or -GR^{G1} wherein G is —O—, —S—.
and L is a moiety having one of the structures:

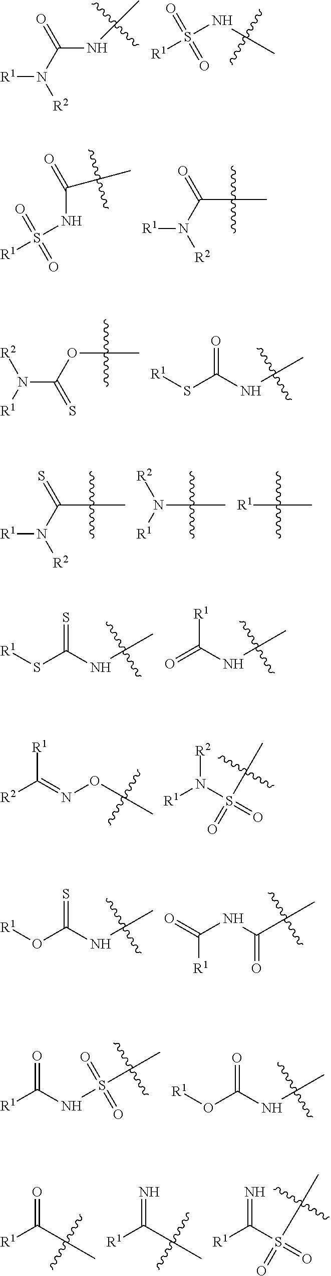

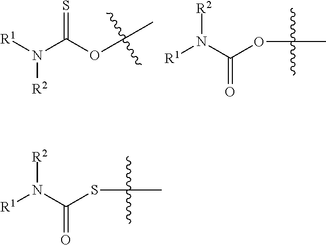

each occurrence of R¹ and R² is independently hydrogen, or aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, or wherein RAI and R^{A2} taken together are a cycloaliphatic, heterocycloaliphatic, aryl or heteroaryl moiety;
wherein each of the foregoing aliphatic and heteroaliphatic moieties is substituted or unsubstituted, linear or branched and each of the foregoing cycloaliphatic, heterocycloaliphatic, aryl or heteroaryl moieties is independently substituted or unsubstituted.

The term “substituted” or “optionally substituted” used herein in reference to a moiety or group means that one or more hydrogen atoms in the respective moiety, especially up to 5, more especially 1, 2 or 3 of the hydrogen atoms are replaced independently of each other by the corresponding number of the described substituents. The substituents may be the same or different and may be selected from hydroxy, halogen (e.g. fluorine), hydroxyalkyl (e.g. 2-hydroxyethyl), haloallcyl (e.g. trifluoromethyl or 2,2,2-trifluoroethyl), amino, substituted amino (e.g. N-alkyllamino, N,N-dialkylamino or N-alkanoylamino), alkoxycarbonyl, phenylalkoxycarbonyl, amidino, guanidino, hydroxyguanidino, formamidino, isothioureido, ureido, mercapto, acyl, acyloxy, carboxy, sulfo, sulfamoyl, carbamoyl, cyano, azo, nitro and the like.

A substituent is halogen or a moiety having from 1 to 30 plural valent atoms selected from C, N, O, S and Si as well as monovalent atoms selected from H and halo. In one class of compounds, the substituent, if present, is for example selected from halogen and moieties having 1, 2, 3, 4 or 5 plural valent atoms as well as monovalent atoms selected from hydrogen and halogen. The plural valent atoms may be, for example, selected from C, N, O, S and B, e.g. C, N, S and O. Examples of substituents include, but are not limited to aliphatic; heteroaliphatic; alicyclic; heteroalicyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃;— or -GR^{G1} wherein G is —O—, —S—, —NR^{G2}, —C(═O)—, —S(═O)—, —SO₂—, —C═O—, —C(═O)NR^{G2}, —OC(═O)—, —NR^{G2}C(═O)—, —OC(═O)O—, —OC(═O)NR.sup.G2-, —NR^{G2}C(═O)O—, —NR^{G2}(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR.sup.G2SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heteroalicyclic, aromatic, heteroaromatic, aryl, heteroaryl, alkylaryl, or alkylheteroaryl moiety.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognized by one skilled in the art.

As used herein, the term “aliphatic”, refers to and includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having about 1-6 carbon atoms.

As used herein, the term “alicyclic” refers to compounds which combine the properties of aliphatic and cyclic compounds and include, but are not limited to, cyclic, or polycyclic aliphatic hydrocarbons and bridged cycloalkyl compounds, which are optionally substituted with one or more functional groups. As will be appreciated by one of ordinary skill in the art, alicyclic is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norbornyl moieties and the like, which again, may bear one or more substituents.

As used herein, the term “cycloalkyl” refers specifically to cyclic alkyl groups having three to ten, preferably three to seven carbon atoms. Suitable cycloalkyls include, but are not limited to cyclopropyl, cyclobutyl, cyclopentyl, cyclohexyl, cycloheptyl and the like, which, as in the case of aliphatic, heteroaliphatic or heterocyclic moieties, may optionally be substituted. An analogous convention applies to other generic terms such as “cycloalkenyl”, “cycloalkynyl” and the like.

As used herein, the term “heteroaliphatic” refers to aliphatic moieties in which one or more carbon atoms in the main chain have been substituted with a heteroatom. Thus, a heteroaliphatic group refers to an aliphatic chain which contains, among other possibilities, one or more oxygen, sulfur, nitrogen, phosphorus or silicon atoms, i.e., in place of carbon atoms. Thus, a 1-6 atom heteroaliphatic linker having at least one N atom in the heteroaliphatic main chain, as used herein, refers to a C₁₋₆aliphatic chain, wherein at least one carbon atom is replaced with a nitrogen atom, and wherein any one or more of the remaining 5 carbon atoms may be replaced by an oxygen, sulfur, nitrogen, phosphorus or silicon atom. As used herein, a 1-atom heteroaliphatic linker having at least one N atom in the heteroaliphatic main chain refers to —NH— or —NR— where R is aliphatic, heteroaliphatic, acyl, aromatic, heteroaromatic or a nitrogen protecting group. Heteroaliphatic moieties may be branched or linear unbranched. In certain embodiments, heteroaliphatic moieties are substituted by independent replacement of one or more of the hydrogen atoms thereon with one or more moieties including, any of the substituents described above.

As used herein, the term “heteroalicyclic”, “heterocycloalkyl” or “heterocyclic” refers to compounds which combine the properties of heteroaliphatic and cyclic compounds and include, but are not limited to, saturated and unsaturated mono- or polycyclic heterocycles such as morpholino, pyrrolidinyl, furanyl, thiofuranyl, pyrrolyl, etc., which are optionally substituted with one or more functional groups, as defined herein. In certain embodiments, the term “heterocyclic” refers to a non-aromatic 5-, 6- or 7-membered ring or a polycyclic group, including, but not limited to, a bi- or tri-cyclic group comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds, and each 7-membered ring has 0 to 3 double bonds (ii) the nitrogen and sulfur heteroatoms may optionally be oxidized, (iii) the nitrogen heteroatom may optionally be quaternized, and (iv) any of the above heterocyclic rings may be fused to an aryl or heteroaryl ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

Additionally, it will be appreciated that any of the alicyclic or heteroalicyclic moieties described above and herein may comprise an aryl or heteroaryl moiety fused thereto. Additional examples of generally applicable substituents are illustrated by the specific embodiments shown in the Examples that are described herein.

As used herein, the term “aromatic moiety” refers to stable substituted or unsubstituted unsaturated mono- or poly-cyclic hydrocarbon moieties having preferably 3-14 carbon atoms, comprising at least one ring satisfying the Huckel rule for aromaticity. Examples of aromatic moieties include, but are not limited to, phenyl, indanyl, indenyl, naphthyl, phenanthryl, and anthracyl.

As used herein, the term “heteroaromatic moiety” refers to stable substituted or unsubstituted unsaturated mono-heterocyclic or polyheterocyclic moieties having preferably 3-14 carbon atoms, comprising at least one ring satisfying the Huckel rule for aromaticity. Examples of heteroaromatic moieties include, but are not limited to, pyridyl, quinolinyl, dihydroquinolinyl, isoquinolinyl, quinazolinyl, dihydroquinazolyl, and tetrahydroquinazolyl.

It will also be appreciated that aromatic and heteroaromatic moieties, as defined herein, may be attached via an aliphatic (e.g., alkyl) or heteroaliphatic (e.g., heteroalkyl) moiety to provide moieties such as -(aliphatic)aromatic, -(heteroaliphatic)aromatic, -(aliphatic)heteroaromatic, -(heteroaliphatic)heteroaromatic, -(alkyl) aromatic, -(heteroalkyl)aromatic, -(alkyl)heteroaromatic, and -(heteroalkyl)heteroaromatic moieties. Substituents of these moieties include, but are not limited to, any of the previously mentioned substituents resulting in the formation of a stable compound.

As used herein, the term “aryl” refers to aromatic moieties, as described above. In certain embodiments of the present invention, “aryl” refers to a mono- or bicyclic carbocyclic ring system having one or two rings satisfying the Huckel rule for aromaticity, including, but not limited to, phenyl, naphthyl, tetrahydronaphthyl, indanyl, indenyl and the like.

As used herein, the term “heteroaryl” refers to heteroaromatic moieties, as described above, without limitation. In certain embodiments of the present invention, the term heteroaryl, as used herein, refers to a cyclic unsaturated radical having from about five to about ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

Substituents for aryl and heteroaryl moieties include, but are not limited to, any of the previously mentioned substitutents, i.e., the substituents recited for aliphatic moieties, or for other moieties as disclosed herein, resulting in the formation of a stable compound.

As used herein, the terms “alkoxy” (or “alkyloxy”), and “thioalkyl” refer to an alkyl group, as previously defined, attached to the parent molecular moiety through an oxygen atom (“alkoxy”) or through a sulfur atom (“thioalkyl”), respectively. In certain embodiments, the alkyl group contains about 1-20 aliphatic carbon atoms. In certain other embodiments, the alkyl group contains about 1-10 aliphatic carbon atoms. In yet other embodiments, the alkyl group contains about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkoxy groups, include but are not limited to, methoxy, ethoxy, propoxy, isopropoxy, n-butoxy, tert-butoxy, neopentoxy and n-hexoxy. Examples of thioalkyl groups include, but are not limited to, methylthio, ethylthio, propylthio, isopropylthio, n-butylthio, and the like.

As used herein, the term “amine” refers to a group having the structure —N(R)₂ wherein each occurrence of R is independently hydrogen, or an aliphatic, heteroaliphatic, aromatic, heteroaromatic, -(alkyl)aromatic, -(heteroalkyl)aromatic, (heteroalkyl)heteroaromatic, or -(heteroalkyl)heteroaromatic moiety, or the R groups, taken together with the nitrogen to which they are attached, may form a heterocyclic moiety.

As used herein, the term “aminoalkyl” refers to a group having the structure NH₂R′—, wherein R′ is alkyl, as defined herein. In certain embodiments, the alkyl group contains about 1-20 aliphatic carbon atoms. In certain other embodiments, the alkyl group contains about 1-10 aliphatic carbon atoms. In yet other embodiments, the alkyl, alkenyl, and alkynyl groups employed in the invention contain about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkylamino include, but are not limited to, methylamino, ethylamino, isopropylamino and the like.

As used herein, the terms “halo” and “halogen” as used herein refer to an atom selected from fluorine, chlorine, bromine and iodine.

As used herein, the term “halogenated” denotes a moiety having one, two, or three halogen atoms attached thereto.

As used herein, the term “haloalkyl” denotes an alkyl group, as defined above, having one, two, or three halogen atoms attached thereto and is exemplified by such groups as chloromethyl, bromoethyl, trifluoromethyl, and the like.

As used herein, the term “acyloxy” does not substantially differ from the common meaning of this term in the art, and refers to a moiety of structure —OC(O)Rₓ, wherein Rₓ is a substituted or unsubstituted aliphatic, alicyclic, heteroaliphatic, heteroalicyclic, aryl or heteroaryl moiety.

As used herein, the term “acyl” does not substantially differ from the common meaning of this term in the art, and refers to a moiety of structure —C(═O)ORₓ wherein Rₓ is a substituted or unsubstituted, aliphatic, alicyclic, heteroaliphatic, heteroalicyclic, aryl, or heteroaryl moiety.

As used herein, the term “imino” does not substantially differ from the common meaning of this term in the art, and refers to a moiety of structure —C(═NRₓ)R_{y}, wherein Rₓ is hydrogen or an optionally substituted aliphatic, alicyclic, heteroaliphatic, heteroalicyclic, aryl or heteroaryl moiety; and R_{y} is an optionally substituted aliphatic, alicyclic, heteroaliphatic, heteroalicyclic, aryl or heteroaryl moiety.

As used herein, the term “C₁₋₆alkenylene” refers to a substituted or unsubstituted, linear or branched saturated divalent radical consisting solely of carbon and hydrogen atoms, having from one to six carbon atoms, having a free valence “-” at both ends of the radical.

As used herein the term “C₂₋₆alkenylene” refers to a substituted or unsubstituted, linear or branched unsaturated divalent radical consisting solely of carbon and hydrogen atoms, having from two to six carbon atoms, having a free valence “-” at both ends of the radical, and wherein the unsaturation is present only as double bonds and wherein a double bond can exist between the first carbon of the chain and the rest of the molecule.

As used herein, the terms “aliphatic”, “hetero aliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aromatic”, “hetero aromatic”, “aryl”, “heteroaryl”, and the like, used alone or as part of a larger moiety, encompass both substituted and unsubstituted groups.

As used herein, the term “protecting group,” refers to a labile chemical moiety which is known in the art to protect an functional group against undesired reactions during synthetic procedures. After said synthetic procedure(s) the protecting group as described herein may be selectively removed. Protecting groups as known in the art are described generally in Protective Groups in Organic Synthesis (Third Edition) Greene, T. W. and Wuts, P. G. M., (2002). Examples of aldehyde protecting groups include, but are not limited to, vinyl ethers, and the like.

As used herein, the terms “link”, “linked”, “linkage” and variants thereof comprise any type of fusion, bond, ligation, adherence or association that is of sufficient stability to withstand use in the particular biological or chemical application of interest. Such linkage can comprise, for example, covalent bonds, ionic bonds, or hydrogen bonds, ligations or bonds between two entities, dipole-dipole interactions, hydrophilic, hydrophobic, or affinity bonding, bonds or associations involving van der Waals forces, mechanical bonding, and the like. Some examples of linkages can be found, for example, in Hermanson, G., Bioconjugate Techniques, Second Edition (2008); Aslam, M., Dent, A., Bioconjugation: Protein Coupling Techniques for the Biomedical Sciences, London: Macmillan (1998); Phelps, K., et al., ACS Chem Biol (2012) 7:100-109.

As used herein, the term “linker” and its variants comprises any composition, including any molecular complex or molecular assembly, that serves to link two or more compounds. Optionally, such linkage can occur between a combination of different molecules, including but not limited to: between an mRNA and a tRNA; between an mRNA, a tRNA, and a ribosome; between an mRNA and modified DNA and a polypeptide; between a cDNA and RNA, and the like. As will be obvious to those of skill in the art, such linkage can vary in time, space, and strength of interaction, depending on various conditions known in the art.

As used herein, “target” or “target protein” refers to any kind of protein or polypeptide amenable to influence by the binding of another molecule. In the case of protein targets a list of applicable targets may be obtained e.g. by accessing a public database such as a NCBI database (http://www.ncbi.nlm.nih.gov/entrez/guery.fcgi?db=Protein). In the case of human enzymes and receptors, targets may be retrieved from said database using e.g. “Human” and “Enzyme” or “Receptor” as query keywords. Moreover, a list of targets can be retrieved from the “Mode of Action” section of the Medtrack database (medtrack.com). Typical categories of targets include, but are not limited to enzymes, cytokines, receptors, transporters and channels. In the present invention compounds are identified which modulate the activity of the target protein in a desirable way, for example modulation of the protein's biological activity, such as inhibition of the activity of the target protein or stimulation of the activity of the target protein. In one embodiment, the target represents a druggable molecule, that is, molecules which allow the development of lead structures or drugs interacting therewith in order to inhibit biological function, activate biological function, or target expression thereof. In particular, these targets are drug targets for drugs of the group of small molecules or peptidomimetics.

In one embodiment, the target is a polypeptide, especially a protein. Polypeptides, including proteins, that find use herein as targets for binding ligands, such as, for example small organic molecule ligands, include virtually any polypeptide (including short polypeptides also referred to as peptides) or protein that comprises two or more binding sites of interest. Polypeptide targets of interest may be obtained commercially, recombinantly, by chemical synthesis, by purification from natural source, or other approaches known to those of skill in the art.

In one embodiment the target is a protein associated with a specific human disease or condition. Therapeutic drug targets can be divided into different classes according to function; receptors, enzymes, hormones, transcription factors, ion channels, nuclear receptors, DNA, (Drews, J. (2000) Science 287:1960-1964). Such targets include cell surface and soluble receptor proteins, such as lymphocyte cell surface receptors, G-protein coupled receptors (GPCRs), melanocortin receptors, cannabinoid receptors, free fatty acid receptors, enzymes, steroid receptors, nuclear proteins, allosteric enzymes, clotting factors, bacterial enzymes, fungal enzymes and viral enzymes (especially those associated with HIV, influenza, rhinovirus and RSV), signal transduction molecules, transcription factors, proteins or enzymes associated with DNA and/or RNA synthesis or degradation, immunoglobulins, hormones, various chemokines and their receptors, various ligands and receptors for tyrosine kinases, various neurotrophins and their ligands, other hormones and receptors and proteins, and immune-checkpoint proteins, such as programmed cell death protein 1 (PD1) and its receptor CD47.

In another variation, the target is selected from the group of human inflammation and immunology targets including IgE/IgER, ZAP-70, lck, syk, ITK/BTK, TACE, Cathepsins S and F, CD11a, LFA/ICAM, VLA-4, CD28/B7, CTLA-4, TNF alpha and beta, (and the p55 and p75 TNF receptors), CD40L, p38 map kinase, IL-2, IL-4, 11-13, IL-15, IL-17a, IL-19, IL-23, Rac 2, PKC theta, TAK-1, jnk, IKK2, IL-18, Jak2, Jak3, C3, C5a, C5, Factor D.

In another variation, the target is selected from the group of human metabolic disease targets consisting of PPAR, GLP-1 receptor, DPP4, PTP-1B, 5HT2c.

In another variation, the target is selected from the group of oncology targets including EGFR, TNFalpha, CD11a, CSFR, CTLA-4, KIR receptor, NKG2D receptor, MICA, SIRPa, EpCAM, VEGF, CD40, CD20, CD30, CD47, Notch 1, Notch 2, Notch 3, Notch 4, Jagged 1, Jagged 2, Frizzled-7, p53, BCL CD52, MUC1, IGF1R, transferrin, gp130, VCAM-1, CD44, DLL4, IL4, cMet, HGF, PSMA, Anti-Lewis-Y, Collagen, hGH, IL4R, RAAG12 Apelin J receptor, Hyaluronidase, IL6, Sphingosine 1 Phosphate, TIM3, SMO receptor, receptor tyrosine kinases such as members of the platelet-derived growth factor receptor (PDGFR), vascular endothelial growth factor receptor (VEGFR) families, and intracellular proteins such as members of the Syk, SRC, and Tec families of kinases, Burton's tyrosine kinase (BTK), PI3 kinase, Pim-1 kinase, Interleukin-2 inducible T-cell kinase (ITK), ERK2, MAPK, Akt-2, MEKK-1, CDK2, CDK4, Aurora kinases, B-raf, FMS kinase, KIT kinase, immune activating T-cell receptors such as CD28, OX40, GITR, CD137, CD27, HVEM, T-cell inhibitory receptors CTLA-4, PD-1, TIM-3, BTLA, VISTA, LAG-3, and tumor cell receptors such as ICOS, PD-L1, B7-H3, B7-H4.

In another variation, the target is selected from the group of undruggable proteins or protein-protein interactions such as caspases 1, 3, 8, and 9, IL-1/IL-1 receptor, BACE1, kallikrein, HIV integrase, PDE IV, Hepatitis C helicase, Hepatitis C protease, rhinovirus protease, tryptase, cPLA (cytosolic Phospholipase A2), CDK4, c-jun kinase, adaptors such as Grb2, GSK-3, PAK-1, raf, TRAFs 1-6, Tie2, ErbB 1 and 2, FGF, PDGF, PARP, CD2, C5a receptor, CD4, CD26, CD3, TGF-alpha, NF-κB, IKK beta, STAT 6, Neurokinin-1 receptor, PTP-1B, CD45, Cdc25A, SHIP-2, TC-PTP, PTP-alpha, LAR, p53, mdm2, HSP90.

In another embodiment, the target protein is a protein that is involved in apoptosis. For example, the target may be a member of the Bcl-2 (Bcl lymphoma 2) family of proteins, which are involved in mitochondrial outer membrane permeabilization. The family includes the proapoptotic proteins Bcl-2, Bcl-X_{L}, Mc1-1, CED-9, Al, and Bfl-1; and includes the antiapoptotic proteins Bax, Bak, Diva, Bcl-XS, Bik, Bim, Bad, Bid, and Egl-1.

In another embodiment, the target protein is a protein that is involved in epigenetic regulation such as lysine specific demethylase 1 (LSD1). The target may be a member of the BET family of bromodomain containing proteins which contain tandem bromodomains capable of binding to two acetylated lysine residues. The family includes the proteins BRD2, BRD3, BRD4 and BRD-T.

In another embodiment, the target protein is an ion channel selected from the group comprising a potassium ion channel, sodium ion channel, or acid sensing ion channel. In some embodiments the channel may be voltage-gated. The ion channel may be selected from the group comprising Kv1.3 ion channel, Nav1.7 ion channel, and acid sensing ion channel (ASIC).

As used herein, “spatially addressable” is used to describe how different molecules may be identified on the basis of their position on an array, see, for example, He, M., et al., Nat Methods (2008) 5:175-177.

As used herein, the term “isolating” or “isolating polypeptide products of translation” means isolating a translated protein/target complex formed in a reaction mixture. Such methods involve combining a translated polypeptide with target molecule under conditions that allow polypeptides specific for the target molecule to associate to form a translated protein/target complex. Typically, a pool containing a plurality of different translated species is combined in a reaction mixture containing the target molecule. If a translated species specific for the target molecule is present in the pool, translated protein/target complexes are formed. Such complexes can then be isolated from the other reaction components by methods well known in the art. Such methods include, but are not limited to, affinity purification, selection by catalysis, fluorescence sorting, in vivo selections, cell-based selections, and the like. As used herein, an “isolating” step provides at least a 2-fold, preferably, a 30-fold, more preferably, a 100-fold, and, most preferably, a 1000-fold enrichment of a desired molecule relative to undesired molecules in a population following the isolation step. As indicated herein, an isolation step may be repeated any number of times, and different types of isolation steps may be combined in a given approach.

As used herein, the term “identifying” or “identifying polypeptide products of translation” means determining at least the sequence of amino acids and non-canonical amino acid ligand adducts comprising the polypeptide. Information regarding the polypeptide sequence may be determined by reverse transcription-PCR of its associated mRNA or RNA barcodes, if the polypeptide products of translation remain physically linked to the message that encodes it, (or linked long enough so that the absence, presence, or quantity of, for example, the polyeptide products correlates with the absence, presence, or quantity of the message which encodes it), followed by DNA sequencing. Information regarding a polypeptides molecular weight, three-dimensional structure, etc. may also be determined, if desired, using any suitable technique, e.g. mass spectrometry, solution NMR, and powder and single crystal diffraction.

The limited chemical and shape diversity, as well as the biological instability of natural polypeptides selected from biological libraries can make the generation of drug-like compounds very challenging. The present invention provides compositions and methods for generating diverse chemical structures capable of ribosome-directed translation into polymeric structures with drug-like properties.

The invention was developed because of the need for methods to rapidly and simply encode, select, and decode diverse chemical structures in biopolymer backbones, despite considerable effort over many years by many workers skilled in the art. The chemical diversity provided by the methods of the invention is unprecedented in ribosome-directed libraries that have heretofore appeared to have a low probability of success.

The encoded chemical diversity of the invention provides several advantages over other screening technology approaches. For example, the method of the invention enables a rapid survey of relevant chemical space for target binding through genetically encoded chemical ligands. The method of the invention is applicable to development of ligands for a variety of targets, including large proteins, protein complexes, and partially purified fractions. Another advantage of the method of the invention is that the three-dimensional structure of the target need not be characterized. Another advantage is that the method provides high sensitivity with low protein consumption. Relatedly, the method enables use commercial protein sources, which can provide substantial savings in time and cost over methods that require manufacture of variants, truncates, and modified versions of targets of interest. Another advantage is that binding is not subject to artifacts due to solubility, since library/target complexes can be incubated at low concentrations. Another advantage is that direct ligand competitors can be used to tune the residence time of a receptor-ligand complex. Another advantage of the present invention is that it can be configured to provide a direct readout of inhibition of the activity of a protein or other target by the candidate ligand adduct. Another advantage of the method is that the spatial relationship between two ligand adduct moieties may the decoded from the polypeptide sequence, based on known rules of protein folding and secondary structure. In contrast, current methods of fragment-based drug discovery required complex methods for efficient optimization of fragment hits into lead structures, cf. Erlanson, D. A., Top Curr Chem (2012) 317:1-32.

Yet another advantage of the invention is that the ligand adducts are assembled prior to translation, so that the translated chemical sequence and structure is exactly known, and not contaminated with unliganded amino acid side chains. This is in contrast to posttranslationally modified ligands, where fragments are assembled after translation and, therefore, must occur under conditions that preserve polypeptide scaffold integrity and purity, see for example Frankel, A., et al., Curr Opin Struct Biol (2003) 13:506-512. Conducting fragment assembly under pre-translational conditions according to the invention allows the use of a multitude of different chemistries and presents new opportunities for efficiently introducing chemical diversity into multiple different scaffold proteins. It offers the ability to use a single library of acyl-tRNA ligand adducts with multiple mRNA sequences of interest. It also offers a strategy for combining binding kinetics with binding site analysis of structure activity relationships-something that is very inefficiently and inadequately addressed by current methods.

Yet another advantage of the invention is that ligand adduct libraries may be evolved for function. Large (10¹² member) ligand adduct libraries displayed on protein or peptide scaffolds that have affinity for a desired target protein may be selected for by sequential rounds of binding, enrichment, amplification, sequencing, library redesign, and translation. Other methods of encoded chemical libraries require very efficient methods of chemical screening in a single round with high false positive hit rates, without the ability to evolve the libraries for function.

### General Methods

Unless defined otherwise, all technical and scientific terms used herein have the meaning commonly understood by one of ordinary skill in the art to which this invention belongs. Practitioners are particularly directed to Green, M. R., and Sambrook, J., eds, Molecular Cloning: A Laboratory Manual, 4th ed., Cold Spring Harbor Laboratory Press, Cold Spring Harbor, N.Y. (2012), and Ausubel, F. M., et al., Current Protocols in Molecular Biology (Supplement 99), John Wiley & Sons, New York (2012), Bornscheuer, U. and Kazlauskas, R. J., Curr Protoc Protein Sci (2011) Chapter 26:Unit26 27 which describes methods of protein engineering, which are incorporated herein by reference, for definitions and terms of the art. Standard methods also appear in Bindereif, Scholl, & Westhof (2005) Handbook of RNA Biochemistry, Wiley-VCH, Weinheim, Germany which describes detailed methods for RNA manipulation and analysis, and Beaucage, S. L. and Reese, C. B., Curr Protoc Nucleic Acid Chem (2009) Chapter 2:Unit 2 16 11-31; Keel, A. Y., et al., Methods Enzymol (2009) 469:3-25 which describe methods of chemical synthesis and purification of RNA, and are incorporated herein by reference. Examples of appropriate molecular techniques for generating nucleic acids, and instructions sufficient to direct persons of skill through many cloning exercises are found in Green, M. R., and Sambrook, J., (Id.); Ausubel, F. M., et al., (Id.); Berger and Kimmel, Guide to Molecular Cloning Techniques, Methods in Enzymology (Volume 152 Academic Press, Inc., San Diego, Calif. 1987); and PCR Protocols: A Guide to Methods and Applications (Academic Press, San Diego, Calif., 1990), which are incorporated by reference herein.

Methods for protein purification, chromatography, electrophoresis, centrifugation, and crystallization are described in Coligan et al. (2000) Current Protocols in Protein Science, Vol. 1, John Wiley and Sons, Inc., New York. Methods for cell-free protein synthesis are described in Endo, Y., et al., (2010). Methods for incorporation of non-natural amino acids into polypeptides using cell-free protein synthesis are described in Smolskaya, S., et al., PLoS ONE (2013) 8:e68363 and references cited therein.

PCR amplification methods are well known in the art and are described, for example, in Innis et al., PCR Protocols: A Guide to Methods and Applications, Academic Press Inc. San Diego, Calif., 1990. An amplification reaction typically includes the DNA that is to be amplified, a thermostable DNA polymerase, two oligonucleotide primers, deoxynucleotide triphosphates (dNTPs), reaction buffer and magnesium. Typically a desirable number of thermal cycles is between 1 and 25. Methods for primer design and optimization of PCR conditions are well known in the art and can be found in standard molecular biology texts such as Ausubel et al., Short Protocols in Molecular Biology, 5^{th} Edition, Wiley, 2002, and Innis et al., PCR Protocols, Academic Press, 1990. Computer programs are useful in the design of primers with the required specificity and optimal amplification properties (e.g., Oligo Version 5.0 (National Biosciences) or PrimerQuest (www.idtdna.com)). In some embodiments, PCR primers may additionally contain recognition sites for restriction endonucleases, to facilitate insertion of the amplified DNA fragment into specific restriction enzyme sites in a vector. If restriction sites are to be added to the 5′ end of PCR primers, it is preferable to include a few (e.g., two or three) extra 5′ bases to allow more efficient cleavage by the enzyme. In some embodiments, PCR primers may also contain an RNA polymerase promoter site, such as T7 or SP6, to allow for subsequent in vitro transcription. Methods for in vitro transcription are well known to those of skill in the art (see, e.g., Van Gelder et al., Proc. Natl. Acad. Sci. U.S.A. (1990), 87:1663-1667; Eberwine et al., Proc. Natl. Acad. Sci. U.S.A. (1992), 89:3010-3014).

Reverse transcription (RT) may be used to prepare template DNA from an initial RNA sample, e.g. mRNA, which template DNA is then amplified using PCR to produce a sufficient amount of amplified product for the application of interest such as DNA sequencing. The RT and PCR steps of DNA amplification can be carried out as a two-step or one step process. In an effort to further expedite and simplify RT-PCR procedures, a variety of RT-PCR and related quantitative real-time RT-PCR (qRT-PCR) protocols have been developed, see: Freeman, W. M., et al., Biotechniques (1999) 26:112-122, 124-115. For example, errors introduced by reverse transcriptase enzymes can be minimized using commercial high-fidelity retroviral reverse transcriptases or thermostable group II intron reverse transcriptases (Mohr, S., et al., RNA (2013) 19:958-970).

The aminoacyl-tRNAs as well as polypeptides synthesized by ribosome-directed translation can be separated from a reaction mixture and further purified by methods such as column chromatography, including affinity-based, charged-based, and other chromatography methods, fast protein liquid chromatography, high pressure liquid chromatography, capillary electrophoresis, precipitation, and extraction. As can be appreciated by the skilled artisan, further methods of synthesizing the small-molecule ligand reactive moieties contemplated herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 25 2nd Ed. Wiley-VCR (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Amino Acids with Orthogonally Reactive Moieties

As described above, in one preferred use, the amino acid comprises an orthogonally reactive moiety. The orthogonally reactive moiety can be any functional group known to those of skill in the art. In certain embodiments the moiety is an orthogonally reactive functional group. Orthogonally reactive functional groups are particularly advantageous for chemoselective ligations of further moieties attached to the amino acid side chain on an acylated tRNA that genetically encodes the 3′ acylated amino acid at selector codon of an mRNA sequence during ribosome-directed translation.

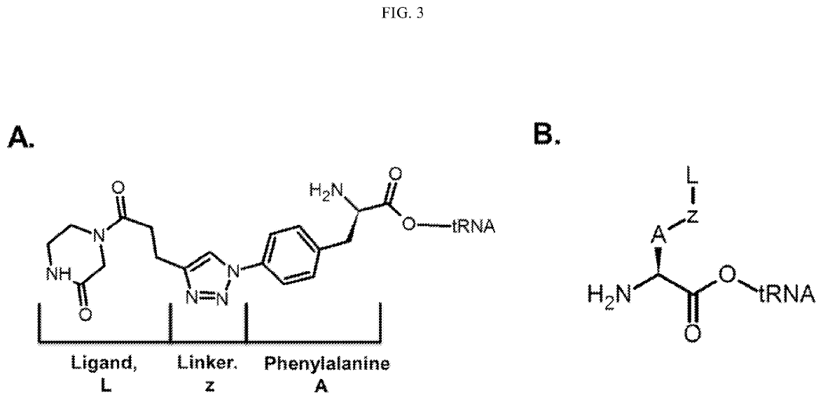

In some embodiments, the non-canonical amino acids include side chain functional groups or moieties that react efficiently and chemoselectively with functional groups, or moieties not found in ribonucleic acids (including but not limited to azido, alkynl, alkenyl, aryl halide, alkyl halide, boronate, activated carbonyl esters, 1,4 dicarbonyl, aldehyde, and aminooxy groups) to form stable linkers. In certain embodiments, the reactive moiety is selected from the group consisting of amino, aminoxy, azido, alkynyl, thiol, phospho, or hydroxyl moieties. For example, an aminoacyl-tRNA with a non-canonical amino acid containing an azide moiety can form a stable linker resulting from the selective reaction of the azide and a terminal alkyne functional group to form a Huisgen [3+2] cycloaddition product, as illustrated in FIG. 3. In certain embodiments reactive moieties that may react slowly with ribonucleic acids including phosphodiester bonds or cytosine carbonyls, under certain conditions may still react orthogonally under other conditions known in the art.

In further embodiments, non-canonical amino acids with side chains containing reactive moieties that may be used in the methods and compositions described herein include, but are not limited to, amino acids with novel functional groups, amino acids that covalently or noncovalently interact with other molecules, but not with ribonucleic acids, photocaged and/or photoisomerizable amino acids, chemically cleavable and/or photocleavable amino acids, amino acids comprising biotin or a biotin analogue, aldehyde-containing amino acids, and redox-active amino acids, and amino acids with side chains modifiable by enzymes or catalytic antibodies.

The non-canonical amino acids used in the present invention typically comprise one or more modified derivatives or analogues of amino acids, wherein the chemical structures have the formula NH₂—(HCR)—COOH, where R is not any of the 20 canonical substituents defining the canonical amino acids. Suitable non-canonical amino acid derivatives are commercially available from vendors such as, e.g., Bachem Inc., (Torrance, Calif.); Genzyme Pharmaceuticals (Cambridge, Mass.); Asiba Pharmaceuticals (Trenton, N.J.); Chem-Impex International, Inc. (Wood Dale, Ill.); Sigma-Aldrich (St. Louis, Mo.); Synthetec, Inc. (Albany, Oreg.). Preferably, the non-canonical amino acids include but are not limited to derivatives and/or analogs of glycine, tyrosine, glutamine, phenyalanine, serine, threonine, proline, tryptophan, leucine, methionine, lysine, alanine, arginine, asparagine, valine, isoleucine, aspartic acid, glutamic acid, cysteine, histidine, as well as beta-amino acids and homologs, BOC-protected amino acids, and FMOC-protected amino acids, N-alkyl amino acids, α,α-disubstituted amino acids, and D-amino acids.

The generation of non-canonical amino acid derivatives, analogs, and mimetics not already commercially available can be accomplished in several ways. For example, one way is to synthesize a non-canonical amino acid of interest using organic chemistry methods known in the art, while another way is to utilize chemoenzymatic synthesis methods known in the art. See, e.g., Kamphuis et al., Ann. N. Y. Acad. Sci., 672:510-527, 1992; Ager D J and Fotheringham I G, Curr. Opin. Drug Discov. Devel., 4:800-807, 2001; Weiner et al., Chem. Soc. Rev., 39:1656-1691, 2010; Asymmetric Syntheses of Unnatural Amino Acids and Hydroxyethylene Peptide Isosteres, Wieslaw M. Kazmierski, ed., Peptidomimetics Protocols, Vol. 23, 1998; and Unnatural Amino Acids, Kumar G. Gadamasetti and Tamim Braish, ed., Process Chemistry in the Pharmaceutical Industry, Vol. 2, 2008; Wang L et al., Chemistry and Biology, 16:323-336, 2009; and Wang F, Robbins S, Guo J, Shen W and Schultz P G., PLoS One, 5:e9354, 2010. One skilled in the art will recognize that many procedures and protocols are available for the synthesis of non-canonical amino acids.

The non-canonical amino acids may include L- and D-alpha amino acids. L-alpha amino acids can be chemically synthesized by methods known in the art such as, but not limited to, hydrogen-mediated reductive coupling via rhodium-catalyzed C—C bond formation of hydrogenated conjugations of alkynes with ethyl iminoacetates (Kong et al., J. Am. Chem. Soc., 127:11269-11276, 2005). Alternatively, semisynthetic production by metabolic engineering can be utilized. For example, fermentation procedures can be used to synthesize non-native amino acids from E. coli harboring a re-engineered cysteine biosynthetic pathway, see Maier T H, Nature, (2003), 21:422-427). Racemic mixtures of alpha-amino acids can be produced using asymmetric Strecker syntheses (as described in Zuend et al., Nature, (2009) 461:968-970) or using transaminase enzymes for large-scale synthesis (as found in Taylor et al., Trends Biotechnol., (1998).16:412-419. Bicyclic tertiary alpha-amino acids may be produced by alkylation of glycine-derived Schiff bases or nitroacetates with cyclic ether electrophiles, followed by acid-induced ring opening and cyclization in NH₄OH (see Strachan et al., J. Org. Chem., (2006) 71:9909-9911).

The non-canonical amino acids may further comprise beta-amino acids, which are remarkably stable to metabolism, exhibit slow microbial degradation, and are inherently stable to proteases and peptidases. An example of the synthesis of beta amino acids is described in Tan, C Y K and Weaver, D F, Tetrahedron, (2002) 58:7449-7461.

In some instances, the non-canonical amino acids comprise chemically modified amino acids commonly used in solid phase peptide synthesis, including but not limited to, tert-butoxycarbonyl- (Boc) or (9H-fluoren-9-ylmethoxy)carbonyl (Fmoc)-protected amino acids. For example, Boc derivatives of leucine, methionine, threonine, tryptophan and proline can be produced by selective 3,3-dimethyldioxirane side-chain oxidation, as described in Saladino et al., J. Org. Chem., (1999) 64:8468-8474. Fmoc derivatives of alpha-amino acids can be synthesized by alkylation of ethyl nitroacetate and transformation into derivatives (see Fu et al., J. Org Chem., (2001) 66:7118-7124).

### Acylated TRNA

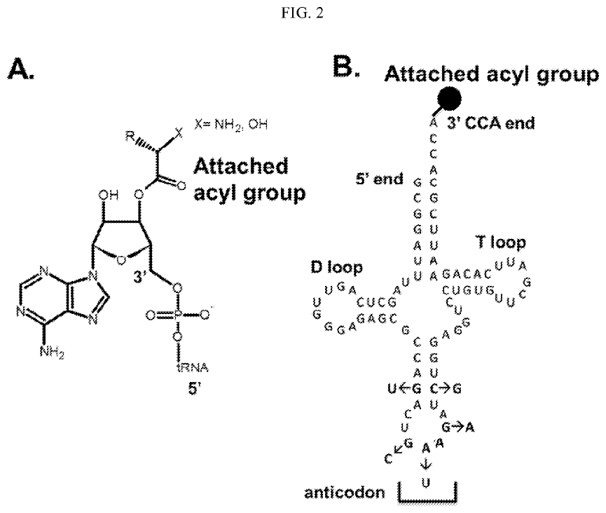

In order to genetically encode the ligand adduct moieties linked to canonical amino acids, α-hydroxyl acids, and non-canonical amino acids into a desired polymer, canonical amino acids, α-hydroxyl acids, and non-canonical amino acids are linked to the 3′ hydroxyl of selector codon reading tRNAs via an acyl ester to form acylated tRNA (FIG. 2), for subsequent formation of acylated tRNAs with non-canonical amino acid ligand adducts (FIG. 3). The tRNA acylation reaction, as used herein, refers to the in vitro tRNA acylation reaction in which desired selector codon reading tRNAs are acylated with their respective canonical amino acids, or α-hydroxyl acid, or non-canonical amino acid of interest. The tRNA acylation reaction comprises the acylation reaction mixture, a selector codon reading tRNA, and as used in this invention, may include either canonical amino acids or α-hydroxyl acids, non-canonical amino acids or α-hydroxyl acids with an orthogonally reactive moiety. The tRNA acylation reaction can occur in a separate reaction, where the charged tRNA is then added to the cell-free translation reaction. Alternatively, the tRNA acylation reaction occurs in the presence of a cell-free translation system.

Methods for modifying tRNA including, but not limited to, modifying the anti-codon, the amino acid attachment site, and/or the accepter stem to allow incorporation of unnatural and/or arbitrary amino acids are known in the art (Xie, J. and Schultz, P. G., Methods (2005) 36:227-238; Sisido, M., et al., Methods (2005) 36:270-278; Wang, L., et al., Annu Rev Biophys Biomol Struct (2006) 35:225-249; Liu, C. C. and Schultz, P. G., Annu Rev Biochem (2010) 79:413-444; Young, T. S., et al., J Mol Biol (2010) 395:361-374).

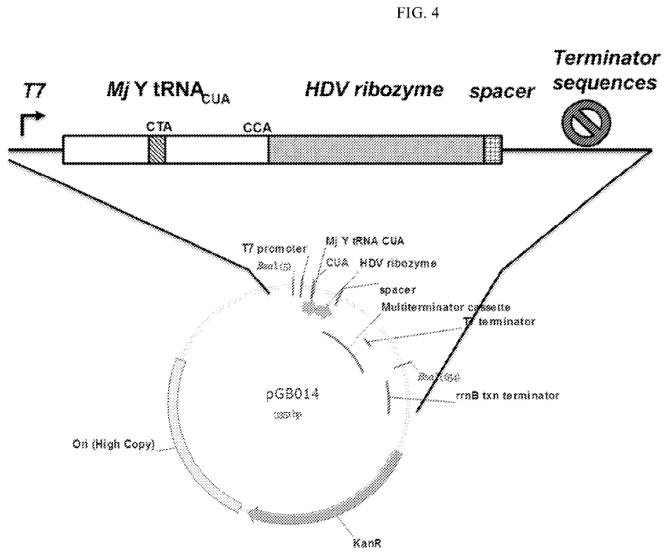

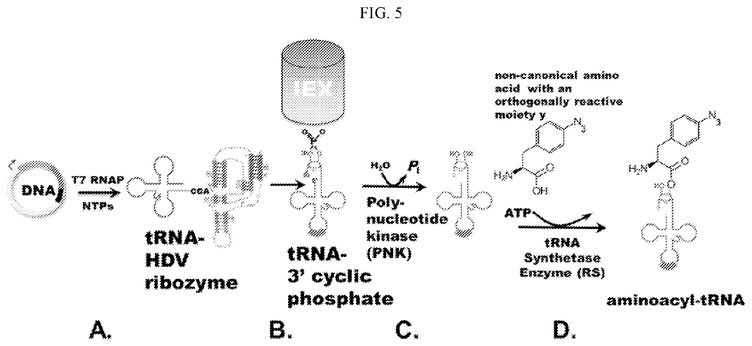

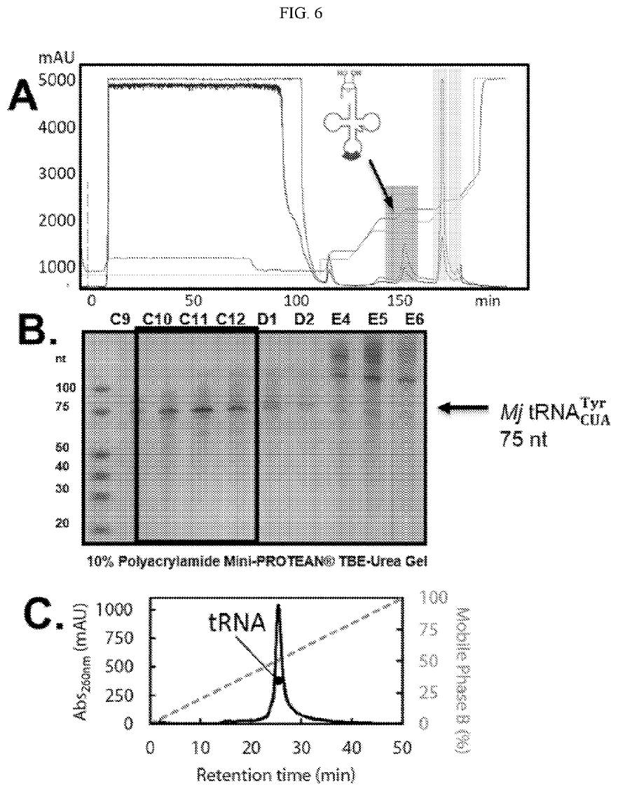

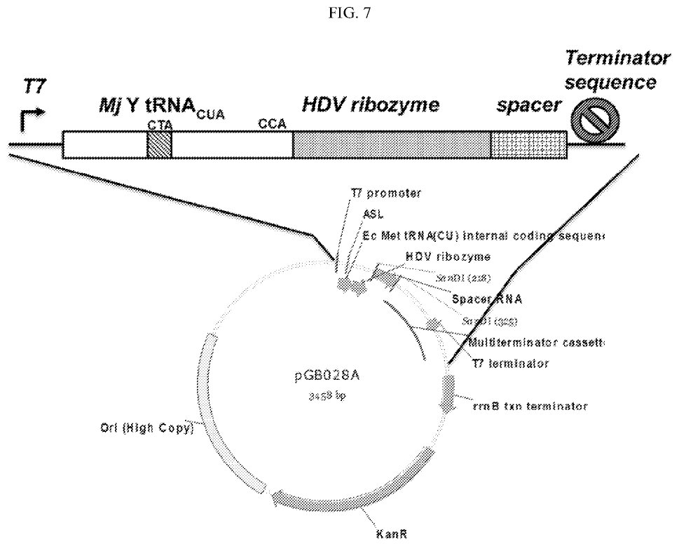

tRNA molecules to be used in the tRNA acylation reaction can be synthesized from a synthetic DNA template coding for any tRNA sequence of choice following amplification by PCR in the presence of appropriate 5′ and 3′ primers. Alternatively, a closed circular plasmid DNA template (FIG. 4 & FIG. 7), or rolling circle amplified plasmid DNA template can be used. The resulting double-stranded DNA template, containing a promoter sequence, can then be transcribed in vitro using, for example, T7 RNA polymerase to produce the tRNA molecule, which is subsequently purified (FIG. 5 & FIG. 6) or added to the tRNA acylation reaction. Alternatively, the tRNA may be chemically synthesized. In some embodiments the tRNA may be post-transcriptionally modified or produced in cells. In certain embodiments tmRNA may be produced from a DNA template sequence of choice following amplification by PCR in the presence of appropriate 5′ and 3′ primers.

In some embodiments, the ligand adduct moieties linked to canonical amino acids, α-hydroxyl acids, and non-canonical amino acids into a desired polymer, canonical amino acids, α-hydroxyl acids, and non-canonical amino acids are linked to the 3′ hydroxyl end of codon-independent tmRNAs via an acyl ester to form acylated tmRNA for subsequent formation of ligand adduct moiety acylated tmRNAs. Alternatively, the tmRNA acylation reaction occurs in the presence of a cell-free translation system, and as used in this invention, may include either canonical amino acids, α-hydroxyl acids, and non-canonical amino acids with an orthogonally reactive moiety. The tmRNA acylation reaction occurs in a separate reaction, where the charged tmRNA is then added to the cell-free translation reaction. Alternatively, the tmRNA acylation reaction occurs in the presence of a cell-free translation system.

The tRNA or tmRNA acylation reaction can be any reaction that acylates a selector codon reading tRNA molecule or codon independent tmRNA with a desired amino or α-hydroxyl acid separate from the protein synthesis reaction. This reaction can take place in a lysate, an artificial reaction mixture, or a combination of both. Suitable tRNA and tmRNA aminoacylation reaction conditions are well known to those of ordinary skill in the art as described in Francklyn, C. S., et al., Methods (2008) 44:100-118.

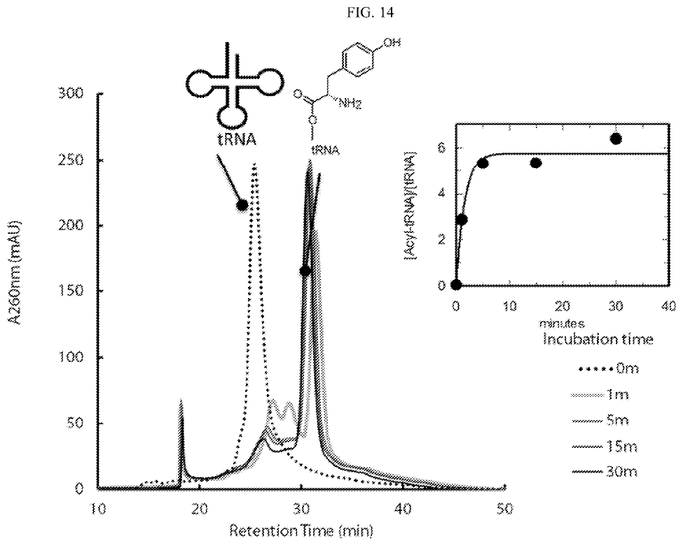

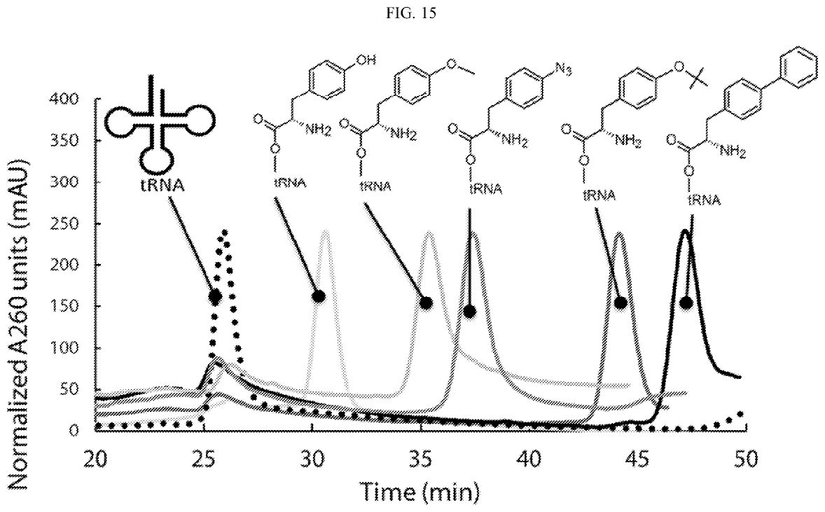

In other embodiments of the invention, selector codon reading tRNAs or codon independent tmRNAs are acylated by aminoacyl-tRNA synthetases (FIG. 14). The tRNA charging reactions can utilize either the natural aminoacyl-tRNA synthetase enzyme specific to the tRNAs to be acylated at the 3′ hydroxyl, engineered aminoacyl-tRNA synthetase, or a “promiscuous” aminoacyl tRNA synthetase capable of charging a tRNA molecule with more than one type of amino acid (FIG. 15). Typically, tRNA aminoacylation is carried out in a physiological buffer with a pH value ranging from 5.5 to 8.5, 0.5-10 mM high energy phosphate (such as ATP), 5-200 mM MgCl₂, 20-200 mM KCl. Preferably, the reaction is conducted in the presence of a reducing agent (such as 0-10 mM dithiothreitol). Where the aminoacyl-tRNA synthetase is exogenously added, the concentration of the synthetase is typically 1-20 μM. Promiscuous aminoacyl-tRNA synthetases with broadened substrate specificity through active site mutations may either themselves be engineered, or may include endogenously produced aminoacyl-tRNA synthetases that are sometimes found in nature. Engineered aminoacyl-tRNA synthetases are known in the art, and include but are not limited to, aminoacyl-tRNA synthetases with attenuated proofreading activity (Liu, C. C. and Schultz, P. G., Annu Rev Biochem (2010) 79:413-444; Datta, D., et al., J Am Chem Soc (2002) 124:5652-5653; Wang, L., et al., Science (2001) 292:498-500; Brustad, E., et al., Bioorg Med Chem Lett (2008) 18:6004-6006; Kiga, D., et al., Proc Natl Acad Sci USA (2002) 99:9715-9720). One skilled in the art would readily recognize that these conditions can be varied to optimize tRNA aminoacylation, such as high specificity for the pre-selected amino acids, high yields, and lowest cross-reactivity.

In still other embodiments of the invention, engineered RNA ribozymes known in the art (Flexizymes) may be used to produce acyl-tRNAs (Goto, Y., et al., Nat. Protocols (2011) 6:779-790). In some embodiments engineered nucleotidyl transferase enzymes may be used to produce acyl-tRNAs. In some embodiments, acyl-tRNAs may be produced from enzymatic ligation of chemically synthesized aminoacyl-RNAs with tRNA lacking 3′ RNA. In some embodiments the chemically synthesized aminoacyl-RNAs may be in contain 2′-deoxycytosine or a 2′ hydroxyl protecting group.

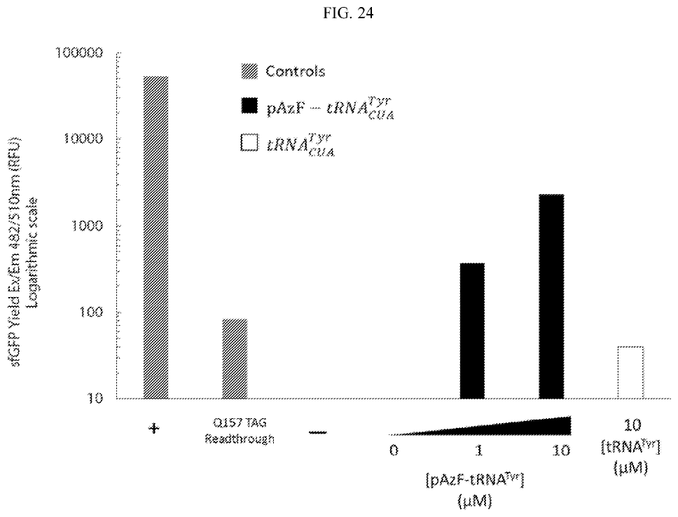

It will be appreciated that the inventive methods may also be used to synthesize other classes of chemical compounds besides polypeptides. For example, in some embodiments, acyl-tRNAs may be alpha-hydroxyl acyl-tRNAs capable of ribosome-directed translation to form an ester bond. Such alpha-hydroxyl acyl tRNAs may be prepared from certain aminoacyl-tRNAs by the action of oxidizing agents such as NaNO₂ (see FIG. 24; Fahnestock, S. and Rich, A., Science (1971) 173:340-343). Alternatively, alpha-hydroxyl acyl-tRNAs may also be prepared by engineered tRNA synthetases or by catalysis by ribozymes. Alternatively, alpha-hydroxyl-tRNAs may be prepared from T4 RNA ligase catalyzed ligation of hydroxyacyl-RNA with tRNA lacking 3′ residues.

In one embodiment of the invention, the acyl-tRNAs composition optionally includes at least about 10 micrograms, e.g., at least about 100 micrograms, at least about 1 milligram, at least about 10 milligrams, at least about 100 milligrams, or even about 1 gram or more of the acyl-tRNAs, e.g., an amount that can be achieved with in vivo RNA production methods (Perona, J. J., et al., J Mol Biol (1988) 202:121-126). For example, acyl-tRNA is optionally present in the composition at a concentration of at least about 10 milligrams per liter, at least about 50 milligrams per liter, at least about 100 milligrams per liter, at least about 500 milligrams per liter, at least about 1 gram per liter, or at least about 10 grams per liter, e.g., in a cell lysate, pharmaceutical buffer, or other liquid suspension (e.g., in a volume of, e.g., anywhere from about 1 mL to about 100 L). The production of large quantities (e.g. greater that that typically possible with other methods) of acyl-tRNA with canonical amino acids or α-hydroxyl acids, or non-canonical amino acids, or α-hydroxyl acids with orthogonally reactive moieties is a feature of the invention and is an advantage over the prior art.

### Acyl TRNA Non-Canonical Amino Acid Ligand Adducts

In order to encode ligand adducts linked to an amino or α-hydroxyl acid side chain described herein into a desired polymer, acyl-tRNAs with amino acids or α-hydroxyl acids containing an orthogonally reactive moiety y described above are reacted with a ligand with reactive moiety x, via a linker z, to form an acyl-tRNA non-canonical amino acid ligand adduct, tRNA-A-z-L, as illustrated for example in FIG. 3.

The assignment of reactive moieties between x and y may be determined by one skilled in the art based on considerations such as speed of reaction, absence of side reactions in the reaction mixture, reversibility of reaction, reactant and product stability, size, shape, hydrophobicity of the ligand, and conformational flexibility of the linker, etc., the person skilled in the art being able to decide (either experimentally or theoretically) without inappropriate effort whether a particular reaction is possible. Synthetic methods for forming a reversible or irreversible covalent bond between reactive moieties are well known in the art, and are described in basic textbooks, such as, e.g. March's Advanced Organic Chemistry, John Wiley & Sons, New York, 7^{th} edition, 2013; Larock, R. C., Comprehensive Organic Transformations: A Guide to Functional Group Preparations, Wiley-VCH; 2nd edition, 1999.

RNA differs chemically from DNA in two major ways. Firstly, it contains uracil instead of thymine, and secondly, RNA has a 2-—OH group on the ribose sugar instead of 2′-H found on the deoxyribose sugar of DNA. When RNA is manipulated for any number of common laboratory practices, its inherent instability is considered to lead to technical and experimental difficulties.

Modification of RNA chains using chemical reagents has been reported. Specific examples of modifying chemicals that have been used include dimethylsulphate leading to base modification (Bollack et al., (1965) Bull. Soc. Chim. Biol. 47:765-784), N-chlorosuccinimide leading to base modification and RNA degradation (Duval and Ebel, (1967) Bull. Soc. Chim. Biol. 49:1665-1678; Duval and Ebel., (1966) C.R. Acad. Sc. Paris t. 263:1773 series D), N-bromosuccinimide (Duval and Ebel, (1965) Bull. Soc. Chim. Biol. 47:787-806), diazomethane leading to methylation of the base and phosphate causing RNA breakdown (Kriek and Emmelot., (1963) Biochemistry 2:733), carbodiimide leading to base modification (Augusti-Tocco and Brown (1965) Nature 206:683), alkyl halides leading to base and phosphate modification (Ogilvie et al., (1979) Nucleic Acids Res. 6:1695), allyl bromide leading to guanine modification and chain degradation (Bollack and Ebel, (1968) Bull. Soc. Chim. Biol. 50:2351-2362), and hydroxylamine leading to cytosine modification (Verwoerd, D. W., Kohlhage, H, & Zillig, W. (1961) Nature, 192: 1038-1040; Brown, D. M. and Schell, P. (1965) J. Chem. Soc. 208-215). It has been reported that the use of acetic anhydride in DMF results in acylation of cytosine (Keith and Ebel (1968) C.R. Acad. Sc. Paris t. 266:1066 series D). Methyl sulphate has been used to modify the bases of an RNA template (Louisot et al., (1968) Annales de L'institut Pasteur. 98). Irreversible adsorption of RNA on metal surfaces or unspecific phosphodiester cleavage catalyzed by the metal ions is associated with nucleic acid damage in general and also with “hydroxyl radical footprinting”. See, for example, Handbook of RNA Biochemistry, Volume 1, edited by R. K. Hartmann, A. Bindereif, A. Schon, and E. Westhof, Wiley-VCH Verlag GmbH & Co. Weinheim, FRG (2005), pp 151. The results of such chemical modification reactions of RNA are therefore degradation, base and/or phosphate modification. In general, yields in these synthetic experiments have usually been low (10-60%), requiring high temperatures and vigorous conditions to modify RNA.

Thus, the chemoselective reactions of the reactive moieties of the claimed invention that do on affect RNA may appear difficult at first sight. We have found surprisingly that the supposed instability of RNA does not prevent one skilled in the art from modifying acyl-tRNAs to form acyl-tRNAs with ligand adduct moieties that are functional in ribosome-directed translation.

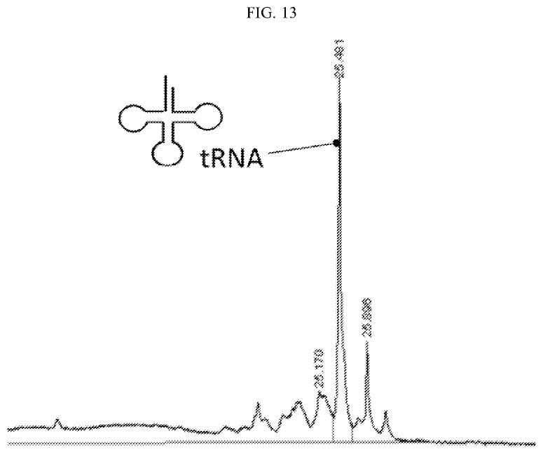

For example, copper(I)-catalyzed “click chemistry”, for which the reaction conditions were allegedly incompatible with RNA has been successfully utilized for chemoselective conjugation methods (Motorin, Y., et al., Nucleic Acids Research (2011) 39:1943-1952). It is well known in the art that tRNAs are stable to acidic pH. One skilled in the art will consider this stability in designing chemical reactions compatible with acidic pH, cf. FIG. 13 (Chapeville, F., et al., Proc Natl Acad Sci USA (1962) 48:1086-1092; Fahnestock, S. and Rich, A., Science (1971) 173:340-343; Peacock, J. R., et al., RNA (2014)). In some embodiments formation of ligand adduct moieties may be carried out in mixed aqueous solvents (Reuben, M. A., et al., Biochim et Biophys Acta (1979) 565:219-223).

In general, it is not material which chemically reactive moiety of a given pair is on the transfer RNA unit and which is on the ligand prior to subsequent reaction to form the aminoacyl-tRNA ligand adduct moieties. In general, it is not material whether mixtures of diastereomers, regioisomers, or enantiomers are formed in the ligand adduct moieties.

In some embodiments, ligand adduct structures can serve as substrates for additional chemical synthesis. For example, a ligand moiety x-L-q (see Scheme I, below) can contain two or more functional groups (“x” and “q”) suitable for performing synthetic organic chemistry. One functional group moiety x is used to synthesize ligand adduct moiety aminoacyl-tRNAs of the present invention, while the other functional group q may be used to incorporate additional reagents “B” pre-translationally or post-translationally. Alternatively, once a A-z-L-q moiety is confirmed as a hit ligand for a target of interest, small-molecule ligand structures such as A-z-L-q-Bₙ or L-q-Bₙ may be designed and screened for target affinity. The moiety q thus provides a direct avenue for subsequent chemical modification steps (e.g., increase of compound solubility, fragment assembly, or attachment of a payload).

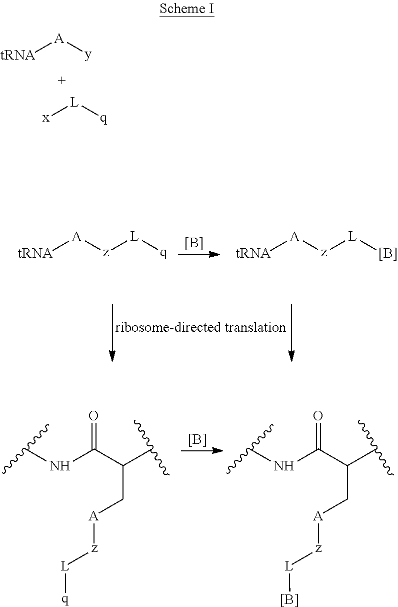

In some embodiments, the reaction between reactants can involve a further reactant, such as a “template-molecule”, mediating a connection between the two reacting moieties. In certain embodiments the linking reactions may be enzyme catalyzed.

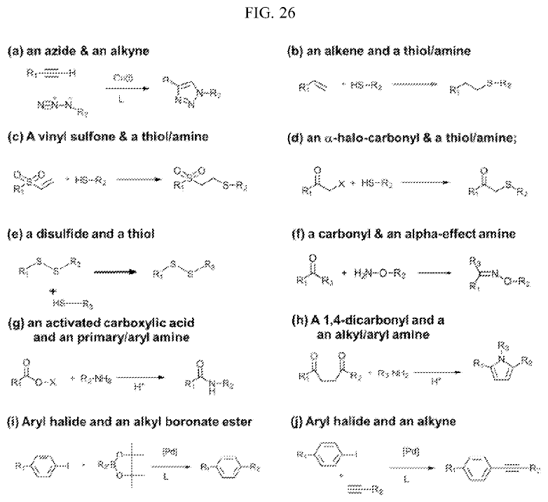

Representative reactive moieties and their reaction products are shown in FIG. 26 and described below.

### Linkers

### Azides and Alkynes

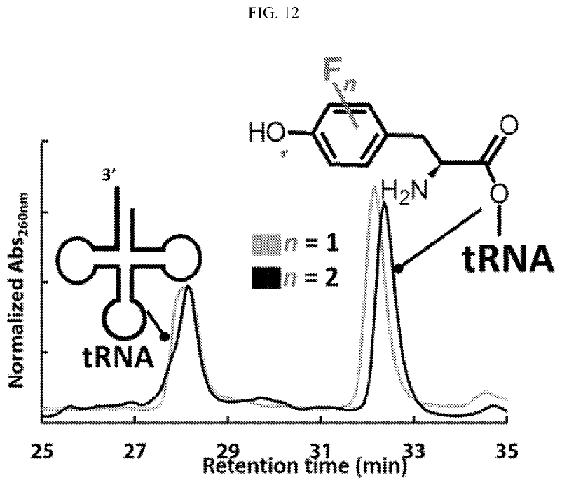

Reactions of azides with terminal alkynes R—C≡C—H (e.g., ethynyl group), termed [3+2] cycloaddition reactions, forming disubstituted triazoles (FIG. 12(a)), are within the skill of the art. In certain preferred embodiments, the [3+2] cycloaddition is performed under aqueous conditions. In embodiments where the 1,3-dipole is an azide and the dipolarophile is a terminal alkyne, the [3+2] cycloaddition may be performed as described by Sharpless and coworkers (V. V. Rostovtsev et al., Angew Chem. Int. Ed. Engl. (2002) 41: 1596-1599; W. G. Lewis et al., Angew Chem. Int. Ed. Engl. (2002) 41: 1053-1057; Wang et al., J. Am. Chem. Soc. (2003) 125: 3192-3193) at physiological temperatures, under aqueous conditions and in the presence of copper(I) (or Cu(I)), which catalyzes the cycloaddition. The Cu(I) catalyzed version of the [3+2] cycloaddition is termed “click” chemistry. In certain embodiments the [3+2] cycloaddition product triazole R₁ and R₂ groups may be syn or anti. [3+2] cycloaddition reactions can be carried out by the addition of Cu(II) (including but not limited to, in the form of a catalytic amount of CuSO₄) in the presence of a reducing agent for reducing Cu(II) to Cu(I), in situ, in catalytic amounts. See, e.g., Wang, Q., et al., J. Am. Chem. Soc. (2003), 125, 3192-3193; Tornoe, C. W., et al., J. Org. Chem., (2002), 67:3057-3064; Rostovtsev, et al., Angew. Chem. Int. Ed. (2002), 41:2596-2599. Exemplary reducing agents include, but are not limited to, ascorbate, metallic copper, quinine, hydroquinone, vitamin K, glutathione, cysteine, Fe²⁺, Co²⁺, and an applied electric potential. Reaction conditions that may be optimized include Cu(I) ligand structures (Besanceney-Webler, C., et al., Angew Chem Int Ed Engl (2011) 50:8051-8056), catalytic general acids and bases, reducing agents, pH, organic cosolvents, etc. In certain preferred embodiments a general acid or base catalyst may be used. The synthesis of azides and alkynes are well known in the art, cf. March's Advanced Organic Chemistry, John Wiley & Sons, New York, 7^{th} edition, 2013, Scriven, E. F. V. & Turnball, K. Chem. Rev., (1988), 88:297-368.

### Alkenes and Thiols or Amines

Strained alkenes such as unactivated dihydro alanine (Dha) display known reactivity with thiols and amines when embedded in peptide and protein sequences (Wang, J., Schiller, Schultz, P. G., Angewandte Chemie Int. Ed., (2007), 46: 6849-6851; Chatterjee, et al. Chem. Rev., (2005) 105:633-684.) In one embodiment, vinyl imine structures (Scheme II) may be generated from phenylselenocysteine acyl-tRNA, with the addition of H₂O₂ at room temperature for about one hour. Conversion to the tautomeric forms of the vinyl amine/methyl imine can be trapped in the presence of high concentrations of thiol nucleophiles in aprotic solvents to yield the β-thio or β-amino alkyl substituted alanyl acyl-tRNA. In the absence of trapping agents in protic solvents, the acetyl acyl-tRNA is generated. In some embodiments the acetyl acyl-tRNA may be removed from the reaction mixture by hydroxylamine affinity chromatography.

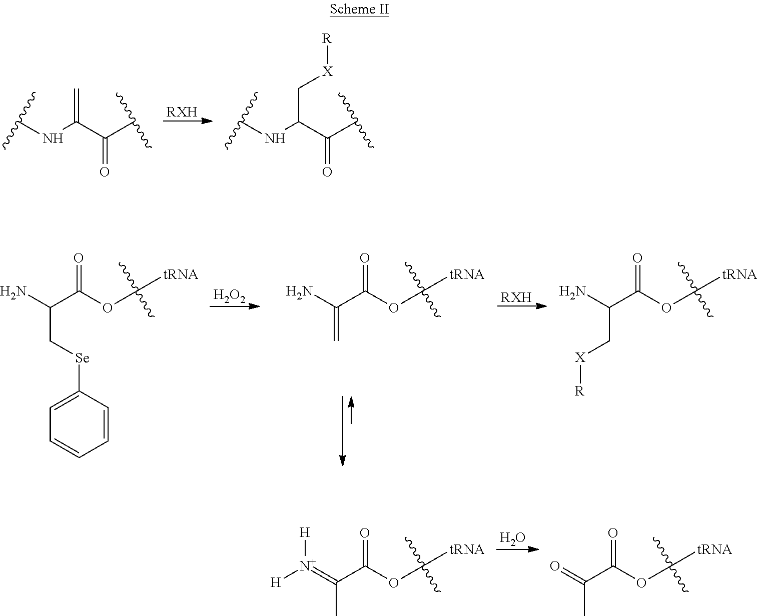

In some embodiments activated Michael acceptor acryloyl groups such as maleimides may be used as reactive alkenes. The maleimide group reacts specifically with sulfhydryl groups when the pH of the reaction mixture is between pH 6.5 and 7.5; the result is formation of a stable thioether linkage that is not reversible (i.e., the bond cannot be cleaved with reducing agents).

### Vinyl Sulfones and Thiols or Amines

Ligand adduct moieties may be formed by the reaction of vinyl sulfone moieties with thiols or amines to form β-heterosubstituted sulfones (FIG. 26 (c)). Vinyl sulfones are excellent Michael acceptors because of the electron poor nature of their double bond owing to the sulfone's electron withdrawing capability. Reaction conditions that may be optimized include the pKₐ and concentration of thiol nucleophiles, general acid and base catalysts, and the pH of the medium (Lutolf, M. P.; Tirelli, N.; Cerritelli, S.; Cavalli, L. & Hubbell, J. A., Bioconjugate Chem., (2001), 12:1051-1056.). The synthesis of vinyl sulfones are well known in the art, cf. Simpkins, N. S. Tetrahedron, (1990) 46: 6951-6984; Meadows, D. C. & Gervay-Hague, J. Med. Res. Rev., (2006) 26: 793-814.

### α-Halocarbonyls and a Thiol or an Amine

Ligand adduct moieties may be formed by the reaction of α-halomethyl carbonyls with thiols, and to a lesser extent amines, to form stable α-thioether carbonyls and α-aminomethyl carbonyls. In alpha-haloacetyl functional groups, the carbon halogen bond experiences increased polarity from the inductive effect of the carbonyl group making the carbon atom more electrophilic. Reaction conditions that may be optimized include the pKₐ and concentration of thiol or amine nucleophiles, addition of general acid and base catalysts, the pH of the medium, amphiphiles, micelles, etc. Side reactions to consider include alkyl halides that are known to react with RNA leading to base and phosphate modification (Ogilvie et al., (1979) Nucleic Acids Res. 6:1695) and allyl bromide leading to guanine modification and chain degradation (Bollack and Ebel, (1968) Bull. Soc. Chim. Biol. 50:2351-2362).

In some embodiments, the α-halomethyl carbonyl may be photoreactive, e.g. reaction of Cys-tRNA^{Cys} with 4-nitro benzyl acyl α-halomethyl reaction product may be photolyzed to yield the aldehyde product, as shown in Scheme III. The aldehyde acyl-tRNA may serve as an electrophile in the presence of alpha hydroxyamines, as described below. In some embodiments, the aldehyde group may exist as a hydrate, hemiacetal, or acetal. In some embodiments the amino group is protected so that the ribosome-directed translation of the ligand adduct acyl-tRNA may not take place until a desired time when the amine reactive group is are deprotected.

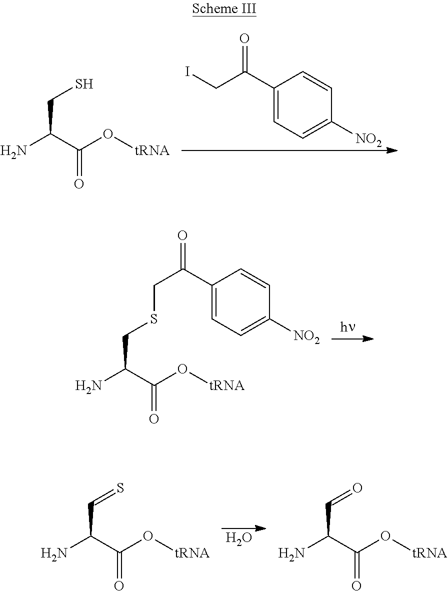

### Disulfide Exchange Reactions

Ligand adduct moieties may be formed though disulfide exchange between thiols and disulfides under reducing conditions (Witt, D. (2008), Synthesis, 16:2491-2509). In some cases activated disulfide reagents R₁—S—S—R₂ (FIG. 26(e)) with R₂=methanethiosulfonate, phenylthiosulfonate, or phenylselenenyl, cf. Davis, B. G. et al. J. Org. Chem. (1998) 63:9614-9615, or with R₂=pryidyl, react rapidly and specifically with thiols to provide mixed disulfides. Pyridyl disulfides react with sulfhydryl groups with a pH optimum of 4 to 5 and are preferred. The released product, pyridine-2-thione can be measured spectrophotometrically (Amax=343 nm) in order to monitor the progress of the reaction. Disulfide exchange reactions may be used to build combinatorial chemistry libraries of ligands Nicolaou, K. C., et al., Chemistry—A European Journal (2001) 7:4280-4295. Synthesis is slower at lower pH below the pKa of the attacking thiol, but is still feasible.

### Carbonyl and Alpha-Effect Amines

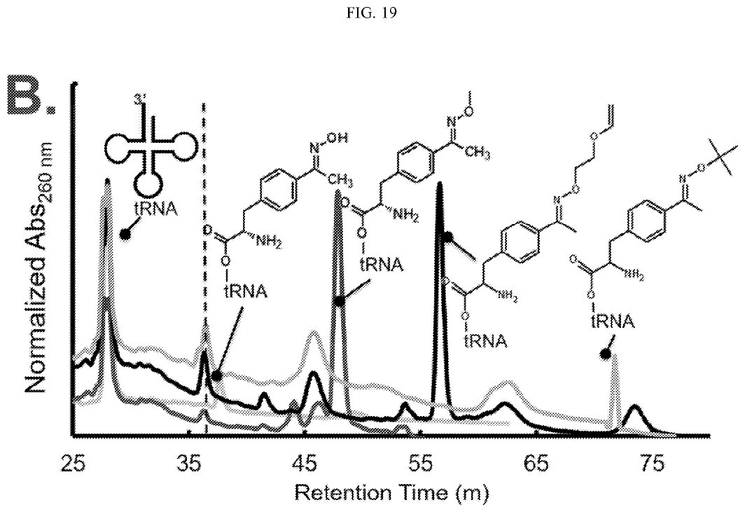

Ligand adduct moieties may be formed by the reaction of an alkyl or aryl hydroxyamine (an alpha-effect amine) with a carbonyl compound to produces an oxime, with the general formula RR′C═N—OR″ (FIG. 26(f)). The overall reaction involves nucleophilic attack by the hydroxyamine on the carbonyl group to give a carbinolamine. Dehydration then produces an oxime. Oximes possess greater intrinsic hydrolytic stability than do other imines or even hydrazones (Kalia, J. & Raines, R T, Angew Chem Int Ed Engl. 2008; 47: 7523-7526). In some embodiments where the carbonyl is an aldehyde or ketone and the alpha-effect amine is an alkyl hydroxylamine, the reaction may be performed as described by Jencks and coworkers. (Jencks W P. J. Am. Chem. Soc. 1959; 81:475-481; Anderson B M, Jencks W P. J. Am. Chem. Soc 1960; 82:1773-1777; Wolfenden R, Jencks W P. J. Am. Chem. Soc 1961; 83:2763-2768: Cordes, E H, Jencks, W P. J. Am. Chem. Soc 1962; 84:826-831; Cordes E H, Jencks W P. J. Am. Chem. Soc 1962; 84:832-837; Jencks W P. J. Am. Chem. Soc 1968 90:6154-6162; Sayer J M, Peskin M, Jencks W P. J. Am. Chem. Soc 1973; 95:4277-4287). Reaction conditions may be optimized by changing pH, amine and carbonyl concentration, temperature, organic co-solvents, etc. The potential side reaction of alkyl hydroxylamine with cytosine carbonyls and acyl ester aminolysis is avoided in this way (cf. FIG. 19).

In some embodiments the carbonyl compound may be masked or protected as vinyl ether, as for example, ethoxy vinyl glycine aminoacyl-tRNA may be formed (Scheme IV). Deprotection of the vinyl ether by general acid catalysis using alkyl phosphonic acids at low pH (Chwang, W. K., et al., J Am Chem Soc (1977) 99:805-808) yields the aldehyde functional group that may be trapped by high concentrations of ligand hydroxyamines, L-ONH₂. In certain embodiments, the free amine of acyl-tRNAs may be protected. In certain embodiments, the carbonyl group can exist in rapid equilibrium with its hydrate, as a hemiacetal, or as an acetal.

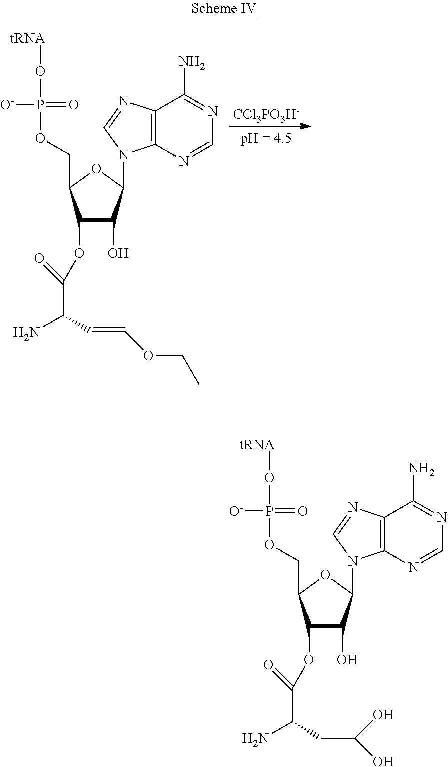

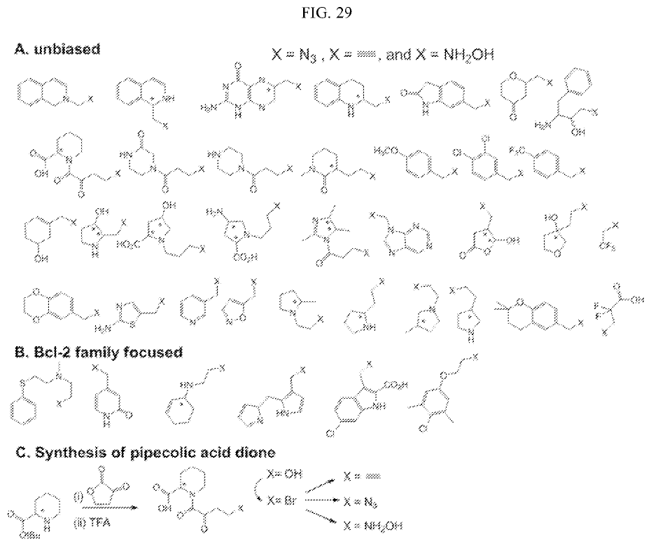

In one embodiment of the present invention, the chemically reactive group is either an aldehyde or ketone group and the library of ligand structures comprises primary hydroxyamines (FIG. 29A). In another preferred embodiment, the chemically reactive moiety is a primary hydroxyamine group and the library of ligand comprises candidate ligands with reactive aldehyde and/or ketone moieties.

In one embodiment once the oxime ligand adduct is formed between the aldehyde or ketone group and the hydroxyamine, the oxime bond created may optionally be reduced (i.e., made irreversible) by the addition of a reducing agent in order to stabilize the covalently bonded product of the reaction.

### Activated Carboxylic Acid Derivatives with Alkyl and Aryl Amines

Ligand adduct moieties may be formed by the reaction of an alkyl or aryl amine with a activated carboxylic acid derivative to produce an amide, with the general formula RR′C═O(—NHR″) (FIG. 26(g)). Formation of an amide from an activated carboxylic acid derivative (denoted RR′C═O(O—X)), effectively starts at a higher energy on the free energy reaction pathway. The nature of the leaving group ‘X’ governs the value of the activation energy. Representative activated carboxyl ester derivatives include anhydrides, pentafluorophenyl esters, and N-succinimidyl esters. Representative activated carboxylic acid derivatives include acid chlorides, acyl azides, and the like. Preferred primary amines include amines with low pKₐs<7, that may react at low pHs in preference to the aminoacyl nitrogen that is protonated at low pH, cf. Kajihara, D., et al., Nat Meth (2006) 3:923-929 or the amino groups of RNA purines and pyrimidines. In some embodiments the acylamine may be protected, cf. U.S. Pat. No. 7,288,372. In some embodiments the amine may be an alpha-effect amine.

### A 1,4-Dicarbonyl and a Primary Amine

The condensation of a 1,4-dicarbonyl compound with an excess of substituted primary amines is termed the Paal-Knorr synthesis of pyrroles (Ferreira et al., Organic Preparations and Procedures International, (2001) 33:413-466). The N-substituted pyrrole structure is a common scaffold for small molecule drug design. The reaction can be carried out under mild conditions with a variety of substituted amines for diversity oriented synthesis (Werner et al. J Comb Chem. (2006) 8:368-380). Variables that are optimized include pH (the reaction is acid catalyzed), general acids, Lewis Acids, amine concentration, temperature, etc.

### Aryl Halide and Alkyl Boronate Esters

In some embodiments ligand adducts may be formed by reaction of aryl halides and alkyl boronate esters in the presence of palladium (Pd) ligand catalysts, termed the Suzuki-Miyaura cross coupling reaction. In certain preferred embodiments, the Suzuki-Miyaura reaction is performed under aqueous conditions. In embodiments where the aryl halide is a derivative of phenylalanine iodide and the boronate ester is an alkyl boronate ester, the reaction may be performed as described by Chalker, Wood, and Davis, B. G., J. Am. Chem. Soc., (2009), 131, 16346-16347 at physiological temperatures, under aqueous conditions in the presence of Palladium(I) which catalyzes the cross coupling. In some embodiments the aryl group may be a heteroarene, which follows the Hückel 4n+2 rule. Reaction conditions that may be optimized include Pd(I) ligand structures (Lercher, L., McGouran, J. F., Kessler, B. M, Schofield, C. M., and Davis, B. G., Angew. Chem. Int. Ed., (2013), 52, pp 10553-10558), general acids and bases, reducing agents, pH, organic cosolvents, nonionic amphiphiles, temperature, etc. In this case one of skill in the art may design the boronate ester ligand structures to prevent potential reaction with 2,3 cis-diols of tRNA.

### Aryl Halide and an Alkyne

The reaction of aryl or vinyl halides with a terminal alkyne R—C≡C—H (e.g., ethynyl group), catalyzed by palladium is termed the Sonogashira reaction. It is a cross-coupling reaction used in organic synthesis to form carbon-carbon bonds. The reaction can be carried out under mild conditions, such as at room temperature, in aqueous media. Because of the inherent rigidity of the aryl alkyne linker formed, ligand adducts will have a well-defined position in 3-dimensional space when displayed on various polypeptide secondary structures, such as alpha helices and beta-sheets. This structural information is encoded in the mRNA sequence, and may be useful in small-molecule drug design and modeling.

### Candidate Ligands

A plurality of candidate ligands comprises a library of candidate ligands. In one embodiment, the library comprises at least 8 candidate ligands. In another embodiment, the library comprises at least 24 candidate ligands. In another embodiment, the library comprises at least 96 candidate ligands. In another embodiment, the library comprises at least 384 candidate ligands. In another embodiment, the library comprises at least 1,536 candidate ligands. In another embodiment, the library comprises at least 6,144 candidate ligands. In another embodiment, the library comprises at least 96,000 candidate ligands.

The library of candidate ligands, L, with reactive moiety, x, to be linked to acyl-tRNAs to form ligand adduct moiety acyl-tRNAs, may be obtained in a variety of ways including, for example, through commercial (e.g. Enamine LLC, Monmouth Junction, N.J. and Iota Pharmaceuticals, Cambridge, UK) and non-commercial sources, by synthesizing such compounds (see, for example, FIG. 29C) using standard chemical synthesis technology or combinatorial synthesis technology (see Gallop et al., J. Med. Chem. (1994) 37:1233-1251, Gordon et al., J. Med. Chem. (1994) 37:1385-140, Czarnik and Eliman, Acc. Chem. Res. (1996) 29:112-170, Thompson and Ellman, Chem. Rev. (1996) 96:555-600 and Balkenhohl et al. Angew. Chem. Int. Ed. (1996), 35:2288-2337; Srinivasan, R., et al., Org Biomol Chem (2009) 7:1821-1828; Lau, W., et al., Journal of Computer-Aided Molecular Design (2011) 25:621-636). For example, disulfide containing small molecule libraries may be made from commercially available carboxylic acids and protected cysteamine (e.g., mono-BOC-cysteamine) by adapting the method of Parlow et al., Mol. Diversity (1995) 1:266-269. Ligands may be obtained as degradation products from larger precursor compounds, e.g. known therapeutic drugs, large chemical molecules, and the like.

The ligands of this invention may be biased to appropriate moieties to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

In some embodiments, the ligands may be biased towards specific chemical structures likely to have strong affinity to a target, or weak affinity to an anti-target, based on known three-dimensional structures of targets and anti-targets, as for example in FIG. 29B. The ligands and ligand adduct moieties may contain one or more asymmetric centers and thus give rise to enantiomers, diastereomers, and other stereoisomeric forms that may be defined, in terms of absolute stereochemistry, as (R)- or (S)-, or as (D)- or (L)- for amino acids, or regioisomers, as in the case of triazole linkers, for example. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 15 1981). When the compounds described herein contain olefinic double bonds, other unsaturation, or other centers of geometric asymmetry, and unless specified otherwise, it is intended that the compounds include either E and Z geometric isomers (or cis- and trans-isomers). Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon heteroatom double bond depicted arbitrarily herein as trans may be cis, trans, or a mixture of the two in any proportion.

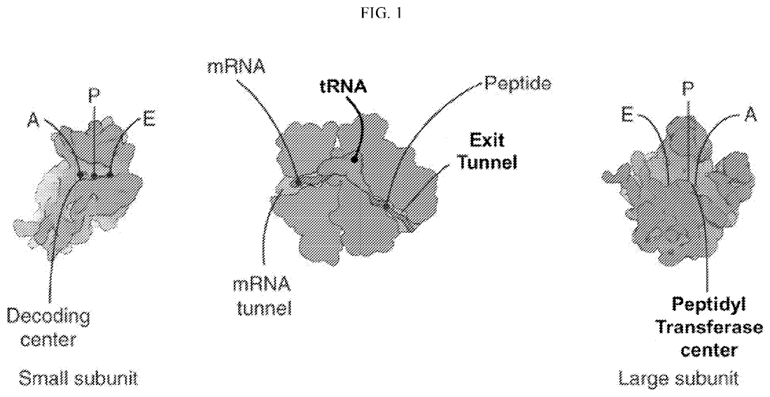

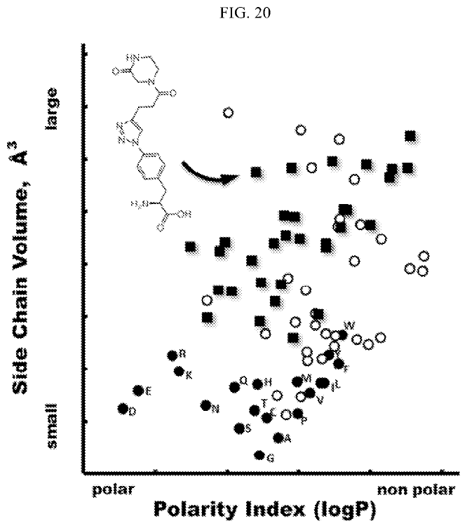

An important aspect of the invention is to use candidate ligand adducts that are capable of ribosome-directed translation (FIG. 1) in the form of acyl-tRNAs (FIG. 2) with non-canonical amino acid ligand adducts (FIG. 3). Based on the known distribution of canonical amino acid side chain properties such as hydrophobicity (Chothia, C. and Janin, J., Nature (1975) 256:705-708), polarity, and size (Zamyatnin, A. A., Prog Biophys Mol Biol (1972) 24:107-123), one of skill in the art may calculate amino acid side chain properties for ligand adducts from structural models using standard numeric methods (Lee, B. & Richards, F. M. J. Mol. Biol. 1971, 55, 379-40; Shrake, A. & Rupley, J. A. J. Mol. Biol. (1973)) 79, 351-371; Connolly, M. L. 1983, Science 221, 709-713; Richmond, T. J. J. Mol. Biol. (1984), 178, 63-89). Computer programs or algorithms are useful in the design of such ligand adduct moieties. Such calculations can serve to determine the likelihood that a ligand adduct moiety will be competent for ribosome-directed translation (FIG. 20).

In some embodiments, in order to determine that a ligand adduct moiety will be competent for ribosome-directed translation, octanol/water partitioning coefficients (log P and or log D) and van der Waals volumes or molecular surface areas of ligand adducts A-z-L are calculated using computer programs e.g. Chemaxon and ACD Chemsketch software. In some embodiments, the van der Waals volume of side chain ligand adduct moieties is less than about 100, 150, 200, 250, 300, 400 or 500 Å³. In some embodiments, the van der Waals volume is between 150 and 300 Å³.

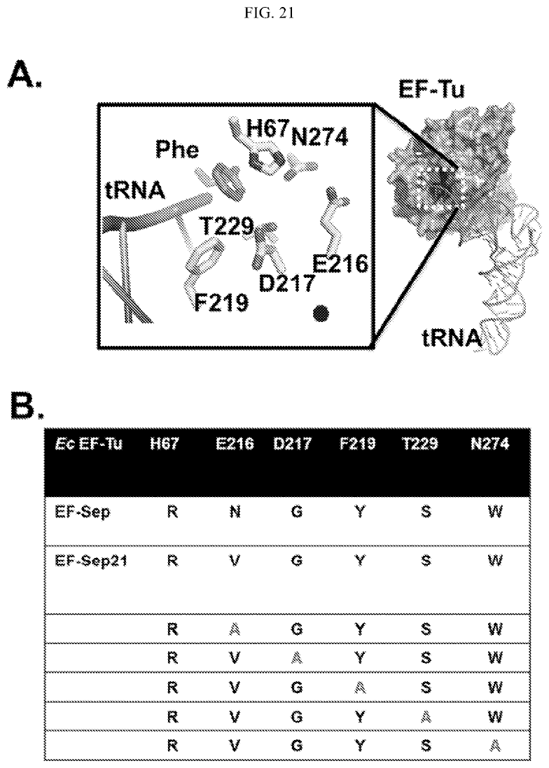

Since it is known that EF-Tu acyl-tRNA interactions are a component of quality control in protein synthesis (LaRiviere, F. J., et al., Science (2001) 294:165-168), in some embodiments, engineered variants of Ef-Tu may be designed in silico, screened, or selected to facilitate ribosome-directed translation of ligand adduct moieties, see for example, Park, H-F. and Soll, D. United States Patent Application 2013/0203112 incorporated herein as reference, and FIG. 21. In some embodiments tRNAs may be engineered to have high affinity to Ef-Tu in order to increase the efficiency of ribosome-directed translation by methods well known in the art. (Harrington, K. M., et al., Biochemistry (1993) 32:7617-7622).

In some embodiments, non-functional ligand adduct moieties of the library are removed by selecting a scaffold protein for display of library members, such that the scaffold protein is pre-selected for folded and functional molecules, see, e.g. U.S. Pat. No. 6,846,634, incorporated here by reference. For example, the scaffold protein may be half of an enzyme/ligand complex, an antibody (in the form of a monoclonal antibody or a polyclonal mixture of antibodies), or a secondary structure as an alpha-helix, a beta sheet, or beta-turn. The scaffold protein consists of a constant secondary or tertiary structure or sequence, which structure is liable to be absent or altered in non-functional members of the library. In the case of antibody libraries, this method is of use to select from a library only those functional members which have a binding site for a given target, such an approach is useful in selecting functional ligand adduct polypeptides. In the case of enzyme-displayed libraries, the members of the library that are functional may be assayed by enzyme activity. In some embodiments, the library members may be enriched for folded scaffold proteins by binding the library to an immobilized active-site ligand. In some embodiments the scaffold protein may be engineered to have a minimal codon set (Walter, K. U., et al., J. Biol. Chem. (2005) 280:37742-37746).

### Translation Systems

The ligand adduct acyl-tRNAs and molecules of the present invention can be placed in a translation system comprising a ribosome and associated factors and messenger RNA (mRNA) under conditions suitable for a peptidyl transferase reaction, thereby synthesizing a biopolymer incorporating the amino acid, alpha-hydroxyl acids, non-canonical amino acids, or non-canonical amino acid ligand adduct moieties. Translation systems may be cell-free or cellular, and may be prokaryotic or eukaryotic.

The translation system comprises macromolecules including RNA and enzymes, translation ribosomes, initiation and elongation factors, amino acids, and chemical reagents. RNA of the system is required in three molecular forms, ribosomal RNA (rRNA), messenger RNA (mRNA) and transfer RNA (tRNA). mRNA carries the instructions for building a polypeptide encoded within each selector codon sequence. In some embodiments tmRNA and mutated tmRNAs may be added. In some embodiments the mRNA may be modified. In some embodiments, the mRNA sequence may contain a barcode sequence.

In one embodiment, the translation system comprises a cell-free translation system. Cell-free translation systems are commercially available and many different types and systems are well-known (Promega, Madison, WS; Life Technologies, Carlsbad, Calif.). Examples of cell-free systems include prokaryotic lysates such as Escherichia coli lysates as described by Zawada, J. F., et al., Biotechnol Bioeng (2011) 108:1570-1578, and eukaryotic lysates such as wheat germ lysates, insect cell lysates, rabbit reticulocyte lysates, frog oocyte lysates, and human cell lysates. In some embodiments the growth rate of cells prior to lysis is optimized by procedures well known in the art for maximum recovery of active ribosomes. In some embodiments the cell-free lysate may be in the form of a dry powder, a lyophilisate, or produced by microwave assisted drying in a low pressure vacuum environment. In some embodiments, combinations of lysates or lysates supplemented with purified enzymes or proteins such as initiation factor-1 (IF-I), IF-2, IF-3 (alpha or beta), elongation factor T (EF-Tu), termination factors, or tRNA synthetase enzymes, may be used. It will be appreciated by those of skill in the art that such purified enzymes or proteins may be engineered for optimizing the efficiency of ribosome-directed translation of ligand adduct moieties.

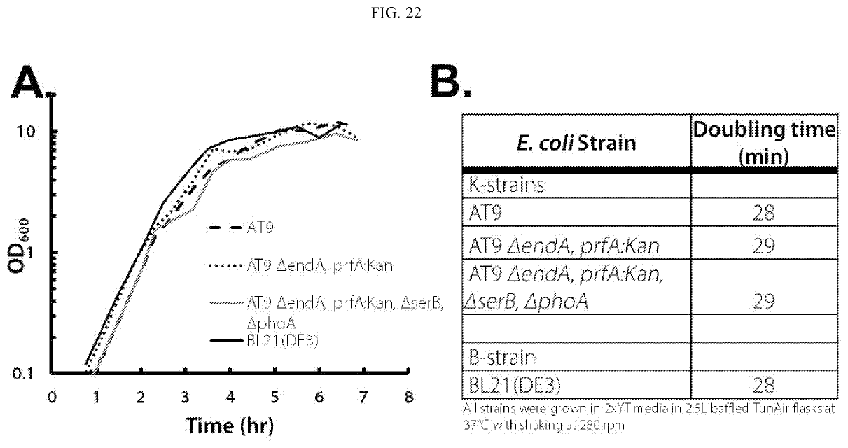

In some embodiments, translation systems may be engineered for efficient translation of amino acids with ligand adduct moieties by genome engineering of cell strains including engineering ribosomal RNA and ribosomal protein sequences Neumann, H., et al., Nature (2010) 464:441-444 (FIG. 22). In some embodiments the cell lysate may be produced from cell strains that are auxotrophic for certain amino acids. In some embodiments, the cell lysate may be conditionally activated by cell lysis and proteolysis of undesired translational proteins.

Translation mixes may comprise buffers such as Tris-HCl, HEPES, or another suitable buffering agent to maintain the pH of the solution between about 6 to about 8.5, and preferably at about 7. In some embodiments, the pH of the cell-free reaction may be controlled to facilitate ribosome-directed translation of acyl-tRNAs with non-canonical amino acids with ligand adducts; see, e.g (Wang, J., et al., ACS Chem Biol (2014)). Other reagents which may be in the translation system include dithiothreitol (DTT), 2-mercaptoethanol, cysteine, or glutathione as reducing agents and oxidizing agents, RNasin to inhibit RNA breakdown, RecBCD GamS protein to protect linear DNA template, nucleoside triphosphates or creatine phosphate and creatine kinase to provide chemical energy for the translation process, or Mg²⁺ ions, polyethylene glycol, and various ratios of canonical amino acids may be added.

Cell-free systems may also be transcription/translation systems wherein DNA is introduced to the system, transcribed into mRNA, and the mRNA translated by the ribosome. In embodiments wherein a DNA template is used to drive in vitro polypeptide synthesis, the individual components of the synthesis reaction mixture may be mixed together in any convenient order. Optionally, an RNA polymerase is added to the reaction mixture to provide enhanced transcription of the DNA template. RNA polymerases suitable for use herein include any RNA polymerase that functions in the bacteria from which the bacterial lysate is derived. In some embodiments modified ribonucleodides are used for transcription. In some embodiments the DNA template is from rolling-circle amplified DNA.

In embodiments wherein an RNA template is used to drive in vitro protein synthesis, the components of the reaction mixture can be admixed together in any convenient order, but are preferably admixed in an order wherein the RNA template is added last. mRNA molecules may be prepared or obtained from recombinant sources, or purified from other cells by procedure such as poly-dT chromatography, or by transcription of a DNA template in the presence of the cell-free lysate RNA transcribed in eukaryotic transcription system may be in the form of heteronuclear 5 RNA (hnRNA) or 5′-end caps (7-methyl guanosine) and 3′-end poly A tailed mature mRNA, which can be an advantage in certain translation systems. For example, capped mRNAs are translated with high efficiency in the reticulocyte lysate system. In some embodiments, the mRNA sequence may contain a spacer sequence that is fused in frame to an mRNA sequence of interest. In some embodiments the mRNA template may be a template designed for efficient ribosome display. In some embodiments the mRNA template may be a template designed for efficient mRNA display. In some embodiments, the mRNA template may be stabilized by the use of non-natural ribonucleotides. Some embodiments the mRNA template may be fused to a puryomycin linked oligo and the like. In some embodiments the mRNA template may be designed for efficient in vitro translation (Li, G. W., et al., Nature (2012) 484:538-541; Zawada, J. F., et al., Biotechnol Bioeng (2011) 108:1570-1578; Voges, D., et al., Biochemical and Biophysical Research Communications (2004) 318:601-614).

As will be appreciated by one of skill in the art, in some embodiments, the mRNA sequence may contain a unique sequence, or barcode in order to identify the chemical structure and sequence of ligand adduct moieties in a ribosomally synthesized polypeptide (a “molecular address”). The unique barcode sequence may be coding or non-coding. In some embodiments the barcodes may contain synonymous codons. Examples of unique barcode sequences may be found, for example, in Barendt, P. A., et al., ACS Chemical Biology (2013) 8:958-966. A 5 nucleotide sequence barcode allows for N⁵ (N corresponds to A/C/G/T)=4⁵=1024 different unique molecular addresses. A 10 nucleotide sequence barcode allows about 1000000 unique molecular addresses, and so on. Barcode sequences may be flanked with adaptor sequences, so that they can processed together in the same strategy as all other mRNAs under investigation; the unique adaptor sequence proceeds through the whole process. The molecular barcode therefore needs the same features as the molecules under investigation, so that they can be processed simultaneously. Several barcode sequence indices are available commercially, e.g. Illumina® Index (barcode). Sequence barcodes may be designed based on consideration of biological, sequencing, and code principles, e.g. Hamming codes.

Reconstituted mixtures of purified translation factors may be used to translate mRNA into protein as well (Shimizu, Y., et al., Nat Biotechnol (2001) 19:751-755; Forster, A. C., et al., Anal Biochem (2004) 333:358-364, U.S. Pat. No. 6,977,150, incorporated herein as reference). Reconstituted translation systems are essentially free of contaminating exonucleases, RNases, and proteases, and various factors such as amino acids, tRNAs, and release factors may be added or subtracted from the reaction mixtures in order to optimize the system (Schlippe, Y. V., et al., J Am Chem Soc (2012) 134:10469-10477).

Translations in cell-free synthesis systems generally require incubation of the ingredients for a period of time. Incubation times range from about 5 minutes to many hours, but are preferably between about thirty minutes to about twenty-four hours and more preferably between about one to about five hours. Incubation may also be performed in a continuous manner whereby reagents are flowed into the system and nascent proteins removed or left to accumulate using a continuous flow system (Spirin, A. S., et al., Science (1988) 242:1162-1164). Incubations may also be performed using a dialysis system where consumable reagents are available for the translation system in an outer reservoir which is separated from larger components of the translation system by a dialysis membrane (Kim et aI. (1996) Biotechnol Prog 12, 645-649). Incubation times vary significantly with the volume of the translation mix and the temperature of the incubation. The reaction mixture can be incubated at any temperature suitable for the transcription and/or translation reactions. Incubation temperatures can be between about 4° C. to about 60° C., and are preferably between about 15° C. to about 50° C., and more preferably between about 25° C. to about 40° C., and even more preferably at about 25° C. to about 32° C.

In some embodiments acyl-tRNAs with non-canonical amino acids with ligand adducts can be added at between about 1 microgram/mL to about 1.0 mg/mL, preferably at between about 10 microgram/mL to about 100 microgram/mL, and more preferably at about 20 microgram/mL. In some embodiments acyl-tRNAs with non-canonical amino acids with ligand adducts can be added at a single concentration at the beginning of a cell-free synthesis reaction. In some embodiments, acyl-tRNAs with non-canonical amino acids with ligand adducts can be added once during the cell-free synthesis reaction, intermittently, or at controlled rates of addition, or in a continuous manner.

The reactions may utilize a large scale reactor, small scale reactors (Siuti, P., et al., Lab on a chip (2011) 11:3523-3529), microfluidic based reactors (Squires, T. M. and Quake, S. R., Reviews of Modern Physics (2005) 77:977-1026), emulsion droplet reactors, bead reactors, or even at the single molecule level (Wen, J. D., et al., Nature (2008) 452:598-603). Reactions may be multiplexed or spatially addressed to perform a plurality of simultaneous polypeptide syntheses. Methods such as ‘Protein In Situ Array’ (PISA), nucleic acid programmable protein arrays (NAPPA; Ramachandran, N., et al., Science (2004) 305:86-90), or DNA array to protein array (DAPA; He, M., et al., Nat Methods (2008) 5:175-177) are well known in the art and may be used with ligand adduct acyl-tRNAs.

The reaction mixture can be agitated or unagitated during incubation. The use of agitation enhances the speed and efficiency of protein synthesis by keeping the concentrations of reaction components uniform throughout and avoiding the formation of pockets with low rates of synthesis caused by the depletion of one or more key components. The reaction can be allowed to continue while protein synthesis occurs at an acceptable specific or volumetric rate, or until cessation of protein synthesis, as desired. The optimal interval for allowing the in vitro translation reaction to proceed can be determined by assaying the yield of polypeptide. In some embodiments, the optimal interval for allowing the in vitro translation reaction to proceed can be determined by assaying the yield of ribosome-linked mRNA-polypeptide complexes, or mRNA- -polypeptide complexes, e.g., by recovery and real-time-PCR (RT-PCR). The reaction can be conveniently stopped by incubating the reaction mixture on ice. The reaction can be maintained as long as desired by continuous feeding of the limiting and non-reusable transcription and translation components.

In some embodiments of the invention, cell-free protein synthesis is performed in a reaction where the redox conditions in the reaction mixture are optimized. This may include addition of a redox buffer to the reaction mix in order to maintain the appropriate oxidizing environment for the formation of proper disulfide bonds or addition of chaperone proteins (Dsb system of oxidoreductases and isomerases, GroES, GroEL, DnaJ, DnaK, Skp, etc.) which may be exogenously added to the reaction mixture or may be overexpressed in the source cells used to prepare the cell lysate (Groff, D., et al., MAbs (2014) 6:671-678). The reaction mixture may further be modified to decrease the activity of endogenous molecules that have deleterious activity. Preferably such molecules can be chemically inactivated prior to cell-free protein synthesis by treatment with compounds that irreversibly inactivate free sulfhydryl groups, or removed entirely from the genome using methods well known in the art. The presence of endogenous enzymes having reducing activity may be further diminished by the use of lysates prepared from genetically modified cells having inactivation mutations in such enzymes, for example thioredoxin reductase, glutathione reductase, etc.

Lysates may be prepared by conditionally inactivated release factors whereby essential factors required for cell growth are maintained. Upon cell lysis to produce cell-free lysates, these factors may be degraded. Alternatively, such factors can be removed by genome editing methods known in the art (Johnson, D. B., et al., Nat Chem Biol (2011) 7:779-786; Wang, H. H. and Church, G. M., Methods in enzymology (2011) 498:409-426), or by selective removal from the cell-free lysate during its preparation, or by the addition of inhibitors.

In one embodiment, the acyl-tRNAs with non-canonical amino acids with ligand adducts of the present invention can be introduced into a cellular translation systems where they function in protein synthesis to incorporate ligand adduct moieties in the growing peptide chain. The cellular translation systems may be selected from the group consisting of tissue culture cells, primary cells, cells in vivo, isolated immortalized cells, human cells, cell organelles, cell envelopes and other discrete volumes bound by an intact biological membrane which contain a protein synthesizing system, and combinations thereof. Cellular translation systems include whole cell preparations such as permeabilized cells or cell cultures wherein a desired nucleic acid sequence can be transcribed to mRNA and the mRNA translated.

Acyl-tRNAs with non-canonical amino acids with ligand adducts can be introduced into cellular translation systems by a variety of methods that have been previously established, such as sealing the tRNA solution into liposomes or vesicles which have the characteristic that they can be induced to fuse with cells. The fusion of cells is used to refer to the introduction of the liposome or vesicle interior solution containing the tRNA into the cell. Alternatively, some cells will actively incorporate liposomes into their interior cytoplasm through phagocytosis. The tRNA solution could also be introduced through the process of cationic detergent mediated lipofection or injected into large cells such as oocytes. Injection may be through direct perfusion with micropipettes or through the method of electroporation. Alternatively, cells can be permeabilized by incubation for a short period of time in a solution containing low concentrations of detergents in a hypotonic media. Useful detergents include Nonidet-P 40 (NP40), Triton X-100, or deoxycholate at concentrations of about 1 nM to 1.0 mM, preferably between about 0.1 microM to about 0.01 mM, and more preferably about 1 microM.

In certain embodiments, cell-free synthesis reactions comprise at least one tRNA/tRNA synthetase pair with an non-canonical amino acid, where the tRNA base pairs with a selector codon. The tRNA synthetase may be exogenously synthesized and added to the reaction mix prior to initiation of polypeptide synthesis. The tRNA may be synthesized in the cells from which the cell lysate is obtained, may be synthesized in situ during the transcription reaction, or may be exogenously added to the reaction mix.

The recovery of polypeptides produced by the translation system may be facilitated by the use of various “tags” that are in the translated polypeptide which bind to specific substrates or molecules. Numerous reagents for capturing such tags are commercially available, including reagents for capturing the His-tag, FLAG-tag, glutathione-S-transferase (GST) tag, strep-tag, HSV-tag, T7-tag, S-tag, DsbA-tag, DsbC-tag, Nus-tag, nano-tag, myc-tag, hemagglutinin (HA)-tag, Trx-tag (Novagen, Gibbstown, N.J.; Pierce, Rockford, Ill.), or SUMO fusion-tag (Lucigen, Middleton, Wis.). Alternatively, the translated polypeptide may be recovered by chromatography media (ion exchange, affinity resins) and the like. In some embodiments the cell-free lysate may be engineered to facilitate cleavage of the tag under desired conditions.

### Ligand Adduct Libraries

To achieve high diversity with low molecular weight (M.W. ˜100-˜1000 AMU) canonical amino acid side chains and non-canonical side chain ligand adduct libraries that are suitable for drug discovery, about 100 different building blocks embedded into a biopolymer sequence length of >6 may be required (Chothia, C. and Janin, J., Nature (1975) 256:705-708), corresponding to >10^{2x6} ligand adduct acyl-tRNAs or >10¹² chemical diversity. Because ribosome concentrations in cell-free translation systems are about >10¹⁴/mL (Pluckthun, A., Ribosome Display and Related Technologies: Methods and Protocols (2012) 805:3-28), a diverse library of >10¹² would require a translation reaction larger than 100 mL. At normal in vitro protein synthesis concentrations (up to ˜2 milligram/mL acyl-tRNA), a considerable amount of tRNA is necessary (up to ˜200 milligrams). In vitro synthesis through ribosomes is critical because it allows for the encoding and decoding of large chemical diversities as well as using in vitro selection and evolution methods well known in the art. (Mattheakis, L. C., et al., Proc Natl Acad Sci USA (1994) 91:9022-9026; Stafford, R. L., et al., Protein Eng Des Sel (2014); Methods in Molecular Biology (2012) 805; Frankel, A., et al., Current Opinion in Structural Biology (2003) 13:506-512). In some embodiments the library is an mRNA-protein fusion library. In some embodiments, ligand adduct libraries may be fused to an unstructured polymer sequence such as recombinant PEG sequences. In some embodiments, the ribosome translation mix is a reconstituted mix.

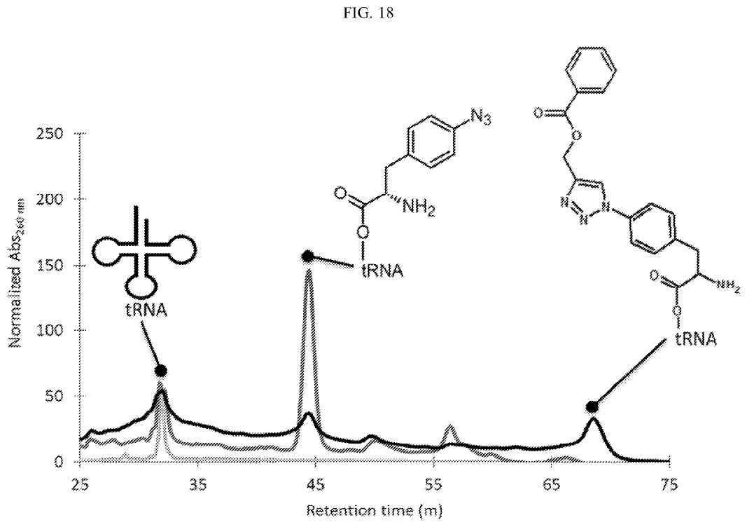

As will be appreciated by those of skill in the art, there are several advantages to chemical library screening using the method described herein. First, libraries synthesized in this manner (i.e., having been encoded by a nucleic acid) have the advantage of being amplifiable and evolvable in vitro as described by Wrenn, S. J. and Harbury, P. B., Ann Rev Biochem (2007) 76:331-349. The basic procedure, outlined in FIG. 18, is to use iterative rounds of transcription/translation, pooling ribosomal complexes for selection against a target, amplification and sequencing of recovered DNA, where the selective step increases the proportion of functional molecules, and amplification increases their number. With each round, the library is enriched in molecules that satisfy the selective criteria. Thus, an originally diverse population that may contain only a single copy of a desirable molecule quickly evolves into a population dominated by the molecule of interest. The amplified nucleic acid can then be used to synthesize more of the desired compound (FIG. 18). Second, there is an increased likelihood that multiple ligand adducts displayed on biological polypeptide scaffolds will interact with the target (Jencks, W. P., Proc Natl Acad Sci USA (1981) 78:4046-4050); the ligand adducts are combined through polypeptide bonds, instead of by using a single ligand candidate, as is often employed in traditional fragment-based drug discovery methods where ligands bind weakly to the target (Erlanson, D. A., Top Curr Chem (2012) 317:1-32). Third, the multiple ligand adduct structures identified using methods of the claimed invention may provide unique structural information about the relative position of ligand adduct moieties in 3-dimensional space and their biological activity. Such structure activity relationships (SAR) may be derived based on the known principles of protein and peptide secondary structure (Bornscheuer, U. Kazlauskas, R. J. (2011) Curr Protoc Protein Sci, Chapter 26: Unit 26 7). This information speeds the design of hit-to-lead medicinal chemistry optimization efforts.

The library further comprises a population of structurally different ligand adduct moieties at one or more defined positions of a polypeptide's primary amino acid sequence. The library can comprise a population of two (2) or more different and structurally unique ligand adduct moieties. For example, the library can comprise at least 5, 10, 20, 30, 40, 50, 100, 200, 500, 1000, or more different and structurally unique ligand adduct moieties. In one embodiment, the library polypeptides comprise at least 20 structurally different ligand adduct moieties. In another embodiment, the library polypeptides comprise at least 100 structurally different ligand adduct moieties.

The methods described herein for tRNA-ligand adduct libraries may be used to encode polypeptides with site-specific ligand adduct libraries and to select novel polypeptides that have specific target-binding or other activities. Accordingly, provided herein are methods of selecting for a polypeptide (or an mRNA encoding a polypeptide) that interacts with a target or exhibits another desired, specific activity. Also provided herein are methods of using libraries of ligand adduct polypeptides complexes described herein to optimize the binding or functional properties of a polypeptide. A library will generally contain at least 10² members, more preferably at least 10⁶ members, and more preferably at least 10⁹. In some embodiments, the library will include at least 10¹² members or at least 10¹⁴ members. In general, the members will differ from each other; however, it is expected there will be some degree of redundancy in any library.

The library can exist as a single mixture of all members, or can be divided into several pools held in separate containers or wells in multiplex format (i.e. spatially addressable arrays, see, e.g. WO 03046195 as incorporated by reference herein in its entirety), each containing a subset of the library, or the library can be a collection of containers or wells on a plate, each container or well containing just one or a few members of the library, cf. FIG. 18. In some embodiments, the translation product of the encoding nucleic acid can be screened for activity in spatially addressable arrays. An array is a precisely ordered arrangement of elements, allowing them to be displayed and examined in parallel (Emili, A. Q. and Cagney, G. (2000) Nature Biotechnology 18:393-397). It usually comprises a set of individual species of molecules or particles arranged in a regular grid format wherein the array can be used to detect interactions, based on recognition or selection, with a second set of target molecules applied to it. Arrays possess advantages for the handling and investigation of multiple samples. They provide a fixed location for each element such that those scoring positive in an assay are immediately identified, they have the capacity to be comprehensive and of high density, they can be made and screened by high throughput robotic procedures using small volumes of reagents and they allow the comparison of each assay value with the results of many identical assays.

In some embodiments, library members may be compartmentalized in vitro (IVC). (Miller, 0. J., et al., Nat Methods (2006) 3:561-570; Stapleton, J. A. and Swartz, J. R., PLoS One (2010) 5:e15275; Yonezawa, M., et al., Nucleic Acids Res (2003) 31:e118; Tawfik, D. S. and Griffiths, A. D., Nat Biotechnol (1998) 16:652-656).

As a step toward generating the ligand adduct polypeptide libraries of the invention, the mRNA is synthesized. This may be accomplished by direct chemical RNA synthesis or, more commonly, is accomplished by transcribing an appropriate double-stranded DNA template. Such DNA templates may be created by any standard technique (including any technique of recombinant DNA technology, chemical synthesis, or both, cf. United States Patent Application No. 20110172127). In principle, any method that allows production of one or more templates containing a known, random, randomized, or mutagenized sequence may be used for this purpose. In one particular approach, an oligonucleotide (for example, containing random bases) is synthesized to produce a random cassette which is then inserted into the middle of a known protein coding sequence (see, for example, chapter 8.2, Ausubel et aI, Current Protocols in Molecular Biology, John Wiley & Sons and Greene Publishing Company, 1994). To decrease the chances of introducing a premature stop codon, reduced codon sets may used: NNB, NNS, and NNK codons (where N=A/C/G/T, B=C/G/T, S=C/G, K=G/T) are popular choices that still encode all 20 amino acids, but the use of codon sets encoding fewer amino acids may be used as well (Fellouse, F. A., et al., Proc Natl Acad Sci USA (2004) 101:12467-12472). More sophisticated randomization schemes such as MAX results in equal probabilities for all 20 amino acids (or for some predetermined subset thereof), without encoding stop codons.

A diverse library of encoded ligand adduct moieties can be enriched in molecules with the desired properties using in vitro selection methods well known in the art, see, for example FIG. 18. Methods by which members of the library that bind to a target can be enriched include affinity enrichment using immobilized target or binding partner and, for enzymatic activity, affinity to a product of a reaction in which the enzyme has modified itself (with, for example, a mechanism based inhibitor) or a substrate to which it is attached. Furthermore, libraries enriched in target-binding members can be amplified, sequenced, and then subjected to additional design and enrichment cycles. For example, mRNA can be reverse transcribed, producing the cDNAs of the mRNA components. The cDNAs can then be amplified (e.g., by PCR or other amplification methods) and/or sequenced to reveal the structure and sequence of ligand adduct moieties. A preferred method of sample preparation uses digital RNA sequencing Shiroguchi, K., et al., Proc Natl Acad Sci USA (2012) 109:1347-1352. In some embodiments, the PCR product may be mutated with error-prone PCR (Caldwell et al. (1992) PCR Methods Appl. 2:28) or DNA shuffling (Stemmer (1994) Proc. Natl. Acad. Sci. USA 91:10747, Stemmer (1994) Nature 370:389; U.S. Pat. No. 5,811,238; each of which is incorporated herein by reference), before, or after DNA sequencing. New encoded libraries may be designed, gene synthesized, or assembled or amplified and subjected to in vitro transcription, resulting in production of mRNAs that encode the members of the enriched library. In vitro translation of this pool in the presence of the acyl-tRNA analogues of the present invention produces an amplified version of the enriched encoded polymer library. Library members selected and amplified in this way are subjected to further enrichment and amplification, which is repeated as necessary until target members are enriched to the desired extent (e.g., enriched to a level where they are present in sufficient numbers to be detected by binding to a ligand of interest or catalyzing a reaction of interest).

Typically, a purified target (e.g., a protein or any of the target molecules described herein) is conjugated to a solid substrate, such as an agarose or synthetic polymer bead. The conjugated beads are mixed with the display library and incubated under conditions (e.g., temperature, ionic strength, divalent cations, and competing binding molecules) that permit specific members of the library to bind the target. Alternatively, the purified target protein can be free in solution and, after binding to an appropriate polypeptide, the ribosome-mRNA-polypeptide complex or the mRNA-polypeptide complex with a bound target is captured by an antibody that recognizes the target (e.g., target protein) at a site distinct from the site where the displayed polypeptide binds. The antibody itself can be bound to a bead, or it may be subsequently captured by a suitable substrate, such as Protein A or Protein G resins. The binding conditions can be varied in order to change the stringency of the selection. For example, low concentrations of a competitive binding agent can be added to ensure that the selected polypeptides have a relatively higher affinity. Alternatively, the incubation period can be chosen to be very brief, such that only polypeptides with high kₒₙ rates will be isolated. In this manner, the incubation conditions play an important role in determining the properties of the selected ligand adduct polypeptides.

Negative selections against an anti-target can also be employed. In this case, a selection to remove polypeptides with affinity to the substrate to which the target is bound {e.g., Sepharose) is carried out by applying the displayed library to substrate beads lacking the target protein. In some embodiments the library may be precleared by interaction with serum or serum protein anti-targets. This step can remove mRNAs and their encoded polypeptides that are not specific for the target protein. In some embodiments selections for binding may incur in the presence of high cofactor or substrate concentrations in order to preferentially enrich for allosteric ligands. A target can likewise be locked in a catalytically inactive state to facilitate the selection of binders that stabilize the inactive conformation. Numerous references describing how to conduct selection experiments are available. (See, e.g., U.S. Pat. No. 6,258,558; Smith, G. P. and Petrenko, V. A., (1997) Chem. Rev. 97:391-410; Keefe, A. D. and Szostak, J. W. (2001) Nature 15:715-718; Baggio, R. et al. (2002) J. Mol. Recog. 15: 126-134; Sergeeva, A., et al., Adv Drug Deliv Rev (2006) 58:1622-1654).

The frequency at which binding molecules are present in a large library (i.e. >10⁶ members) is expected to be very low. Thus, in the initial selection round, very few ligand adduct polypeptides meeting the selection criteria (and their associated mRNAs) may be expected to be recovered and amplified by RT-PCR. In order to overcome the inherent noise in each round of selection, high-density next-generation sequencing of the PCR product of amplification (FIG. 18, step e) whereby single molecules of mRNA or DNA are amplified and sequenced, can reveal structure-activity relationships regarding the nature of the polymeric chemical structures bound to the target as decoded by sequencing (Buller, F., et al., Bioorganic & Medicinal Chemistry Letters (2010) 20:4188-4192; Larman, H. B., et al., Proc Natl Acad Sci USA (2012) 109:18523-18528).

A preferred DNA sequencing method of the invention is sequencing-by-synthesis approach. The method allows sequencing of a single-stranded DNA by synthesizing the complementary strand along it. Each time a nucleotide (e.g., A, C, G, or T) is incorporated into the growing chain, a cascade of enzymatic reactions is triggered which causes a light signal (see, e.g., Ronaghi et al., 1996, Anal Biochem 242:84-89) or hydrogen ion signal (see, e.g. Rusk N (2011), Nat Meth 8 (1): 44-44)). The technique has been commercialized and further developed by 454 Life Sciences Corp. (Branford, Conn.) to an array-based massively parallel method (see, e.g., U.S. Pat. Nos. 6,956,114 and 7,211,390.), by Life Technologies Corp (Carlsbad, Calif.) Ion Torrent sequencing, and by Pacific Biosciences (Menlo Park, Calif.) using zero-mode wave guides.

Another preferred DNA sequencing method is the Solexa sequencing technology commercially available from Illumina Inc. (San Diego, Calif.), which is based on massively parallel sequencing of millions of fragments using clonal single molecule array technology and novel reversible terminator-based sequencing chemistry. This approach relies on attachment of randomly fragmented DNA to a planar, optically transparent surface and solid phase amplification to create an ultra-high density sequencing flow cell with >10 million clusters, each containing approximately 1000 copies of template per sq. cm. These templates are sequenced using a robust four-color DNA sequencing-by-synthesis technology that employs reversible terminators with removable fluorescence. This approach ensures high accuracy and avoidance of artifacts with homopolymeric repeats. High sensitivity fluorescence detection is achieved using laser excitation and total internal reflection optics. Short sequence reads are aligned against a reference genome and genetic differences are called using a specially developed data pipeline. Alternative sample preparation methods allow the same system to be used for a range of other genetic analysis applications, including gene expression. See, e.g., U.S. Pat. Nos. 6,787,308, 6,833,246, 6,897,023, 7,057,026, 7,115,400, and 7,232,656 and United States Patent Application 20030022207, 20030064398, 20040106110, and 20060188901 for a description of the Solexa sequencing technology and related embodiments. Overall, the Illumina platform is most suitable because of its combination of relatively low base-calling error rates and relatively low cost. Other preferred sequencing methods include DNA sequencing using nanopores or DNA sequencing by hybridization.

Statistical analysis of the vast number of DNA sequences can be performed to identify desired ligand adduct moiety structures for subsequent rounds of affinity-based selection. Ligand adduct moieties with the desired activity, such as, e.g., those which increase the binding affinity, are enriched in the selected population, while variants with undesired activities will be depleted in the selected population, see FIG. 18. The extent to which a ligand adduct moiety increases or decreases its binding will be reflected in the extent to which that a ligand adduct moiety is enriched or depleted in the population. Such analysis can include computer analysis of the raw DNA sequences. In one embodiment, the raw DNA sequences can be aligned and compared with the reference sequence to identify the ligand adduct moiety. The frequency of each sequence observed at each position can be tabulated for three categories (increase, decrease, or neutral) and compared with the reference. In a preferred embodiment, the sequence distribution of the library before selection is fit to negative binomial distribution density function. A quantile-quantile plot of the experimental and theoretical distributions shows equal distribution of all library members in the library before selection, whereas deviations from the diagonal indicate enrichment of specific binders after selection. In some embodiments, DNA sequence data is filtered to eliminate singleton sequences.

Preferably, the sequence information may be analyzed using a computer application which can translate the sequence information into e.g. encoded ligand adduct structures. A computer application may preferably be used to analyze such encoded structures include quantitative and qualitative structure-activity relationship (SAR) analyses e.g. such as analyzing and/or clustering structural fingerprints common to enriched encoded structures. This approach can be refined by initially identifying the members of the library by methods of structure-based or nonstructure based computer drug-modeling. Suitable non-structure based methods are disclosed in e.g. U.S. Pat. Nos. 5,307,287, 5,025,388 (a method known as COMFA). An alternative is HASL (Hypothetical Active Site Lattice; Hypothesis Software). Both these methods are based on 3D-QSAR. A feasible structure-based approach is Rosetta design methodology.

### Target Identification and Validation

In another embodiment of the present invention, targets may be isolated or identified that are involved in pathological processes or other biological events. In this aspect, the target molecules are again preferably proteins or nucleic acids, but can also include, among others, carbohydrates and various molecules to which specific molecule ligand binding can be achieved. In principal, the technology could be used to select for specific epitopes on antigens found on cells, tissues or in vivo. These epitopes might belong to a target that is involved in important biological events. In addition, these epitopes might also be involved in the biological function of the target. In some embodiments the technique may be used to characterize the specificity of chemical entities that interact with DNA or genome-associated proteins as determined by genome sequencing.

Phage display with antibodies and peptide libraries has been used numerous times successfully in identifying new cellular antigens, Sergeeva, A., et al., Adv Drug Deliv Rev (2006) 58:1622-1654; Arap, W., et al., Science (1998) 279:377-380; Pasqualini, R. and Ruoslahti, E., Nature (1996) 380:364-366). Especially effective has been selection directly on cells suspected to express cell-specific antigens. Importantly, when selecting for cell-surface antigen, the amplifiable molecule can be maintained outside the cell. This will increase the probability that the ribosome-displayed molecules will be intact after release for the cell surface. In some embodiments cells may be lysed to release intracellular targets for genome-wide localization of small molecules, such as chromatin-associated targets.

In vivo selection of ribosome-displayed molecules has tremendous potential. By selecting from libraries of ribosome-displayed molecules in vivo it is possible to isolate molecules capable of homing specifically to normal tissues and other pathological tissues (e.g. tumors). This principle has been illustrated using phage display of peptide libraries (Pasqualini, R. and Ruoslahti, E., Nature (1996) 380:364-366). This system has also been used in humans to identify antibody epitopes that localized to tumors (Shukla, G. S., et al., Cancer Immunol Immunother (2013) 62:1397-1410). A similar selection procedure could be used for the ribosome-encoded chemical libraries of the present invention. The coding DNA sequence in phage display is protected effectively by the phage particle allows selection in vivo. Accordingly, the stability of the message in vivo will be important for amplification and identification. The messenger RNA can be stabilized against degradation using various modified RNA derivatives encoding the displayed molecule (Kariko, K., et al., Mol Ther (2008) 16:1833-1840). Other types of protection are also possible where the message is shielded for the solution using various methods. This could include for example liposomes or other sorts of protection.

### EXAMPLES

The following examples are provided by way of illustration only and not by way of limitation. Those of skill will readily recognize a variety of noncritical parameters which could be changed or modified to yield essentially similar results.

### Example 1

### Preparation of tRNA with a Highly Pure CCA 3′-Hydroxyl

This example illustrates a method for in vitro transcription of a cis-acting ribozyme fusion (Avis, J. M., Conn, G. L., & Walker, S. C. in Recombinant and in Vitro RNA synthesis: Methods and Protocols, Methods in Molecular Biology, vol. 941, pp. 83-98, (2012)) for the production of an optimized 75 nucleotide Methanococcus jannaschii (Mj) tRNA_{CUA}^{Tyr} (Young, T. S., et al., J Mol Biol (2010) 395:361-374; Albayrak, C. and Swartz, J. R., Nucleic Acids Research (2013) 41:5949-5963) with a highly pure CCA 3′-hydroxyl, using the DNA template illustrated in FIG. 4. Optimized in vitro transcription reactions were performed in 20 mL glass scintillation vials containing 120 mM HEPES (pH 7.5), 20 mM NaCl, 30 mM MgCl₂, 30 mM DTT, 2 mM spermidine, 0.011 μg/mL S. cerevisiae pyrophosphatase; 4 mM each of ATP, CTP, UTP, GTP at pH 7, 0.008 mg/ml T7 RNA polymerase, and 0.03 μg/mL of pGB014 DNA template plasmid. Transcription reactions were incubated for 5 hr at 37° C. Autocatalytic cleavage by the HDV ribozyme left a 2′, 3′ cyclic phosphate at the tRNA 3′ termini (FIG. 5B; Been, M. O. and Wickham, G. S., Eur. J. Biochem (1997), 247:741-753; Handbook of RNA Biochemistry, Volume 1, edited by R. K. Hartmann, A. Bindereif, A. Schon, and E. Westhof, Wiley-VCH Verlag GmbH & Co. Weinheim, FRG (2005)).

The tRNA containing transcription mixture was refolded on a heating block at 70° C. for 30 min., then removed and slowly cooled to room temperature suspended in buffer A (50 mM Bis-Tris pH 6.2, 0.5 mM EDTA), followed by filtration through a 0.45 ium filter. The sample was then loaded onto a strong anion exchange column (3 mL Fractogel TMAE; EMD Chemicals, Gibbstown, N.J.) pre-equilibrated with Buffer A using an ÄKTA Explorer 10 chromatography system (GE Healthcare Life Sciences, Piscataway, N.J.), controlled using Unicorn ver. 5.4 software. The column was washed with 7.5% high ionic strength buffer B (50 mM Bis-Tris, pH6.2, 0.5 mM EDTA, 2 M NaCl) until the UV detector returned to the equilibration baseline. tRNA was eluted with a 10 column volume gradient from 15%-50% buffer B, as shown in FIG. 6A. tRNA_{CUA} containing fractions were ethanol precipitated by addition of 3M Sodium Acetate pH 5.5 at a volume of 1:10 and adding ethanol at 2 times the volume of the reaction. The ethanol precipitated reaction was then left overnight at −20° C., or for 30 min at −80° C. The precipitated nucleic acids were pelleted by centrifugation at 20,000×g for 30 min at 4° C. After the liquid was carefully decanted, the pellet was dried for 10 min. at room temperature, and subsequently stored in MilliQ water. tRNA-containing fractions were confirmed by running 1 μL samples diluted in 9 μL water mixed 1:1 with 10 μL RNA Gel Loading Buffer (95% deionized formamide, 0.025% (w/v) bromophenol blue, 0.025% (w/v) xylene cyanol FF, 5 mM EDTA, 0.025% (w/v) SDS) on 10% TBE/Urea PAGE (polyacrylamide gel electrophoresis) precast gels (Bio-RAD, Hercules, Calif.) stained with RNA Stain (0.5 M Sodium Acetate pH 6.1, 0.05% (w/v) methlyene blue). tRNA_{CUA}^{Tyr} cyclic 3′ phosphate containing fractions were pooled as shown in FIG. 6B. A260 and A260/A280 measurements were taken using a Nanodrop 2000c UV spectrophotometer (Thermo Fisher, Hudson, N.H.) to determine the concentration of the Mj tRNA_{CUA}^{Tyr} cyclic 3′ phosphate (1 A260=40 ng/uL RNA) and purity (typically A260/A280≥2.0).

### Example 2

### Preparation of tRNA-pGB028

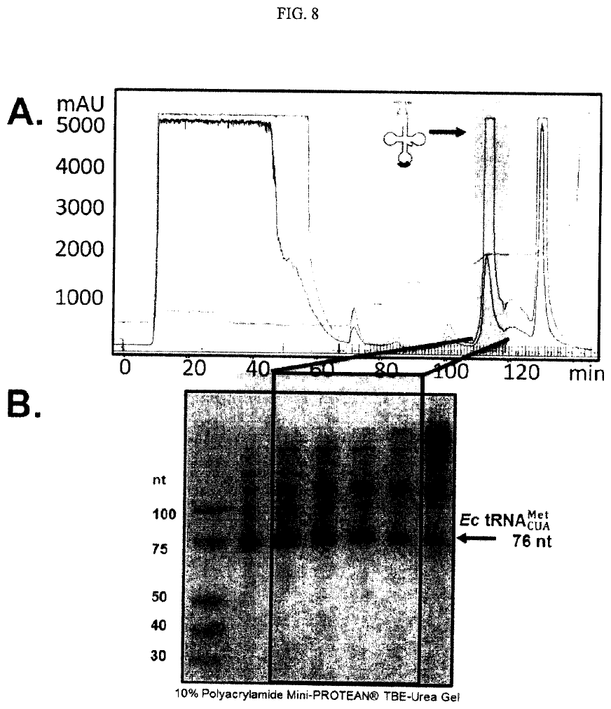

tRNA transcribed from DNA plasmid pGB028 (FIG. 7) and refolded at 70° C. for 30 min was purified essentially the same as in Example 1. The transcription yields were significantly higher, but the purified tRNA was contaminated by a significant fraction of higher MW RNA (presumably HDV ribozyme, cf. FIG. 8).

### Example 3

### Preparation of Mj TyrRS Enzyme Variants

This example illustrates the preparation of an engineered aminoacyl tRNA synthetase (aaRS) enzyme corresponding to a polyspecific aaRS enzyme from Methanococcus jannaschii (Mj) pCNF TyrRS described by Young, D. D., et al., Biochemistry (2011) 50:1894-1900 that may be used to charge tRNAs with non-canonical amino acids containing phenylalanine side chains substituted with reactive moieties. The T7-based plasmid pGB008, coding for pCNPhe Mj Tyrosyl RS with a C-terminal 6× His tag, was used to transform E. coli strain BL21 (DE3) and grown to an OD₆₀₀ of 0.5-0.6 in 2 L of 2× Yeast Extract Tryptone medium (2×YT) divided into 3 Tunair flasks. Isopropyl-β-D-thiogalactoside (IPTG) was added to a final concentration of 1 mM and the cells were grown for additional 4-5 h at 37° C. Cells were harvested at 5,000×g for 15 min at 4° C. The cell pellet was washed by suspending it in 20 mL of lysis/equilibration buffer (300 mM NaCl, 10 mM imidazole, 50 mM K₂HPO₄/KH₂PO₄, pH 8.0) and frozen at −80° C. overnight. The cell pellets were thawed and resuspended in 2-5 ml/g cell pellet (about 40 mL) lysis buffer total. DNAse I (1 U), phenyl methylsulfonyl fluoride (PMSF) to a final concentration of 100 μM, and 1 mg/mL of lysozyme were added to the cell suspension at 4° C. The cells were then lysed by sonication with a 2 mm probe using a Vibra-Cell Ultrasonic Liquid Processor, Model VC-505 (Sonics & Materials, Inc. Newton, Conn.) for 12 cycles of 30 sec pulses followed by 1 min on ice in between each cycle. All sonication was conducted in a cold room set at 4° C. The lysate was then centrifuged at 14,000×g at 4° C. for 30 min. The supernatant was sterile filtered using a 0.45 μm pore size filter for AKTA Explorer 10 FPLCpurification.

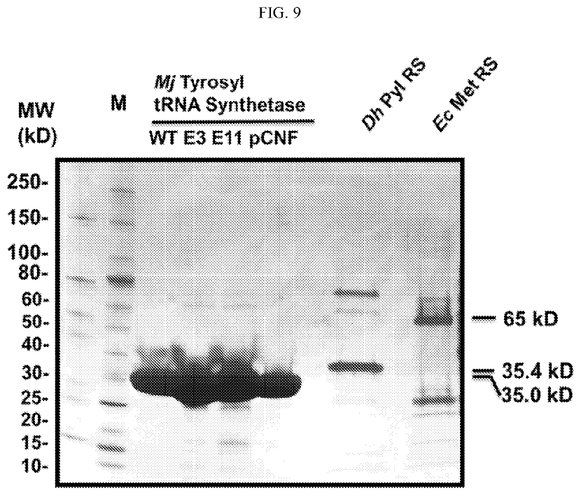

The 6×His-tagged tRNA synthetase enzymes were purified on an AKTA Explorer 10 FPLC, using a 1 mL HisTrap HP Column (GE Lifesciences) preequilibrated with lysis/equilibration buffer A (300 mM NaCl, 10 mM imidazole, 50 mM K₂HPO₄/KH₂PO₄, pH 8.0). After loading, the 6×His tagged protein was eluted using a 20 column volume gradient from 2-100% buffer B (300 mM NaCl, 500 mM imidazole, 50 mM K₂HPO₄/KH₂PO₄, pH 8.0). Fractions were collected and confirmed by running 10 μL of sample mixed 1:1 with 2× Tricine loading buffer on a 12% Tris-Glycine SDS PAGE (Life Technologies). Protein containing fractions were then concentrated using Slidelyzer 10 kDa MWCO cassettes (Pierce), then buffer exchanged 3 times with 10× concentration each time or dialyzed at 4° C. for 12 hrs against PBS buffer (10 mM Na₂HPO₄, 2 mM KH₂PO₄, pH 7.4, 2.7 mM KCl and 137 mM NaCl) with, 10% Glycerol. The enzymes were typically >90% pure by SDS-PAGE analysis (FIG. 9).

### Example 4

### Preparation of Aminoacyl tRNA (p-AzidoPhe-tRNA_{CUA}^{Tyr})

Aminoacylation of Mj tRNA_{CUA}^{Tyr} cyclic 3′ phosphate with p-azido-L-phenylalanine (pAzPhe; Chem-Impex International, Wood Dale, Ill.), schematically illustrated in FIG. 5C & 5D, was performed in 50 mM HEPES pH 7.5, 40 mM KCl, and 10 mM MgCl₂ with 2 mM ATP pH 7.1, 0.1% Triton X-100, 5 mM pAzF (X. 252 nm; c=16,000; Schwyzer, R. and Caviezel, M., Helvetica Chimica Acta (1971) 54:1395-1400) from a stock solution dissolved in 100% DMSO, 25 μM Mj pCNF TyrRS enzyme, 2 U/ml PPiase (Roche Diagnostics, Indianapolis, Ind.), 0.2 U/μl phage T4 polynucleotide kinase (PNK; Thermo Fisher), and 25 μM Mj tRNA_{CUA}^{Tyr} cyclic 3′ phosphate, purified as shown in FIG. 6C. The reaction was incubated for 30 min at 37° C. without TyrRS or pAzPhe to allow time for the removal of the 2′-3′ cyclic phosphate from tRNA by PNK, as previously described (Handbook of RNA Biochemistry, Volume 1, edited by R. K. Hartmann, A. Bindereif, A. Schon, and E. Westhof, Wiley-VCH Verlag GmbH & Co. Weinheim, FRG (2005), pp 33.). The TyrRS and pAzPhe were then added and the reaction proceeded for 30 additional min at 37° C.

The aminoacylation reaction was quenched with 1/10 vol. ice cold 3 M NaOAc pH 5.5 and immediately extracted with one vol. 25:24:1 phenol:chloroform:isoamyl alcohol pH 5.2 (Fisher), mixed for 2 min, and centrifuged for 20 min at 14,000× g at 4° C. The aqueous layer was applied to a Bio-Spin 6 spin column (Bio-Rad, Hercules, Calif.) pre-equilibrated in 0.3 M NaOAc pH 5.5 and centrifuged for 4 min at 1,000× g to remove excess ATP and pAzPhe. The flow-through was precipitated in 2.5 vol. 95% EtOH, incubated for 20 min at −80° C., and centrifuged at 14,000×g at 4° C. for 30 min. The pelleted aminoacyl pAzPhe-tRNA_{CUA}^{Tyr} was resuspended in 50 mM KPi pH 5.

### Example 5

### Preparation of aminoacyl tRNA p-AcetylPhe-tRNA_{CUA}^{Tyr})

Aminoacylation of Mj tRNA_{CUA}^{Tyr} cyclic 3′ phosphate with p-acetyl-L-phenylalanine (pAcPhe; Combi-Blocks, Inc., San Diego, Calif.) was performed in 50 mM HEPES pH 7.5, 40 mM KCl, and 10 mM MgCl₂ with 2 mM ATP pH 7.1, 0.1% Triton X-100, 5 mM pAcPhe, from a stock solution dissolved in DMSO, 25 μM Mj pCNF TyrRS enzyme, 2 U/ml PPiase (Roche Diagnostics, Indianapolis, Ind.), 0.2 U/μl phage T4 polynucleotide kinase (PNK; Thermo Fisher), and 25 μM Mj tRNA_{CUA}^{Tyr} cyclic 3′ phosphate, purified as shown in FIG. 6. The reaction was incubated for 30 min at 37° C. without pCNF TyrRS enzyme or pAcPhe. The pCNF TyrRS enzyme and pAcPhe were then added and the reaction proceeded for an additional 30 min at 37° C.

The aminoacylation reaction was quenched with 1/10 vol. ice cold 3 M NaOAc pH 5.5 and immediately extracted with one vol. 25:24:1 phenol:chloroform:isoamyl alcohol pH 5.2 (Fisher), vortexed for 2 min, then centrifuged for 20 min at 14,000× g at 4° C. The aqueous layer was applied to a Bio-Spin 6 spin column (Bio-Rad, Hercules, Calif.) pre-equilibrated in 0.3 M NaOAc pH 5.5 and centrifuged for 4 min at 1,000× g to remove excess ATP and pAcPhe. The flow-through was precipitated in 2.5 vol. 95% EtOH, incubated for 20 min at −80° C., and centrifuged at 14,000×g at 4° C. for 30 min. The pelleted tRNA and aminoacyl pAcPhe-tRNA_{CUA}^{Tyr} (86%) were resuspended in 50 mM KPi pH 5.

### Example 6

### Assaying a Library of Aminoacyl-tRNA Using HIC-HPLC

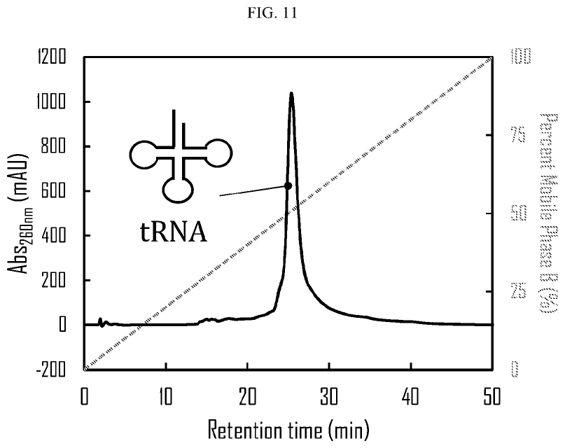

This example describes a method for determining the extent of aminoacylation of tRNA with non-canonical amino acid ligand adducts. Evaluation of aminoacyl-tRNA_{CUA}^{Tyr} or -tRNA_{CUA}^{Met} is accomplished by hydrophobic interaction chromatography (HIC) using C5 HIC-HPLC resolution of the aminoacylated and unaminoacylated moieties of tRNA as illustrated in FIG. 11 and FIG. 12. This method monitors the extent of aminoacylation of tRNA after it has been processed and is ready to be used for in vitro translation into proteins. A rapid, non-radioactive assay enables the direct monitoring of each batch of acyl-tRNA ligand adduct produced and allows for rigorous quality control of the process, including extent of ligand adduct formation.

Using an HP/Agilent 1050 HPLC system with a multiple wavelength detector and ChemStation Rev. A.10.02 software, a C5 2.0 mm×250 mm column (Jupiter, 5 μm pore size; Phenomenex) was equilibrated in high salt buffer A (50 mM potassium phosphate, 1.5 M ammonium sulfate, pH 5.7). acyl-tRNA non-canonical amino acid ligand adducts (1-50 μg) are mixed with 50 μl of buffer A, and then injected on the column with a gradient from buffer A to buffer B (50 mM potassium phosphate, pH 5.7 and 5% isopropanol) over 50 min. The resulting chromatograms were typically monitored at 214 and 260 nm. The relative amounts of tRNA, aminoacyl-tRNA, and/or aminoacyl-tRNA ligand adduct were determined by peak height and/or integrated area.

### Example 7

### Assaying a Library of Aminoacyl-tRNA Using Capillary Electrophoresis

Resolution between aminoacyl-tRNA^{Phe}_{CUA} charged with ligand adduct moieties linked to a non-natural amino acid side chain, aminoacyl-tRNA^{Phe}_{CUA} charged with a non-natural amino acid side chain, and intact tRNA^{Phe}_{CUA} is accomplished by capillary electrophoresis (FIG. 13) based on the differences in molecular weight/charge using an Agilent G1600 CE system with an untreated fused-silica capillary (50 μm×72 cm) equilibrated in 25 mM phosphate buffer pH 7. Pelleted samples of ethanol-precipitated aminoacy-tRNA at about 50 ng/uL are analyzed electrophoretically at 30 kV. The resulting electropherograms are monitored at 260 nm and integrated to determine the fractions of aminoacylated and unaminoacylated tRNA. In cases where secondary structure of these molecules interferes and broadens the observed peaks, 10% formamide or 3 M urea in the 25 mM phosphate buffer are added to the running buffer.

### Example 8

### Kinetics of Aminoacyl-tRNA Formation (Tyr-tRNA_{CUA}^{Tyr})

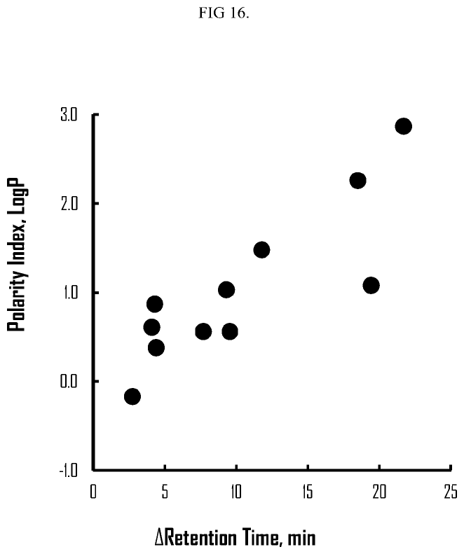

The kinetics of aminoacyl-tRNA formation were monitored by hydrophobic interaction chromatography (HIC-HPLC) using an HP/Agilent 1050 HPLC system with a multiple wavelength detector and ChemStation Rev. A.10.02 software. A C5 2.0 mm×250 mm column (Jupiter, 5 μm pore size; Phenomenex) was equilibrated in high salt buffer A (50 mM potassium phosphate, 1.5 M ammonium sulfate, pH 5.7). Aminoacyl-tRNA samples prepared at various time points, essentially as described in Example 4, but with Tyrosine, instead of pAzPhe, were quenched with 1/10^{th} volume 3M Acetic Acid, pH 5.9, and purified by Bio-Spin column treatment. Time point samples were mixed with 50 μl of buffer A (50 mM potassium phosphate, 1.5 M ammonium sulfate, pH 5.7) and then injected on the column with a gradient from buffer A to buffer B (50 mM potassium phosphate, pH 5.7 and 5% isopropanol) over 50 min. The resulting chromatograms were typically monitored at 214 and 260 nm. The relative amounts of tRNA, aminoacyl-tRNA, and/or aminoacyl-tRNA ligand adduct were determined by peak height and/or integrated are shown in FIG. 14. The relative change in retention time between aminoacyl-tRNAs and tRNA 2,3′-cyclic phosphate (FIG. 15) was directly related to hydrophobicity (estimated by the parameter Log P) of the ncAA as shown in FIG. 16.

### Example 9

### Preparation of an Acyl-tRNA Non-Canonical Amino Acid Ligand Adduct (Click Chemistry)

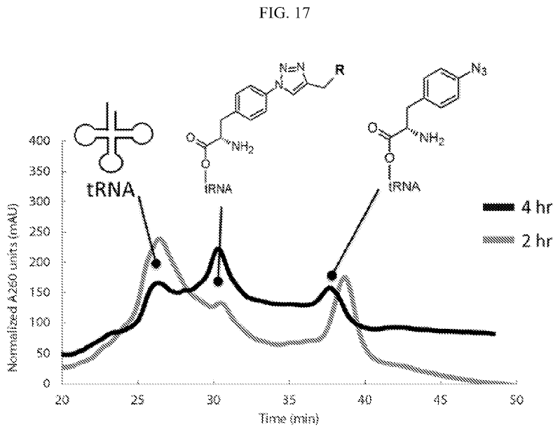

To 30 μg of pAzPhe-tRNA_{CUA}^{Tyr}, prepared as described in Example 4, was added 0.5 mM 3,3′,3″-(4,4′,4″-(Nitrilotris(methylene))tris(1H-1,2,3-triazole-4,1-diyl))tris(propan-1-ol) (THPTA) ligand, 100 μM CuCl₂, 500 mM NaOAc, pH 5.5, 2 mM propargyl alcohol, and 5 mM ascorbic acid titrated to pH 5.3. The coupling reaction was started with the addition of ascorbate or alkyne and allowed to proceed for several hours at 30° C. The reaction mixture was then applied to a Bio-Spin 6 spin column (Bio-Rad) and centrifuged for 4 min at 1,000× g in order to remove reagents less than 6,000 MW in size. HIC-HPLC analysis (FIG. 17 and FIG. 18) showed formation of a click-adduct that is subsequently used in cell-free protein synthesis.

### Example 10

### Preparation of an Acyl-tRNA Non-Canonical Amino Acid Ligand Adducts (Oxime Ligation)

To 15 μg of p-AcetylPhe-tRNA_{CUA}^{Tyr}, prepared as described in Example 6, was added 50 mM hydroxylamine or methoxyamine in 50 mM KPi, pH 5.7. The coupling reaction was allowed to proceed for two hours at 30° C. The reaction mixture was then applied to a Bio-Spin 6 spin column (Bio-Rad) and centrifuged for 4 min at 1,000× g in order to remove reagents less than 6,000 MW in size. The extent of oxime ligation was >95% as determined by HIC-HPLC analysis (FIG. 19).

Thus aminoacyl-tRNA ligand adducts of the present invention can be produced by a reaction that is surprisingly efficient and simple. In general, even inefficiently (<20%) aminoacylated nnAA analogues may be used to form aminoacyl-tRNA ligand adducts. Optimization of successful candidates and aminoacyl-tRNA synthetase (RS) engineering allow structurally similar analogs to be used (see, for example, FIG. 20 which shows amino acid side chain polarity on the x-axis and size on the y-axis for aminoacyl-tRNA structures capable of ribosome-directed translation such as the 20 canonical amino acids, indicated by the 1-letter amino acid code, (ii) representative literature examples of non-canonical amino acids incorporated into proteins and (iii) representative examples of triazole ligand adducts.

### Example 11

### Assaying Acyl-tRNA and Ligand Adduct Acyl-tRNA Using ³²P

This example describes representative methods for assaying aminoacyl-tRNAs charged with non-canonical amino acids, as well as for monitoring formation of acyl-tRNAs with non-canonical amino acid ligand adduct reactions, using radioactivity.

[³²P] A76-End Labeled tRNA.

tRNA, produced as in Example 1, is end labeled at the 3′ A76 with [³²P] by CCA enzyme catalyzed exchange with α-[³²P]-AMP, using modifications of previously described procedures (Francklyn, C. S., et al., Methods (2008) 44:100-118). tRNA produced as in Example 1 (9 μL of 100 μM tRNA_{CUA}^{Tyr}) is incubated with tRNA nucleotidyl transferase in 50 mM glycine, pH 9.0, 10 mM MgCl₂, 0.3 μM α-³²P-ATP (Perkin Elmer, Waltham, Mass.), 0.05 mM PPi for 5 min at 37° C. One (1) μ1 of 10 μM CTP and 10 U/ml (1 Unit) of inorganic pyrophosphatase (Sigma Aldrich, St. Louis, Mo.) is then added for an additional 2 min. The reaction is quenched with 1/10^{th} volume of 3 M NaOAc, pH 5.2. The aqueous phase is applied to a Bio-spin P6 spin column (Bio-Rad, Hercules, Calif.) 2× to remove unincorporated α-³²P-ATP. The ³²P-end labeled tRNA is diluted 1:5 with water and refolded by heating to 70° C., prior to use.

[³²P] Radioactive Assay to Monitor Aminoacylation of tRNA.

³²P-end labeled tRNA_{B4}^{Sep} aminoacylated with 10 mM phospho-Threonine in 50 mM HEPES pH 8.1, 40 mM KCl, 75 mM MgCl₂, 5 mM ATP, 10 uM tRNA_{B4}^{Sep}, 10 mM DTT, and 1-100 μM aminoacyl-tRNA synthetase SepRS for 30 min at 37° C. The products of the aminoacylation reaction are digested with P1 nuclease for 20-60 min at room temperature, and 1 μl aliquots are spotted on a pre-washed (water) PEI cellulose TLC plates and allowed to air dry. [³²P]-AMP is resolved from non-canonical aminoacyl-³²P-AMP in 5% acetic acid and 100 mM ammonium chloride. The plates are imaged by phosphoimaging, and the relative areas used to determine the extent of phosphothreonine aminoacylation of tRNA_{B4}^{Sep}.

[³²P] Assay to Monitor Acy-tRNA Ligand Adduct Formation.

³²P-end labeled pAzPhe-tRNA_{CUA}^{Tyr} produced from the above procedure is reacted with propargyl alcohol in a Cu(I)-catalyzed “click chemistry” reaction as described above. At various time points, aliquots are removed and digested with P1 nuclease for 20-60 min at room temperature; 1 μl aliquots were spotted on a pre-washed (water) PEI cellulose TLC plates and allowed to air dry. [³²P]-AMP is resolved from either non-canonical amino acyl ³²P-AMP or ligand adduct non-canonical amino acyl-³²P-AMP in 5% acetic acid and 100 mM ammonium chloride. The plates are imaged by phosphoimaging, and the relative areas used to determine the extent of ligand adduct formation.

### Example 12

### Engineering Ef-Tu for Phospho-Threonine (p-Thr) Translation

This example illustrates methods for engineering Ef-Tu variants for efficient ribosome-directed translation of non-canonical amino acid ligand adducts. An alanine scan of the mutations found in EF-Sep by Lee et al. Lee, S., et al., Angew Chem Int Ed Engl (2013) 52:5771-5775 can be used to identify variants that are expected to modulate the binding of p-Thr-tRNA, e.g. (FIG. 21). DNA templates coding for EF-Tu variant expression, with an N-terminal His-tag, are assembled by overlap PCR with appropriate oligos and arrayed as transcription/translation reactions in 96-well microtiter plates, see for example Yin, G., et al., MAbs (2012) 4:219-227. The p-Thr-tRNA is produced using threonine, L-[¹⁴C(U)] phosphate (American Radiolabeled Chemicals). The variants were purified by IMAC, then incubated with [¹⁴C(U)] p-Thr-tRNA in EF-Tu-pGTP protection assays (Sanderson, L. E. and Uhlenbeck, O. C., Biochemistry (2007) 46:6194-6200; Park, H.-S., et al., Science (2011) 333:1151-1154) to identify Ef-Tu variants that are competent for ribosome-directed translation of acyl-tRNAs with non-canonical amino acid ligand adducts.

### Example 13

### Preparation of an E. coli Cell-Free Lysate for Transcription/Translation

Genome Engineered Strains. A major limitation of UAG encoded nnAAs is that the ribosomal incorporation (‘suppression’) efficiency and therefore protein yield is lowered (c.f. FIG. 6D), due to competition with Release Factor 1 (RF1) that terminates translation at the UAG codon Harris, D. C. and Jewett, M. C., Curr Opin Biotechnol (2012) 23:672-678; Hoesl, M. G. and Budisa, N., Current Opinion in Biotechnology (2012) 23:751-757. We designed a genome-encoded RF1 variant (ΔprfA) that is functional in the cell during fermentation but is inactive during cell-free protein synthesis prepared according to WO2014058830, incorporated herein in its entirety. Similarly, P1 phage lysates from the single gene knockout strains ΔserB784 kan and ΔphoA748(del)::kan (Coli Genetic Stock Center) were transduced sequentially into a ΔprfA mutated strain. Kanamycin resistant colonies were isolated after each transduction and the kan gene eliminated with a curable helper plasmid that encodes the FLP recombinase Datsenko, K. A. and Wanner, B. L., Proc Natl Acad Sci USA (2000) 97:6640-6645. The ΔserB, ΔphoA, and ΔAmpC strain genotypes are confirmed by colony PCR. The final ΔserB ΔphoA ΔprfA and ΔAmpC strains were tested for growth rate (FIG. 22) and used to make cell-free extracts.

Cell Culture.

E. coli strains were cultured overnight, diluted 1:100 into 500 mL of warm 2× yeast extract and tryptone (YT) growth media, and grown at 37° C. with 250 rpm agitation in a 2.5 L baffled tunair flask (Sigma Aldrich, St. Louis, Mo.). The cell culture was inoculated with 1 mM isopropyl β-D-1-thiogalactopyranoside (IPTG) at an optical density (OD₆₀₀) of 0.6, and the agitation was increased to 280 rpm. Cells were harvested at mid to late logarithmic growth phase (ca. 1.5 OD₆₀₀) by centrifugation at 8000×g in a SLA-3000 rotor (Thermo Scientific) at 4° C. for 30 min. The supernatant was removed, and cell pellets were washed with greater than 25 mL ice-cold buffer A (10 mM Tris acetate (pH 8.2), 14 mM magnesium acetate, and 60 mM potassium glutamate) per gram of wet cell pellet and centrifuged at 3,000×g at 4° C. for 12 min. The wash step was repeated, and the final cell pellet was weighed. Cell pellets that were not lysed the same day were flash-frozen in liquid nitrogen and stored at −80° C. prior to lysis.

Cell Lysis and Lysate Preparation.

Cell pellets were suspended in 1 mL of ice-cold buffer A per gram of pellet. Cell suspensions were lysed using a Vibra-Cell VC 505 probe sonicator (Sonics & Materials, Inc., Newtown, Conn.) with a 2 mm microtip at a frequency of 20 kHz. The sonication was performed in a cold-room, and the sample was kept on ice to prevent heating in the sample during sonication. For cell suspension volumes up to 2 mL (in a 2 mL microcentrifuge tube), sonication was performed by using six cycles of 30 sec sonication and 60 sec cooling intervals on ice. For 2-5 mL suspensions (in a 15 mL Falcon tube), 48 cycles of 30 sec sonication and 60 sec cooling intervals were performed, with a maximum cell lysis efficiency observed at 30 cycles. Following lysis, the extract was centrifuged for 35 min at 12,000× g at 4° C. The supernatant was transferred to a new tube and incubated with shaking at 37° C. for 30 min. The final cell-free lysate was prepared by centrifuging at 14,000× g at 4° C. and keeping the supernatant. The extract was either used immediately or divided into aliquots, flash-frozen in liquid nitrogen, and stored at −80° C. until use.

### Example 14

### Determination of Lysate Activity

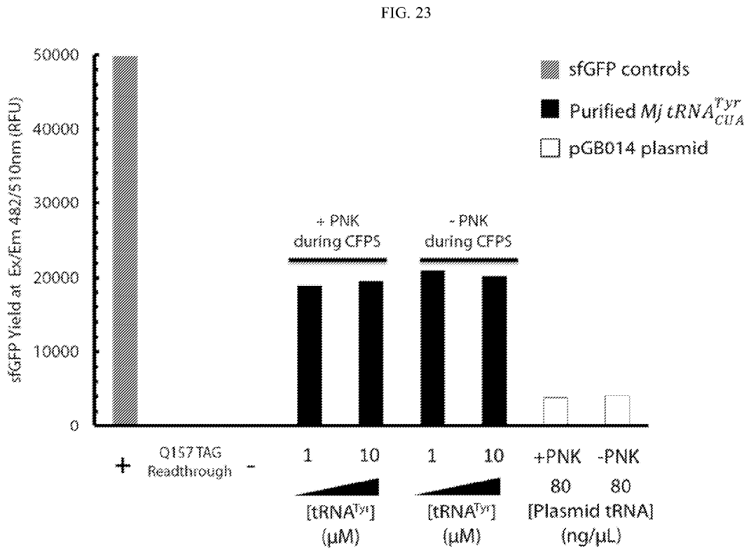

Cell lysate activity was determined as described by Shrestha and co-workers Shrestha, P., et al., Biotechniques (2012) 53:163-174 with the following modifications. Per 20 μL cell-free protein synthesis reaction, 5 μL of lysate, 5 μL of 4× cell-free pre-mix Yang, W. C., et al., Biotechnology progress (2011), and 12 μg/mL of a T7-polymerase based plasmid coding for superfolder GFP were used. For UAG suppression assays, the corresponding Q157TAG GFP coding plasmid was used. The cell-free transcription-translation reactions were incubated at 30° C. for 16 h in a sealed Corning flat bottomed 384-well black or Corning half-area 96-well clear bottom microtiter plates (Corning, Inc., Corning, N.Y.), and relative fluorescence units (RFU; excitation wavelength at 482 nm and emission wavelength at 510 nm) were read using a SpectraMax M2 plate reader (Molecular Devices, Sunnyvale, Calif.). See FIG. 23.

### Example 15

### Cell-Free Transcription/Translation Reaction

This example describes conditions for cell-free transcription/translation reactions. Reactions were run at 30° C. containing 8 mM magnesium glutamate, 10 mM ammonium glutamate, 130 mM potassium glutamate, 35 mM sodium pyruvate, 1.2 mM AMP, 0.86 mM each of GMP, UMP, & CMP, 2 mM amino acids (1 mM for tyrosine), 4 mM sodium oxalate, 1 mM putrescine, 1.5 mM spermidine, 15 mM potassium phosphate, 100 nM T7 RNA polymerase, 2-50 nM DNA template(s), cell-free lysate prepared according to Shrestha, P., et al., Biotechniques (2012) 53:163-174. tRNA was added to a concentration of 1-50 μM (FIG. 23) in the presence of 25 μM RS enzymes. For reactions containing linear DNA templates, 5 μM λ-GamS protein is added in order to inhibit recBCD catalyzed nuclease activity. Sitaraman, K., et al., J Biotechnol (2004) 110:257-263.

### Example 16

### Cell-Free Transcription/Translation Reaction with Acyl-tRNA Ligand Adduct Moiety

Reactions were run as in example 15 except that 1-10 uM p-Azido Phenylalanine aminoacyl tRNA_{CUA}^{Tyr} was added without the addition of RS enzymes (FIG. 24). In the absence of the recycling enzyme, addition of tRNA_{CUA}^{Tyr} alone, gives no suppression (protein synthesis) as measured by sf GFP fluorescence.

### Example 17

### High-Throughput Cell-Free Expression of a Polypeptide Library of Ligand Adduct Moieties

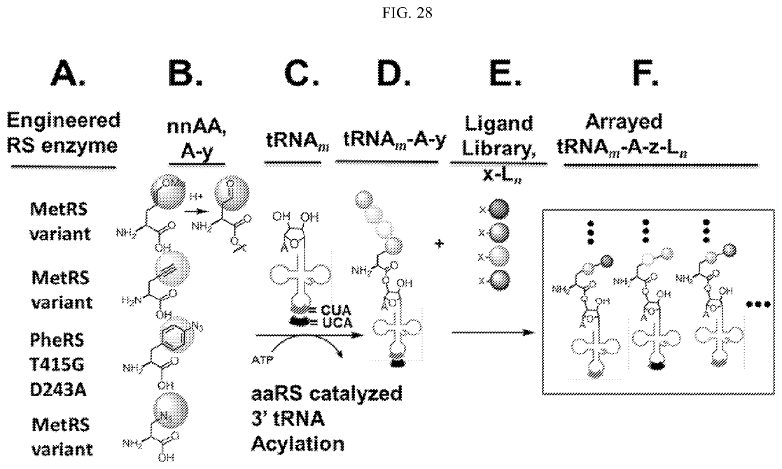

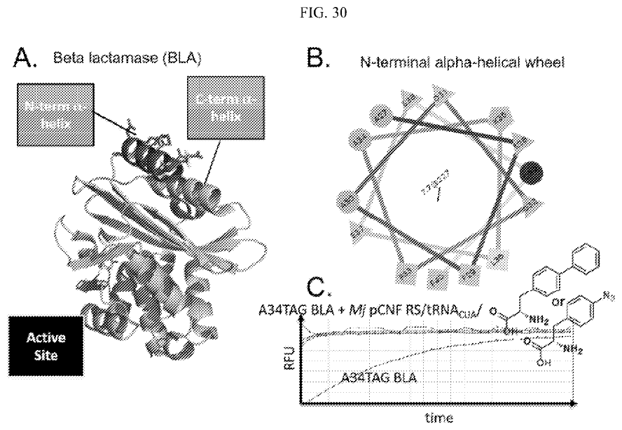

For high-throughput cell-free expression of an polypeptide library coding for 3 positions (i, i+4, and/or i+7) of surface exposed residues in the N-terminus of β-lactamase (BLA; PDB ID: 1BTL) with 20 ligand adduct moieties in Table I, encoded with two stop codons (TAG and TGA) (FIG. 28C), a randomized DNA library of 240 linear templates spatially arrayed (2 suppressor acyl-tRNAs each with 20 ligand adduct specifying barcodes=(20+20) barcodes*(2 codons)3 sites=240 templates) are amplified by PCR from ca. 500 bp DNA synthesized fragments (IDT DNA, Coreaville, Iowa) containing the 5′ untranslated region (UTR), T7 promoter sequence, ribosomal binding sequence (RBS), and a portion of the N-terminal BLA gene sequence. The portion of the BLA gene coding for the C-terminus and the T7 terminator sequence are amplified as well. A GC-rich sequence ChiT2 is used for subsequent overlapping PCR amplification of the full-length template in 96-well format using a single primer, based on the procedure of Woodrow et al. Woodrow, K. A., et al., J Proteome Res (2006) 5:3288-3300. The resulting single band PCR fragments are purified using PureLink™ 96 PCR Purification Kit (Invitrogen), and DNA yields quantitated using PicoGreen. Based on the titration of linear DNA, an average of >30 nM linear template DNA with≈30 μg/mL GamS, is used for each cell-free transcription/translation reaction lacking active RF1, in 96-well plates. Aminoacyl-tRNA_{UCA}^{Met} charged with propargyl glycine ligand adduct moieties prepared by the methods described in Example 9 (Table I) and/or aminoacyl-tRNA_{CUA}^{Tyr} charged with p-acetylPhe ligand adduct moieties prepared by the methods described in Example 10 (Table II) are added to the cell-free reactions in defined positions in 96-well plates, cf. (FIG. 28F & FIG. 30). Cell-free protein synthesis reactions are run for ˜10 min to 1 hr, followed by purification, using Phytip-based affinity chromatography (Phynexus, San Jose, Calif.) or m-Amino phenylboronic acid agarose beads (Sigma-Aldrich, St Louis, Mo.). The translated libraries in ribosome-display format may be pooled, prior to purification.

TABLE 1
Chemical structures of a library of non-canonical amino acid ligand adducts along
with their computed physico-chemical properties (Log P) and van der Waals Volume, in Å³

| | | | van der |
|---|---|---|---|
| | | Log | Waals |
| No. | Structure | P | Vol, Å³ |
| 1 | 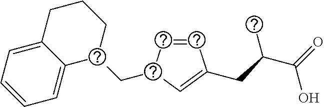 [SMILES: C[C@H](CC1=CC(CC2CCCc3ccccc32)C=C1)C(=O)O] | 1.75 | 270.73 |
| 2 | 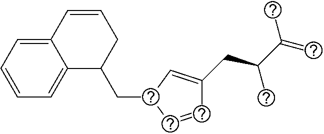 [SMILES: C=C(C)[C@@H](C)CC1=CC(CC2CC=Cc3ccccc32)C=C1] | −2.20 | 262.2 |
| 3 | 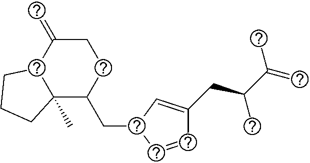 [SMILES: C=C1CCC(CC2C=CC(C[C@H](C)C(=C)C)=C2)[C@@]2(C)CCCC12] | −2.05 | 271 |
| 4 | 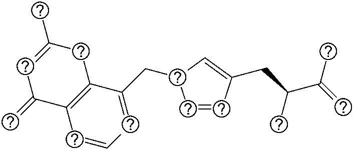 [SMILES: C=C1C=C(C)Cc2c(CC3C=CC(C[C@H](C)C(=C)C)=C3)cccc21] | −1.41 | 263.19 |
| 5 | 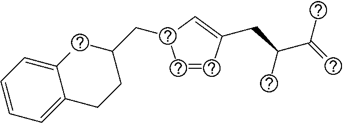 [SMILES: C=C(C)[C@@H](C)CC1=CC(CC2CCc3ccccc3C2)C=C1] | 0.76 | 269.54 |
| 6 | 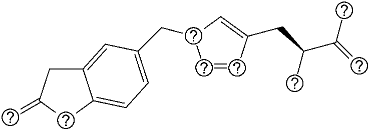 [SMILES: C=C1Cc2ccc(CC3C=CC(C[C@H](C)C(=C)C)=C3)cc2C1] | −0.23 | 253.3 |
| 7 | 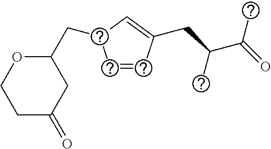 [SMILES: CC(=O)[C@@H](C)CC1=CC(CC2CC(=O)CCO2)C=C1] | −1.06 | 232 |
| 8 | 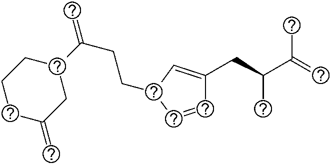 [SMILES: C=C1CCCC(C(=C)CCC2C=CC(C[C@H](C)C(=C)C)=C2)C1] | −3.03 | 266.74 |
| 9 | 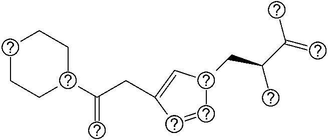 [SMILES: C=C(CC1=CC(C[C@H](C)C(=C)C)C=C1)C1CCCCC1] | −1.90 | 255.98 |
| 10 | 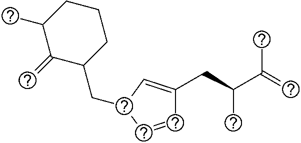 [SMILES: C=C1C(C)CCCC1CC1C=CC(C[C@H](C)C(=C)C)=C1] | −1.30 | 253.15 |
| 11 | 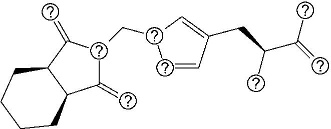 [SMILES: C=C1C(CC2C=CC(C[C@H](C)C(=C)C)=C2)C(=C)[C@@H]2CCCC[C@H]12] | −1.30 | 253.17 |
| 12 | 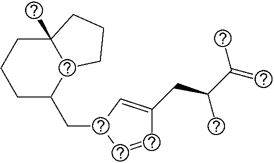 [SMILES: C=C(C)[C@@H](C)CC1=CC(CC2CCC[C@]3(C)CCCC23)C=C1] | 0.04 | 274.18 |
| 13 | 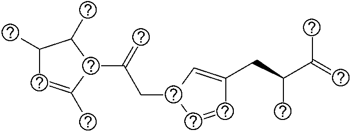 [SMILES: C=C(CC1C=CC(C[C@H](C)C(=C)C)=C1)C1C(C)=CC(C)C1C] | −1.14 | 274.44 |
| 14 | 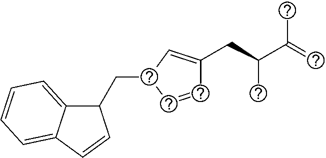 [SMILES: C=C(C)[C@@H](C)CC1=CC(CC2C=Cc3ccccc32)C=C1] | 0.04 | 274.18 |
| 15 | 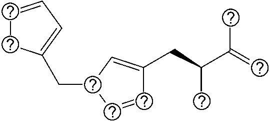 [SMILES: C=C(C)[C@@H](C)CC1=CC(CC2=CC=CC2)C=C1] | −1.17 | 233.3 |
| 16 | 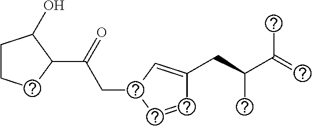 [SMILES: C=C(C)[C@@H](C)CC1=CC(CC(=O)C2CCCC2O)C=C1] | −2.14 | 244.28 |
| 17 | 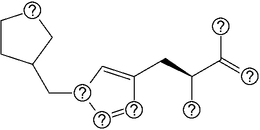 [SMILES: C=C(C)[C@@H](C)CC1=CC(CC2CCCC2)C=C1] | −1.44 | 216.7 |
| 18 | 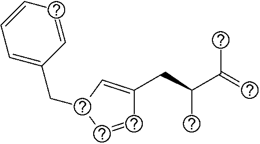 [SMILES: C=C(C)[C@@H](C)CC1=CC(Cc2ccccc2)C=C1] | −0.66 | 214.17 |
| 19 | 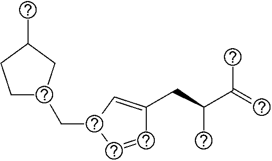 [SMILES: C=C(C)[C@@H](C)CC1=CC(CC2CCC(C)C2)C=C1] | −1.08 | 195.26 |
| 20 | 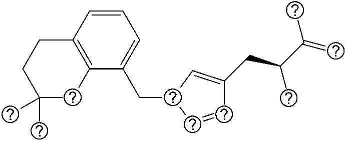 [SMILES: C=C(C)[C@@H](C)CC1=CC(Cc2cccc3c2CC(C)(C)CC3)C=C1] | 1.26 | 301.53 |

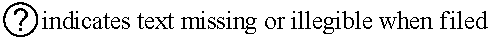

### Example 18

### Decoding the Chemical Structures of Ligand Adducts Using Recovered mRNA

This example describes a representative method for determining chemical structures for ligand adduct polypeptide sequences, using mRNA sequences linked with the translated polypeptide for ribosome display. DNA sequences for each member of the library of non-canonical amino acid ligand adducts in Table I, are designed with a unique sequence associated with a given ligand adduct structure in the 3′ spacer region of a derivative of pRDV ribosome display vector (Dreier, B. and Pluckthun, A., Methods Mol Biol (2012) 805:261-286) using synonymous codons. This ensures that each library member will code for the same spacer amino acid sequence, but with a unique RNA/DNA barcode sequence for each ligand adducts structure. The individually translated beta-lactamase polypeptides, displayed on ribosomes are pooled after initial transcription/translation reactions, then selected for binding to m-phenylboronic acid beads, to select for correctly folded variants.

The beads are washed, then recovered mRNA is reverse transcribed to the corresponding DNA sequences, which are then amplified using specific primers complementary to the constant sequence regions of the vector insert, and harboring 454 Titanium adaptor sequences. The amplified libraries are sequenced using the Roche/454 Genome Sequencer FLX according to the 454 Titanium sequencing protocol. A deconvolution procedure is applied (consisting of comparison of sequence reads of the libraries before and after selection and by the choice of a restricted set of sub-library components for the next round), restricting the number of candidate ligands capable of giving specific binders after cell-free protein synthesis.

### Example 19

### Ribosome Display Selections from Ligand Adduct Libraries

Genetic Library Construction.

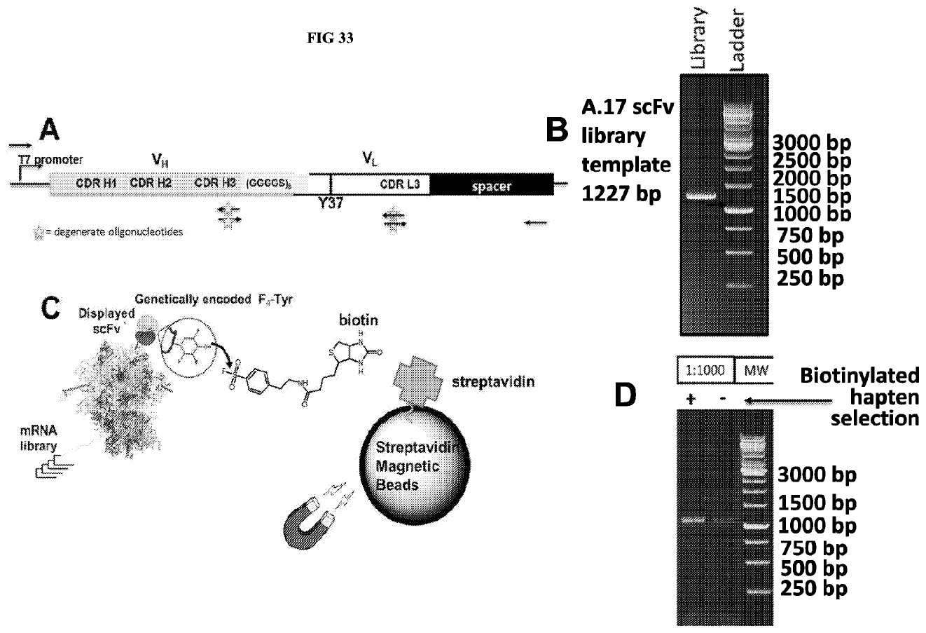

The DNA constructs and their assembly by PCR have been described in detail previously for ribosome display Methods in Molecular Biology (2012) 805: and T7-based cell-free protein synthesis vectors Zawada, J. F., et al., Biotechnol Bioeng (2011) 108:1570-1578. The V_{H} and V_{L} genes from the previously described A.17 Fab antibody Smirnov, I., et al., Proc Natl Acad Sci USA (2011) 108:15954-15959 with a (GGGS)₅ linker were codon optimized, gene synthesized (DNA 2.0, Inc.), and then cloned into these vectors. A TAG DNA sequence mutation encoding FₙY nnAAs at position V_{L}-Y37 (FIG. 33A) was introduced by QuickChange Mutagenesis using Phusion DNA polymerase (Thermo Fisher). Focused CDR libraries were designed based on the x-ray crystal structure of the starting A.17 Fab bound to a phosphonate inhibitor (PDB ID 2XCZ). Several residues in the heavy chain CDR3 and light chain CDR3 within several angstroms of the bound phosphonate were randomized with a diversity designed to mimic natural human antibody diversity Lee, C. V., et al., J Mol Biol (2004) 340:1073-1093, and then assembled by overlap PCR to yield a 1227 bp fragment (FIG. 33B).

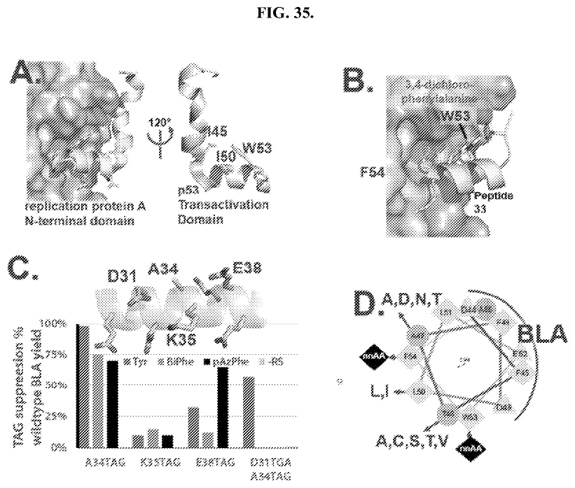

Similarly, a focused library of ligand adducts (FIG. 35D) was designed based on the known interactions of N-terminal domain of RPA bound to an alpha-helical sequence p53 transactivation domain (residues 47-57). (FIG. 35A) and a known alpha-helical peptide (FIG. 35B) interacting with a target protein, the N-terminal domain of RPA.

Hapten Selections.

Ribosome display DNA templates were transcribed and translated using the PURExpress ARF123 kit (New England Biolabs) with the addition of 1 mM 2,3-difluoro tyrosine, 10 μM E11 Mj TyrRS variant, see FIG. 9, and 10 ug/mL pGB014 plasmid transcribed previously. A biotinylated Phenyl fluorosulfonate hapten (FIG. 33C) was synthesized (Tandem Sciences, Inc) and ribosome display selections were performed in the presence or absence of hapten (FIG. 33D), showing enrichment by covalent selection. Using modifications of a previously described covalent selection strategy Smirnov, I., et al., Proc Natl Acad Sci USA (2011) 108:15954-15959; Buchanan, A., et al., Protein Eng Des Sel (2012) 25:631-638 the V_{L}-FₙY37 ribosome display libraries were incubated with biotinylated hapten for 60 min on ice. Library members that bound to biotinylated hapten were recovered on streptavidin beads (Thermo Fisher). Following washing to remove non-bound library members, bound library clones were released from the streptavidin beads using hydroxylamine. Hydroxylamine serves as a mimic of the hydrolytic step to release the covalent adduct. Elution stringency was modulated by incubation time, [NH₂OH], and temperature.

Monitoring and Decoding Library Output.

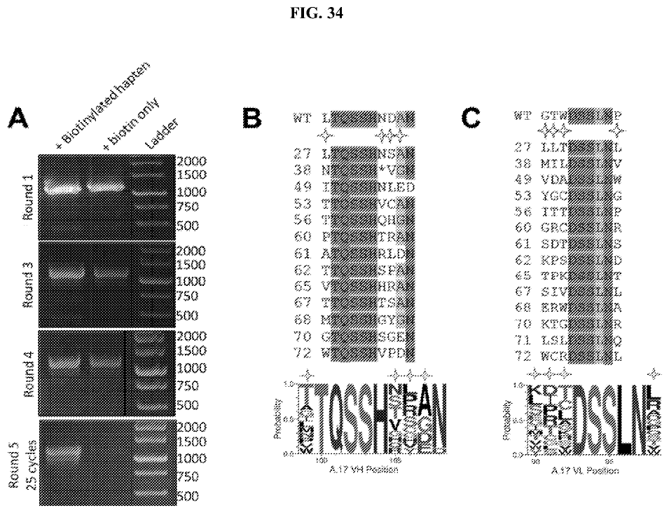

After each round of selection pressure, mRNA output was amplified by RT-PCR using standard protocols (FIG. 34A). The RT-PCR output from the fifth round of selection was subcloned into an appropriate expression vector, transformed into E. coli, and DNA sequences of individual colonies were obtained by Sanger sequencing (Genewiz, LLC) (FIG. 34B).

All references cited herein are incorporated by reference as if each had been individually incorporated by reference in its entirety. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

### Example 20

### Determination of Enzyme Activity of Beta-Lactamase Ligand-Adduct Variants

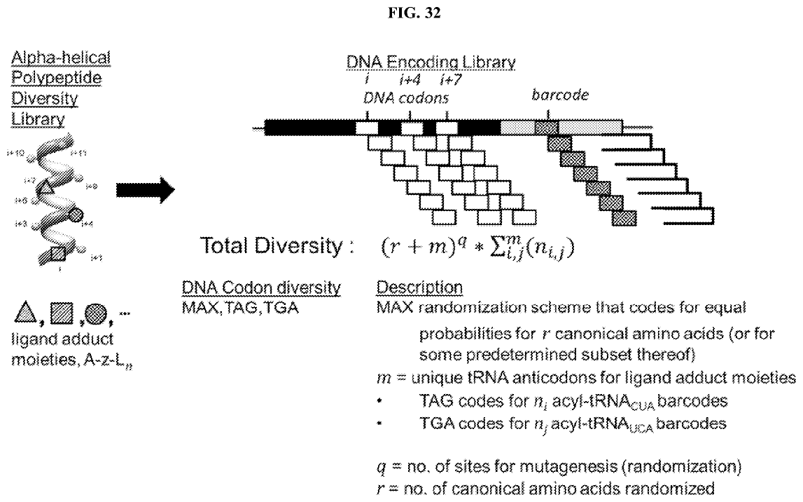

This example illustrates how the N-terminal alpha-helix of Bacillus lichenformis beta-lactamase (BLA; pdb accession 1BLM; FIG. 30A) was used as a scaffold for the display of ligand adduct moieties. The N-terminus of the wild-type BLA protein was extended by a single alpha-helical turn (residues ²⁷AEFA, FIG. 30B) and fused to a streptavidin binding peptide sequence (SBP-Tag2; Barrette-Ng, I. H., et al., Acta Crystallogr D Biol Crystallogr (2013) 69:879-887) using standard PCR assembly and restriction digest cloning protocols. The N-terminus alpha-helix extension provides additional residue mutation possibilities along a single face of the alpha-helix (i.e. positions i, i+4, i+7, etc.; see FIG. 32). Variants, such as A34TAG, were synthesized in 40 μl cell-free transcription/translation reactions overnight at 30° C. with gentle shaking (see Example 15) in the presence or absence of Mj pCNF RS/tRNA_{CUA}/ncAAs (FIG. 35C). The entire reaction volume was then added to a previously blocked and washed streptavidin coated high binding capacity 96-well plate (Thermo Fisher) and incubated for 30 min at room temperature. The wells were washed thrice with ELISA wash buffer (25 mM Tris-HCl, pH 7.5, 150 mM NaCl, 0.1% BSA, 0.05% Tween-20). To each well, PBS supplemented with 10 μM Fluorocillin-Green substrate (Life Technologies) was added and the plate was read using a SpectraMax M2 plate reader (Molecular Devices, Sunnyvale, Calif.) at Ex/Em 495 nm/525 nm for 2 h (FIG. 30C). Enzyme-catalyzed fluorescence activity indicates folded and functional BLA ligand adduct variants were formed. In the absence of the orthoganol translation system; a small amount of read-through incorporation of canonical amino acids is observed.

1. An aminoacyl-tRNA analogue capable of ribosome-directed translation having a structure: 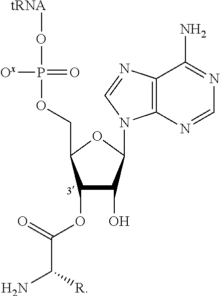
wherein R₁ is a ligand adduct moiety linked to a non-natural amino acid side chain.

2. An acyl-tRNA analogue capable of ribosome-directed translation having a structure:
tRNA-A-z-L
wherein:
tRNA has a 3′ terminus to which at least one hydroxyacyl or aminoacyl group may be transferred;
A is an aminoacyl or α-hydroxyacyl group selected from the group consisting of canonical amino acids, α-hydroxyl acids, non-canonical amino acids and α-hydroxyl acids, each with an orthogonally reactive moiety y;
L is a ligand with a reactive moiety x and
z is a covalent linker formed by a reaction of tRNA-A-y with x-L.

3. The acyl-tRNA analogue according to claim 2, where the aminoacyl group comprises at least one non-canonical amino acid with an orthogonally reactive moiety y.

4. The acyl-tRNA analogue according to claim 2, wherein the 3′ cytosine C75 is not 2′-deoxycytosine.

5. A method of reacting a starting aminoacyl-tRNA compound represented by a structural formula:
tRNA-A-y
wherein A is a non-canonical amino acid with an orthogonally reactive moiety y,
with a ligand, x-L, containing a reactive moiety x, under conditions suitable for a reaction, the method comprising forming a covalent linker of tRNA-A-y with x-L, the covalent linker forming a covalently linked product aminoacyl-tRNA analogue tRNA-A-z-L in greater than 50% yield, relative to the starting tRNA-A-y and wherein the product aminoacyl-tRNA analogue is capable of ribosome-directed translation.

6. The method according to claim 5, wherein the conditions suitable for a reaction comprise an acidic pH.

7. The method according to claim 6, wherein the pH is approximately between about 1 and about 5.

8. The method according to claim 7, wherein then pH is about 5.

9. The method according to claim 5, wherein the starting aminoacyl-tRNA is produced substantially pure in vitro by enzymatic aminoacylation with an engineered aaRS, a tRNA or a non-canonical amino acid.

10. The method according to claim 5, wherein the aminoacyl-tRNA is produced by transcription of a tRNA-ribozyme encoded DNA template with treatment of polynucleotide kinase.

11. The method according to claim 5, wherein the starting aminoacyl-tRNA is produced substantially pure by a T4 RNA ligase coupling of tRNA(-CA) with a non-canonical aminoacyl-pdCpA.

12. The method according to claim 5, wherein x and y are independently selected from the group consisting of (a) an azide as either x or y and an alkyne as the other; (b) an alkene as either x or y and a thiol or an amine as the other (c) a vinyl sulfone as either x or y and a thiol or an amine as the other; (d) an α-halo-carbonyl as either x or y and a thiol or an amine as the other; (e) a disulfide as either x or y and a thiol as the other; (f) a carbonyl as either x or y and an alpha-effect amine as the other; (g) an activated carboxyl ester as either x or y and a primary or aryl amine as the other; (h) a 1,4-dicarbonyl as either x or y and a primary amine as the other; (i) an aryl halide as either x or y and an alkyl or aryl boronate ester; and (j) an aryl halide as either x or y and an alkyne as the other.

13. The method according to claim 5, wherein x and y are masked or protected by reactive functional groups.

14. The method according to claim 5, wherein the method is employed in in vitro transcription and translation systems.

15. The method according to claim 14, wherein a polypeptide containing a site-specific ligand adduct moiety linked to a non-natural amino acid side chain is synthesized by in vitro translation from an mRNA template containing a selector codon at specific sites.

16. The method according to claim 15, wherein the mRNA is encoded by a DNA template containing a selector codon at specific sites.

17. A library formed by reacting
a) an aminoacyl-transfer RNA having formula tRNAₘ-A-y, with a preselected anti-codon, m, a preselected non-canonical amino acid comprising an acceptor moiety, A, with a reactive functionality y, with
b) a plurality of ligand moieties (x-L₁, x-L₂, . . . x-Lₙ), each ligand comprising a donor reactive functionality x and one of a plurality of ligand moieties (L₁, L₂, . . . Lₙ) the reaction occurring under conditions sufficient to form a plurality of n transfer RNA ligands (tRNA₁-A-z-L₁, tRNA₂-A-z-L₁, . . . tRNAₘ-A-z-Lₙ), wherein z is a linker formed by reaction of x and y.

18. The library according to claim 17, wherein the reaction has an acidic pH.

19. The library according to claim 18, wherein the pH is between about 1 and about 5.

20. The library according to claim 19, wherein then pH is about 5.

21. The library according to claim 17, wherein the plurality of ligand moieties are unbiased, functionally-biased, target-biased, or focused.

22. The library according to claim 17, wherein the library is spatially addressed or pooled.

23. A method of screening for a compound that binds to a target, the method comprising:
a) providing a library comprising a plurality of predefined tRNAs that are aminoacylated with a plurality of predefined non-canonical amino acid ligand adducts, wherein the predefined aminoacylated-tRNA non-canonical amino acid ligand adducts are contained in one of a preselected spatially addressed array of vessels;
b) adding a DNA or mRNA template directing the translation of one or more polypeptides site-specifically modified with the predefined non-canonical amino acid ligand adducts;
c) adding a translation system that synthesizes one or more polypeptides site-specifically modified with the predefined non-canonical amino acid ligand adducts;
d) contacting a target with the polypeptides under conditions that permit binding between the target and the polypeptides; and
e) isolating and/or identifying polypeptide products of the translation system that bind to the target.

24. The method according to claim 23, wherein a step c′ is added whereby the translated polypeptides are linked to their encoding mRNA sequences and pooled, followed by steps d and e.

25. The method according to claim 23, wherein the method further comprises adding a predetermined barcode to the DNA or mRNA template, wherein the predetermined barcode is uniquely associated with a non-canonical amino acid and the unique association may be used to identify polypeptides products of the translation system in step (e).

26. The method according to claim 23, wherein identifying the polypeptides products that bind to the target further comprises determining at least the sequence of canonical and non-canonical amino acid ligand adducts comprising the polypeptide products.

27. The method according to claim 26, wherein identifying at least the sequence of canonical and non-canonical amino acid ligand adducts comprising the polypeptide products comprises determining the sequence of the associated mRNA and/or RNA barcodes.

28. The method according to claim 23 wherein the binding is catalytic.

29. The method according to claim 23, wherein the target is another polypeptide.

30. The method according to claim 29, wherein the method is carried out using the library of claim 17.

31. The method according to claim 23, wherein hits obtained from screening are screened against another biological molecule of interest to ascertain differences in an affinity parameter of the hits for the target of interest as against the another biological molecule.

32. The method according to claim 31, wherein the hits are closely related to another biological molecule.

33. The method according to claim 2, wherein x and y are independently selected from the group consisting of (a) an azide as either x or y and an alkyne as the other; (b) an alkene as either x or y and a thiol or an amine as the other (c) a vinyl sulfone as either x or y and a thiol or an amine as the other; (d) an α-halo-carbonyl as either x or y and a thiol or an amine as the other; (e) a disulfide as either x or y and a thiol as the other; (f) a carbonyl as either x or y and an alpha-effect amine as the other; (g) an activated carboxyl ester as either x or y and a primary or aryl amine as the other; (h) a 1,4-dicarbonyl as either x or y and a primary amine as the other; (i) an aryl halide as either x or y and an alkyl or aryl boronate ester; and (j) an aryl halide as either x or y and an alkyne as the other.


SEQUENCE LISTING (1 sequence)

SEQ ID NO: 1 - RNA, 76 nt, Artificial Sequence
  Synthetic
gcggauuuagcucaguugggagagcgccauacucuaaauguggagguccuguguucgauucacagaauucgcacca